(19) United States
(12) Patent Application Publication
(10) Pub. No.: US 2006/0247260 A1
(43) Pub. Date: Nov. 2, 2006

(54) Bis (hetero) aryl carboxamide derivatives for use as PG12 antagonists

(75) Inventors: Toshiki Murata, Nara-ken (JP); Takuya Shintani, Kyoto-fu (JP); Masaomi Umeda, Shiga-ken (JP); Takashi Iino, Kyoto-fu (JP); Toshiya Moriwaki, Nara-ken (JP)

Correspondence Address: JEFFREY M. GREENMAN, BAYER PHARMACEUTICALS CORPORATION, 400 MORGAN LANE, WEST HAVEN, CT, 06516, US

(73) Assignee: BAYER HEALTHCARE AG, LEVERKUSEN (DE)

(21) Appl. No.: 10/544,033

(22) PCT Filed: Jan. 28, 2004
(86) PCT No.: PCT/EP04/00711

§ 371 Date: Jun. 26, 2006

(30) Foreign Application Priority Data
Feb. 10, 2003 (EP) 03002607.4

(51) Int. Cl. A61K 31/513; A61K 31/4412; A61K 31/42; A61K 31/4152; A61K 31/195

(52) U.S. Cl. 514/269; 514/378; 514/355; 514/563; 546/315; 562/450; 544/314; 548/243; 514/406; 548/374.1

(57) ABSTRACT

This invention relates to aryl or heteroaryl amido alkane derivatives of formula (I) 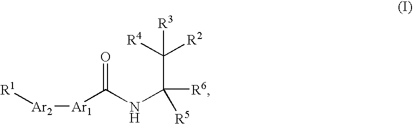
in which Ar₁ and Ar₂ independently represent phenyl or a 5 or 6-membered heteroaromatic ring, R⁶ represents carboxyl or tetrazolyl, and the remaining variables are as defined in the text and claims, which are useful as an active ingredient of pharmaceutical preparations. The aryl or heteroaryl amido alkanes of the present invention have PGI2 antagonistic activity, and can be used for the prophylaxis and treatment of diseases associated with PGI2 activity. Such diseases include urological diseases or disorder as follows: bladder outlet obstruction, overactive bladder, urinary incontinence, detrusor hyper-reflexia, detrusor instability, reduced bladder capacity, frequency of micturition, urge incontinence, stress incontinence, bladder hyperreactivity, benighn prostatic hypertrophy (BPH), prostatitis, urinary frequency, nocturia, urinary urgency, pelvic hypersensitivity, urethritis, pelvic pain syndrome, prostatodynia, cystitis, or idiophatic bladder hypersensitivity. The compounds of the present invention are also useful for treatment of pain including, but not limited to inflammatory pain, neuropathic pain, acute pain, chronic pain, dental pain, premenstrual pain, visceral pain, headaches, and the like; hypotension;hemophilia and hemorrhage; and inflammation, since the diseases also relate to PGI2.

### DETAILED DESCRIPTION OF INVENTION

1. Technical Field

The present invention relates to an aryl or heteroaryl amido alkane derivatives which are useful as an active ingredient of pharmaceutical preparations. The aryl or heteroaryl amido alkane derivatives of the present invention have PGI2 [prostaglandin I2, prostacyclin] antagonistic activity, and can be used for the prophylaxis and treatment of diseases associated with PGI2 activity.

More specifically the aryl or heteroaryl amido alkane derivatives of the present invention are useful for treatment and prophylaxis of urological diseases or disorders.

The compounds of the present invention are also useful for treatment of pain; hypotension; hemophilia and hemorrhage; inflammation; respiratory states from allergies or asthma, since the diseases also relate to PGI2.

2. Background Art

Prostaglandins (or prostanoids, PGs) are a group of bioactive lipid mediators generated from membrane phospholipids. They are formed from 20-carbon essential fatty acids containing 3, 4, or 5 double bonds, and carry a cyclopentane ring. They are divided into 6 main classes (D, E, F, G, H or I) by the cyclopentane ring structure. The main classes are further subdivided by subscripts 1, 2, or 3, reflecting their fatty acid precursors. PGI2 is a member of prostanoids, and it has a double ring structure and is derived from arachidonic acid. The receptor for PGI2 is a seven transmembrane G-protein coupled receptor, called IP. IP couples at least to Gs-type G-protein, and activates adenylate cyclase and phospholipase C. The expression of IP is demonstrated in aorta, coronary/pulmonary/cerebral arteries, platelets, lung, and dorsal root ganglions in addition to several other tissues.

One of the well-known actions of PGI2 is for blood vessels to cause vasodilation and hypotension. Especially in septic shock, PGI2 is produced and participate in the induction of systemic hypotension (G. D. Bottoms et al, Am J Vet Res 1982, 43(6), 999-1002). Therefore, IP receptor antagonists may prevent hypotension associated with septic shock.

Another well-known action of PGI2 is for platelets to suppress aggregation. In the IP receptor knock out mice, FeCl₃-induced thrombosis formation was enhanced compared to that in wild type mice (T. Murata et al, Nature 1997, 388, 678-682.), confirming the involvement of IP receptor in the platelet inhibition. Therefore, IP receptor antagonists may enhance the platelet activation and suppress excessive bleeding such as, but not limited to, hemophilia and hemorrhage.

PGI2 also participate in the inflammation. In the inflamed tissue, various inflammatory mediators, including prostaglandins, are produced. PGI2 is also generated and induces vasodilation to increase blood flow. This enhances vascular permeability, edema formation and leukocyte inflammation in the inflamed region (T. Murata et al, Nature 1997, 388, 678-682.). Therefore, PGI2 receptor antagonists may be efficacious for the treatment of inflammation.

PGI2 may be involved in the pathogenesis of respiratory allergy or asthma. It is spontaneously generated and the major prostaglandin in human lung, and the appropriate antigen challenge increases PGI2 production (E. S. Schulman et al, J Appl Physiol 1982, 53(3), 589-595.). Therefore, IP antagonists may have a utility for the treatment of those respiratory diseases.

In addition, an important role of IP receptor in the induction of hyperalgesia has been clearly shown by IP receptor knockout mice (T. Murata et al., Nature 1997, 388, 678-682.). Injection of acetic acid into the peritoneal cavity induced production of PGI2. This PGI2 is considered to bind to IP receptor on sensory neurons. As IP receptor couples to the activation of both adenylate cyclase and phospholipase C, cAMP-dependent protein kinase (PKA) and protein kinase C (PKC) are activated. PKA and PKC are known to modulate ion channels on sensory neurons such as VR1, P2X3, and TTX-R. As a result, PGI2 sensitizes sensory neurons to enhance the release of neurotransmitters. Hence, acetic acid injection induces nociceptive response (writhing) in mice. This acetic acid-induced writhing was greatly reduced in PGI2 receptor-null mice as the same level as indomethacin-treated wild type mice. Several other in vivo hyperalgesia studies in rodents and in vitro studies further support that PGI2 plays a major role in the induction of hyperalgesia and that PGI2 acts as important modulator of sensory neurons (K. Bley et al, Trends in Pharmacological Sciences 1998, 19(4), 141-147.). Therefore, PGI2 receptor antagonists may be useful for the treatment of pain.

Sensory neurons play very important roles not only in the pain sensation but also in the sensation of bladder distension. In normal subjects, A-delta sensory fibers are considered to play a major role to sense the bladder distention. However, in disease conditions of overactive bladder by, but not limited to, spinal cord injury, cystitis, Parkinson's disease, multiple sclerosis, previous cerebrovascular accident, and bladder outlet obstruction (BOO) caused by benign prostate hyperplasia (BPH), the sensitivity of C-fiber sensory neurons is upregulated and they contribute to the induction of the lower urinary tract symptoms. Treatment of overactive bladder patients with intravesical injection of capsaicin or its potent analog, resiniferatoxin, both of which desensitize VR1-positive C-fiber afferent neurons innervating the bladder, has been shown to be efficacious in several clinical trials (C. Silva et al, Eur Urol. 2000, 38(4), 444-452.). Therefore, C-fiber sensory neurons play an important role in the pathology of overactive bladder. PGI2 is generated locally in the bladder and it is the major prostaglandin released from the human bladder. In a rabbit BOO model, a stable metabolite of PGI2 was reported to be increased in BOO bladder (J M. Masick et al, Prostaglandins Other Lipid Mediat. 2001, 66(3), 211-219.). Hence, PGI2 from disease bladder sensitizes C-fiber sensory neurons, and as a result, it may induce symptoms of overactive bladder. Therefore, antagonists of PGI2 receptor are expected to be useful in the treatment of overactive bladder and related urinary disorders.

WO 98/44797 discloses integrin antagonists and farnesyl protein transferase inhibitors represented by the general formula:

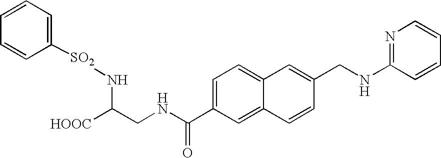

EP-A-220118 discloses pharmaceutical composition intended for the treatment of dermatological, respiratory and ophthalmological conditions represented by the general formula:

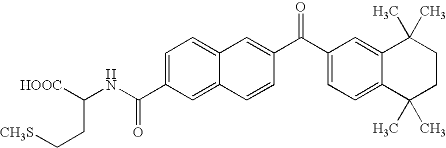

WO 00/43369 discloses pharmaceutical composition intended for the treatment of immune or inflammatory disorders represented by the general formula: 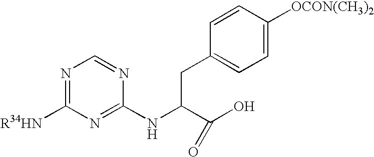
wherein R³⁴ is optionally substituted alkyl, optionally substituted aryl or optionally substituted heteroaryl.

WO9936393 discloses compound and pharmaceutical composition intended for the treatment conditions caused by α₄ mediated cell adhesion represented by the general formula: 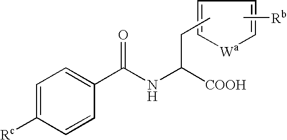
wherein W^{a} is —CH═CH— or a —N═CH—, R^{b} is a substituted or a unsubstituted phenyl or a substituted or a unsubstituted heteroaryl, and R^{c} is a substituted or a unsubstituted aryl or a substituted or a unsubstituted heterocyclic.

The development of a compound which has effective PGI2 antagonistic activity and can be used for the prophylaxis and treatment of diseases associated with PGI2 activity, has been desired.

### SUMMARY OF THE INVENTION

As the result of extensive studies on chemical modification of aryl or heteroaryl amido alkane derivatives, the present inventors have found that the compounds of the structure related to the present invention have unexpectedly excellent PGI2 and/or antagonistic activity. The present invention has been accomplished based on these findings.

This invention is to provide a novel aryl or heteroaryl amido alkane derivative of the formula (I), its tautomeric or stereoisomeric form, or a salt thereof: 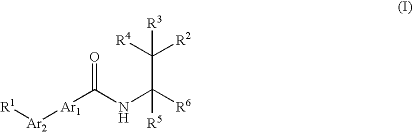
wherein
Ar₁ and Ar₂ independently represent phenyl, or a 5 or 6 membered heteroaromatic ring containing 1 to 4 heteroatoms selected from the group consisting of O, N, and S,
   wherein
said phenyl and 5 or 6 membered heteroaromatic ring are optionally having 1 to 3 substituents selected from the group consisting of halogen and (C₁₋₆)alkyl optionally substituted by hydroxy, or mono-, di- or tri-halogen;
R¹ represents —OR¹¹, —SR¹¹, —SOR¹¹, —SO₂R¹¹, —NR¹²R¹³, —CHR¹⁴R¹⁵, halogen, hydroxy, cyano, nitro, amino, N—(C₁₋₆)alkylamino, N,N-di(C₁₋₆)alkylamino, aryl, heteroaryl,
   (C₁₋₆)alkyl optionally substituted by hydroxy, or mono-, di- or tri-halogen,
   (C₁₋₆)alkoxy optionally substituted by hydroxy, or mono-, di- or tri-halogen,
   aryl substituted (C₁₋₆)alkoxy(C₁₋₆)alkylene, or
   heteroaryl substituted (C₁₋₆)alkoxy(C₁₋₆alkylene,
      wherein
   R¹¹ represents (C₁₋₆)alkyl optionally substituted by a 3 to 10 membered saturated or unsaturated ring having 0 to 3 heteroatoms selected from the group consisting of S, O and N, (C₂₋₆)alkenyl optionally substituted by aryl or heteroaryl, or (C₂₋₆)alkynyl optionally substituted by aryl or heteroaryl,
      wherein
      said 3 to 10 membered saturated or unsaturated ring having 0 to 3 heteroatoms, aryl and heteroaryl are optionally having 1 to 3 substituents selected from the group consisting of halogen, hydroxy, cyano, nitro, amino, N—(C₁₋₆)alkylamino, N,N-di(C₁₋₆) alkylamino, aryl, heteroaryl, (C₁₋₆)alkyl optionally substituted by hydroxy, or mono-, di- or tri-halogen, and (C₁₋₆)alkoxy optionally substituted by mono-, di- or tri-halogen,
   R¹² and R¹³ independently represent hydrogen, (C₁₋₆) alkyl optionally substituted by aryl or heteroaryl, or
   R¹² and R¹³ together form with the nitrogen atom, a 5 to 7 membered saturated heterocyclic ring optionally interrupted by O or NH;
   R¹⁴ and R¹⁵ independently represent hydrogen, aryloxy, heteroaryloxy, (C₁₋₆) alkyl optionally substituted by aryl, heteroaryl, aryloxy, or heteroaryloxy,
      (C₂₋₆)alkenyl optionally substituted by aryl or heteroaryl, or
      (C₂₋₆)alkynyl optionally substituted by aryl or heteroaryl,
         or
   R¹⁴ and R¹⁵ together form, with the CH, a 3-8 membered saturated ring optionally interrupted by NH, or O, or phenyl optionally substituted by hydroxy, halogen or (C₁₋₆) alkyl;
   R² represents hydrogen, hydroxy, halogen, cyano, (C₁₋₆)alkoxy, (C₂₋₆)alkenyl, (C₂₋₆)alkynyl, (C₃₋₇)cycloalkyl, amino, N—(C₁₋₆)alkylamino, N,N-di(C₁₋₆)alkyl-amino, aryl, (C₁₋₆) alkyl optionally substituted by mono-, di- or tri-halogen, or a 5 or 6 membered heteroaromatic ring containing 1 to 4 heteroatoms selected from the group consisting of O, N, and S,
      wherein
      said aryl and 5 or 6 membered heteroaromatic ring are optionally having 1 to 3 substituents selected from the group consisting of halogen, hydroxy, (C₁₋₆)alkyl, (C₁₋₆)alkoxy, amino, N—(C₁₋₆) alkylamino, N,N-di(C₁₋₆)alkylamino, phenyl and a 5 or 6 membered heteroaromatic ring containing 1 to 4 heteroatoms selected from the group consisting of O, N, and S,
         wherein
      said phenyl and 5 or 6 membered heteroaromatic ring are optionally having 1 to 3 substituents selected from the group consisting of halogen, hydroxy, (C₁₋₆)alkyl, (C₁₋₆)alkoxy, amino, N—(C₁₋₆) alkylamino, and N,N-di(C₁₋₆)alkylamino;
   R³ represents hydrogen, hydroxy, halogen, cyano, (C₁₋₆)alkoxy, (C₂₋₆)alkenyl, (C₂₋₆)alkynyl, (C₃₋₇)cycloalkyl, amino, N-C₁₋₆)alkylamino, N,N-di(C₁₋₆)alkylamino, phenyl, (C₁₋₆)alkyl optionally substituted by hydroxy or mono-, di- or tri-halogen, or a 5 or 6 membered heteroaromatic ring containing 1-4 heteroatoms selected from the group of O, N, and S,
      wherein
      said phenyl and 5 or 6 membered heteroaromatic ring are optionally having 1 to 3 substituents selected from the group consisting of halogen, hydroxy, (C₁₋₆)alkyl, (C₁₋₆)alkoxy, amino, N—(C₁₋₆) alkylamino, N,N-di(C₁₋₆) alkylamino, phenyl and a 5 or 6 membered heteroaromatic ring containing 1 to 4 heteroatoms selected from the group consisting of O, N, and S,
         wherein
      said phenyl and 5 or 6 membered heteroaromatic ring are optionally having 1 to 3 substituents selected from the group consisting of halogen, hydroxy, (C₁₋₆)alkyl, (C₁₋₆)alkoxy, amino, N—(C₁₋₆) alkylamino, and N,N-di(C₁₋₆)alkylamino;
   R⁴ represents hydrogen, hydroxy, halogen, cyano, (C₁₋₆)alkoxy, (C₂₋₆)alkenyl, (C₂₋₆)alkynyl, (C₃₋₇)cycloalkyl, amino, N—(C₁₋₆)alkylamino, phenyl, (C₁₋₆)alkyl optionally substituted by hydroxy, mono-, di- or tri-halogen, or a 5 or 6 membered heteroaromatic ring containing 1 to 4 heteroatoms selected from the group consisting of O, N, and S,
      wherein
      said phenyl and 5 or 6 membered heteroaromatic ring are optionally having 1 to 3 substituents selected from the group consisting of halogen, hydroxy, (C₁₋₆)alkyl, (C₁₋₆)alkoxy, amino, N—(C₁₋₆)alkylamino, N,N-di(C₁₋₆)alkylamino, phenyl and a 5 or 6 membered heteroaromatic ring containing 1 to 4 heteroatoms selected from the group consisting of O, N, and S,
         wherein
      said phenyl and 5 or 6 membered heteroaromatic ring are optionally having 1 to 3 substituents selected from the group consisting of halogen, hydroxy, (C₁₋₆)alkyl, (C₁₋₆)alkoxy, amino, N—(C₁₋₆) alkylamino, and N,N-di(C₁₋₆)alkylamino;
   R⁵ represents hydrogen, halogen, cyano, or (C₁₋₆)alkyl optionally substituted by hydroxy or mono-, di- or tri-halogen; and
   R⁶ represents carboxy or tetrazolyl.

The compounds of the present invention surprisingly show excellent PGI2 antagonistic activity. They are, therefore, suitable for the production of medicament or medical composition; which may be useful to treat PGI2 related diseases.

More specifically, since the aryl or heteroaryl amido alkane derivatives of the present invention antagonize PGI2, they are useful for treatment and prophylaxis of urological diseases or disorder.

The compounds of the present invention are also useful for treatment of urological diseases or disorders. Such diseases or disorders include bladder outlet obstruction, overactive bladder, urinary incontinence, detrusor hyper-reflexia, detrusor instability, reduced bladder capacity, frequency of micturition, urge incontinence, stress incontinence, bladder hyperreactivity, benighn prostatic hypertrophy (BPH), prostatitis, urinary frequency, nocturia, urinary urgency, pelvic hypersensitivity, urethritis, pelvic pain syndrome, prostatodynia, cystitis, or idiophatic bladder hypersensitivity.

The compounds of the present invention are also useful for treatment of pain including, but not limited to, inflammatory pain, neuropathic pain, acute pain, chronic pain, dental pain, premenstrual pain, visceral pain, headaches, and the like; hypotension; hemophilia and hemorrhage; inflammation; respiratory states from allergies or asthma, since the diseases also relate to PGI2.

Yet another embodiment of the compounds of formula (I) are those wherein:

Ar₁ and Ar₂ independently represent phenyl, pyridyl, pyrimidinyl, thienyl, oxazolyl, isoxazolyl, pyrrolyl, imidazolyl, or pyrazolyl

In another embodiment, the present invention provides an aryl or heteroaryl amido alkane derivative of the formula (I-i), its tautomeric or stereoisomeric form, or a salt thereof: 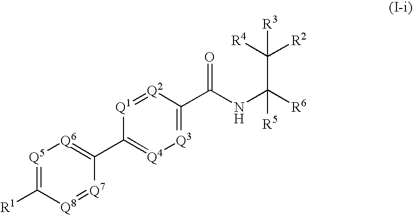
wherein
Q¹, Q², Q³, Q⁴, Q⁵, Q⁶, Q⁷and Q⁸ independently represent CH, CR⁷ or N;
   wherein
R⁷ represents halogen or (C₁₋₆) alkyl optionally substituted by mono-, di- or tri-halogen;
R¹ represents —OR¹¹, —SR¹¹, —SOR¹¹, —SO₂R¹¹, —NR¹²R¹³, —CHR¹⁴R¹⁵, aryl substituted (C₁₋₆)alkoxy(C₁₋₆)alkylene, or heteroaryl substituted (C₁₋₆)alkoxy(C₁₋₆)alkylene,
   wherein
   R¹¹ represents (C₁₋₆) alkyl optionally substituted by a 5 to 7 membered saturated or unsaturated ring having 0 to 3 heteroatoms selected from the group consisting of S, O and N, (C₂₋₆)alkenyl optionally substituted by aryl or heteroaryl, or (C₂₋₆)alkynyl optionally substituted by aryl or heteroaryl,
      wherein
      said 5 to 7 membered saturated or unsaturated ring, aryl and heteroaryl are optionally having 1 to 3 substituents selected from the group consisting of halogen, hydroxy, cyano, nitro, amino, N—(C₁₋₆)alkylamino, N,N-di(C₁₋₆)alkyl-amino, aryl, heteroaryl, (C₁₋₆)alkyl optionally substituted by mono-, di- or tri-halogen, and (C₁₋₆)alkoxy optionally substituted by mono-, di- or tri-halogen;
      R¹² and R¹³ independently represent hydrogen, (C₁₋₆) alkyl optionally substituted by aryl or heteroaryl, (C₂₋₆)alkenyl optionally substituted by aryl or heteroaryl, or (C₂₋₆)alkynyl optionally substituted by aryl or heteroaryl,
         or
      R¹² and R¹³ together form with the nitrogen atom, a 5 to 7 membered saturated heterocyclic ring optionally interrupted by O or NH;
      R¹⁴ and R¹⁵ independently represent hydrogen, aryloxy, heteroaryloxy, (C₁₋₆) alkyl optionally substituted by aryl, heteroaryl, aryloxy, or heteroaryloxy, (C₂₋₆)alkenyl optionally substituted by aryl or heteroaryl, or (C₂₋₆)alkynyl optionally substituted by aryl or heteroaryl,
         or
      R¹⁴ and R¹⁵ together form with the CH, a 5 to 7 membered saturated ring optionally interrupted by NH, O or phenyl optionally substituted by hydroxy, halogen or (C₁₋₆) alkyl;
   R² represents hydrogen, hydroxy, halogen, cyano, (C₁₋₆)alkoxy, (C₂₋₆)alkenyl, (C₂₋₆)alkynyl, (C₃₋₇)cycloalkyl, amino, N—(C₁₋₆)alkylamino, N,N-di(C₁₋₆)alkyl-amino, phenyl, (C₁₋₆)alkyl optionally substituted by hydroxy, or mono-, di- or tri-halogen, or a 5 or 6 membered heteroaromatic ring containing 1 to 4 heteroatoms selected from the group consisting of O, N, and S,
      wherein
      said phenyl and 5 or 6 membered heteroaromatic ring are optionally having 1 to 3 substituents selected from the group consisting of halogen, hydroxy, (C₁₋₆)alkyl, (C₁₋₆)alkoxy, amino, N—(C₁₋₆) alkylamino, N,N-di(C₁₋₆) alkylamino, phenyl and a 5 or 6 membered heteroaromatic ring containing 1 to 4 heteroatoms selected from the group consisting of O, N, and S,
         wherein
      said phenyl and 5 or 6 membered heteroaromatic ring are optionally having 1 to 3 substituents selected from the group consisting of halogen, hydroxy, (C₁₋₆)alkyl, (C₁₋₆)alkoxy, amino, N—(C₁₋₆)alkylamino, and N,N-di(C₁₋₆) alkylamino;
   R³ represents hydrogen, hydroxy, halogen, cyano, (C₁₋₆)alkoxy, (C₂₋₆)alkenyl, (C₂₋₆)alkynyl, (C₃₋₇)cycloalkyl, amino, N—(C₁₋₆)alkylamino, N,N-di(C₁₋₆)alkyl-amino, phenyl, a 5 or 6 membered heteroaromatic ring containing 1 to 4 heteroatoms selected from the group consisting of O, N, and S, or (C₁₋₆) alkyl optionally substituted by mono-, di- or tri-halogen,
      wherein
      said phenyl and 5 or 6 membered heteroaromatic ring are optionally having 1 to 3 substituents selected from the group consisting of halogen, hydroxy, C₁₋₆ alkyl, C₁₋₆ alkoxy, amino, N—(C₁₋₆)alkylamino, N,N-di(C₁₋₆)alkylamino, phenyl and a 5 or 6 membered heteroaromatic ring containing 1 to 4 heteroatoms selected from the group consisting of O, N, and S,
         wherein
      said phenyl and 5 or 6 membered heteroaromatic ring are optionally having 1 to 3 substituents selected from the group consisting of halogen, hydroxy, (C₁₋₆)alkyl, (C₁₋₆)alkoxy, amino, N—(C₁₋₆) alkylamino, and N,N-di(C₁₋₆) alkylamino;
   R⁴ represents hydrogen, hydroxy, halogen, cyano, (C₁₋₆) alkoxy, (C₂₋₆)alkenyl, (C₂₋₆)alkynyl, (C₃₋₇)cycloalkyl, amino, N—(C₁₋₆)alkylamino, N,N-di(C₁₋₆)alkyl-amino, phenyl, (C₁₋₆)alkyl optionally substituted by mono-, di- or tri-halogen, or a 5 or 6 membered heteroaromatic ring containing 1 to 4 heteroatoms selected from the group consisting of O, N, and S,
      wherein
      said phenyl and 5 or 6 membered heteroaromatic ring are optionally having 1 to 3 substituents selected from the group consisting of halogen, hydroxy, C₁₋₆ alkyl, C₁₋₆ alkoxy, amino, N-C₁₋₆)alkylamino, N,N-di(C₁₋₆)alkylamino, phenyl and a 5 or 6 membered heteroaromatic ring containing 1 to 4 heteroatoms selected from the group consisting of O, N, and S,
         wherein
      said phenyl and 5 or 6 membered heteroaromatic ring are optionally having 1 to 3 substituents selected from the group consisting of halogen, hydroxy, (C₁₋₆)alkyl, (C₁₋₆)alkoxy, amino, N—(C₁₋₆) alkylamino, and N,N-di(C₁₋₆)alkylamino;
   R⁵ represents hydrogen, halogen, cyano, or (C₁₋₆)alkyl optionally substituted by mono-, di- or tri-halogen; and
   R⁶ represents carboxy or tetrazolyl.

Yet another embodiment of the compounds of formula (I-i) are those wherein:
Q¹ represents N;
Q², Q³, Q⁴, Q⁵, Q⁶, Q⁷ and Q⁸ independently represent CH or CR⁷;
   wherein
R⁷ represents halogen or (C₁₋₆) alkyl optionally substituted by mono-, di- or tri-halogen;
R¹ represents —OR¹¹,
   wherein
R¹¹ represents (C₁₋₆) alkyl optionally substituted by a 5 to 7 membered saturated or unsaturated ring having 0 to 3 heteroatoms selected from the group consisting of S, O and N,
   wherein
   said 5 to 7 membered saturated or unsaturated ring is optionally having 1 to 3 substituents selected from the group consisting of halogen, hydroxy, cyano, nitro, amino, N—(C₁₋₆)alkylamino, N,N-di(C₁₋₆) alkylamino, aryl, heteroaryl, (C₁₋₆)alkyl optionally substituted by mono-, di- or tri-halogen, and (C₁₋₆)alkoxy optionally substituted by mono-, di- or tri-halogen;
R³, R⁴ and R⁵ represents hydrogen; and
R⁶ represents carboxy.

Another embodiment of the compounds of formula (I-i) is those wherein:
Q² represents N;
Q¹, Q³, Q⁴, Q⁵, Q⁶, Q⁷ and Q⁸ independently represent CH or CR⁷;
   wherein
   R⁷ represents halogen or (C₁₋₆)alkyl optionally substituted by mono-, di- or tri-halogen;
R¹ represents —OR¹¹,
   wherein
   R¹¹ represents (C₁₋₆)alkyl optionally substituted by a 5 to 7 membered saturated or unsaturated ring having 0 to 3 heteroatoms selected from the group consisting of S, O and N,
      wherein
      said 5 to 7 membered saturated or unsaturated ring is optionally having 1 to 3 substituents selected from the group consisting of halogen, hydroxy, cyano, nitro, amino, N—(C₁₋₆) alkylamino, N,N-di(C₁₋₆)alkylamino, aryl, heteroaryl, (C₁₋₆)alkyl optionally substituted by mono-, di- or tri-halogen and (C₁₋₆)alkoxy optionally substituted by mono-, di- or tri-halogen;
R³, R⁴ and R⁵ represents hydrogen; and
R⁶ represents carboxy.

Another embodiment of the compounds of formula (I-i) is those wherein:
Q⁵ and Q⁸ independently represent CH or N;
Q¹, Q², Q³, Q⁴, Q⁶ and Q⁷ independently represent CH or CR⁷;
   wherein
   R⁷ represents halogen or (C₁₋₆)alkyl optionally substituted by mono-, di- or tri-halogen;
R¹ represents —OR¹¹,
   wherein
   R¹¹ represents (C₁₋₆)alkyl optionally substituted by a 5 to 10 membered saturated or unsaturated ring having 0 to 3 heteroatoms selected from the group consisting of S, O and N,
      wherein
      said 5 to 10 membered saturated or unsaturated ring is optionally having 1 to 3 substituents selected from the group consisting of halogen, hydroxy, cyano, nitro, amino, N—(C₁₋₆) alkylamino, N,N-di(C₁₋₆)alkylamino, aryl, heteroaryl, (C₁₋₆)alkyl optionally substituted by mono-, di- or tri-halogen and (C₁₋₆)alkoxy optionally substituted by mono-, di- or tri-halogen;
R³, R⁴ and R⁵ represents hydrogen; and
R⁶ represents carboxy.

Another embodiment of the compounds of formula (I-i) is those wherein:
Q¹ represents N;
Q², Q³, Q⁴, Q⁵, Q⁶, Q⁷ and Q⁸ independently represent CH;
R¹ represents aryl(C₁₋₆)alkoxy, aryl(₁₋₆)alkoxy(C₁₋₆)alkylene or aryloxy(C₁₋₆)alkylene,
   wherein
   said aryl and aryl moiety of said aryloxy are optionally having 1 to 3 substituents selected from the group consisting of halogen, hydroxy, cyano, nitro, amino, N—(C₁₋₆)alkylamino, N,N-di(C₁₋₆)alkylamino, (C₁₋₆)alkyl optionally substituted by mono-, di- or tri-halogen, and (C₁₋₆)alkoxy optionally substituted by mono-, di- or tri-halogen;
R² represents halogen, hydroxy, (C₃₋₇)cycloalkyl, or phenyl optionally having 1 to 3 substituents selected from the group consisting of halogen, hydroxy, amino, N—(C₁₋₆)alkylamino, N,N-di(C₁₋₆)alkylamino, and phenyl optionally substituted by halogen, hydroxy or amino;
R³, R⁴ and R⁵ represents hydrogen; and
R⁶ represents carboxy.

Another embodiment of the compounds of formula (I-i) is those wherein:
Q² represent N;
Q¹, Q³, Q⁴, Q⁵, Q⁶, Q⁷ and Q⁸ independently represent CH;
R¹ represents aryl(C₁₋₆)alkoxy, aryl(C₁₋₆)alkoxy(C₁₋₆)alkylene or aryloxy(C₁₋₆)alkyl;
   wherein
   said aryl and aryl moiety of said aryloxy are optionally having 1 to 3 substituents selected from the group consisting of halogen, hydroxy, cyano, nitro, amino, N—(C₁₋₆)alkylamino, N,N-di(C₁₋₆)alkylamino, (C₁₋₆)alkyl optionally substituted by mono-, di- or tri-halogen, and (C₁₋₆)alkoxy optionally substituted by mono-, di- or tri-halogen;
R² represents halogen, hydroxy, (C₃₋₇)cycloalkyl, or phenyl optionally having 1 to 3 substituents selected from the group consisting of halogen, hydroxy, amino, N—(C₁₋₆)alkylamino, N,N-di(C₁₋₆)alkylamino and phenyl optionally substituted by halogen, hydroxy or amino;
R³, R⁴ and R⁵ represents hydrogen; and
R⁶ represents carboxy.

Another embodiment of the compounds of formula (I-i) is those wherein:
Q⁵ and Q⁸ independently represent CH or N;
Q¹, Q², Q³, Q⁴, Q⁶ and Q⁷ independently represent CH
R¹ represents aryl(C₁₋₆)alkoxy, aryl(C₁₋₆)alkoxy(C₁₋₆)alkylene or aryloxy(C₁₋₆)alkyl;
   wherein
   said aryl and aryl moiety of said aryloxy are optionally having 1 to 3 substituents selected from the group consisting of halogen, hydroxy, cyano, nitro, amino, N—(C₁₋₆)alkylamino, N,N-di(C₁₋₆)alkylamino, (C₁₋₆)alkyl optionally substituted by mono-, di- or tri-halogen, and (C₁₋₆)alkoxy optionally substituted by mono-, di- or tri-halogen;
R² represents halogen, hydroxy, (C₃₋₇)cycloalkyl, or phenyl optionally having 1 to 3 substituents selected from the group consisting of halogen, hydroxy, amino, N—(C₁₋₆)alkylamino, N,N-di(C₁₋₆)alkylamino and phenyl optionally substituted by halogen, hydroxy or amino;
R³, R⁴ and R⁵ represents hydrogen; and
R⁶ represents carboxy.

Another embodiment of the compounds of formula (I-i) is those wherein:
Q¹, Q², Q³, Q⁴, Q⁵, Q⁶, Q⁷ and Q⁸ independently represent CH or CR⁷;
   wherein
   R⁷ represents halogen or (C₁₋₆) alkyl optionally substituted by mono-, di- or tri-halogen;
R¹ represents aryl(C₁₋₆)alkoxy, heteroaryl(C₁₋₆)alkoxy, (C₅₋₇)cycloalkyl(C₁₋₆)alkoxy, aryl(C₁₋₆)alkoxy(C₁₋₆)alkylene or aryloxy(C₁₋₆)alkylene;
   wherein
   said aryl, heteroaryl, (C₅₋₇)cycloalkyl, and aryl moiety of said aryloxy are optionally having 1 to 3 substituents selected from the group consisting of halogen, hydroxy, cyano, nitro, amino, N—(C₁₋₆)alkylamino, N,N-di(C₁₋₆)alkyl-amino, (C₁₋₆)alkyl optionally substituted by mono-, di- or tri-halogen, and (C₁₋₆)alkoxy optionally substituted by mono-, di- or tri-halogen;
R² represents halogen, hydroxy, (C₅₋₇)cycloalkyl, or phenyl optionally having 1 to 3 substituents selected from the group consisting of halogen, hydroxy, amino, N—(C₁₋₆)alkylamino, N,N-di(C₁₋₆)alkylamino and phenyl optionally substituted by halogen, hydroxy or amino;
R³, R⁴ and R⁵ represents hydrogen; and
R⁶ represents carboxy.

Another embodiment of the compounds of formula (I-i) is those wherein:
Q¹ represent N;
Q², Q³, Q⁴, Q⁵, Q⁶, Q⁷ and Q⁸ independently represent CH,
R¹ represents phenyl(C₁₋₆)alkoxy, phenyl(C₁₋₆)alkoxy(C₁₋₆)alkylene or phenoxy(C₁₋₆)alkylene,
   wherein
   said phenyl and phenoxy are optionally having 1 to 3 substituents selected from the group consisting of halogen, hydroxy, cyano, nitro, amino, N—(C₁₋₆)alkylamino, N,N-di(C₁₋₆)alkylamino, (C₁₋₆)alkyl optionally substituted by mono-, di- or tri-halogen and (C₁₋₆)alkoxy optionally substituted by mono-, di- or tri-halogen,;
R² represents halogen, hydroxy, (C₅₋₇)cycloalkyl, or phenyl optionally having 1 to 3 substituents selected from the group consisting of halogen, hydroxy, amino, N—(C₁₋₆)alkylamino, N,N-di(C₁₋₆)alkylamino and phenyl optionally substituted by halogen, hydroxy or amino;
R³, R⁴ and R⁵ represents hydrogen; and
R⁶ represents carboxy.

Another embodiment of the compounds of formula (I-i) is those wherein:
Q² represent N;
Q¹, Q³, Q⁴Q⁵, Q⁶, Q⁷ and Q⁸ independently represent CH;
R¹ represents phenyl(C₁₋₆)alkoxy, phenyl(C₁₋₆)alkoxy(C₁₋₆)alkylene or phenoxy(C₁₋₆)alkylene;,
   wherein
   said phenyl and phenyl moiety of said phenoxy are optionally having 1 to 3 substituents selected from the group consisting of halogen, hydroxy, cyano, nitro, amino, N—(C₁₋₆)alkylamino, N,N-di(C₁₋₆)alkylamino, (C₁₋₆)alkyl optionally substituted by mono-, di- or tri- halogen, and (C₁₋₆)alkoxy optionally substituted by mono-, di- or tri-halogen;
R² represents halogen, hydroxy, (C₅₋₇)cycloalkyl, or phenyl optionally having 1 to 3 substituents selected from the group consisting of halogen, hydroxy, amino, N—(C₁₋₆)alkylamino, N,N-di(C₁₋₆)alkylamino and phenyl optionally substituted by halogen, hydroxy or amino;
R³, R⁴ and R⁵ represents hydrogen; and
R⁶ represents carboxy.

Another embodiment of the compounds of formula (I-i) is those wherein:
Q⁵ and Q⁸ independently represent CH or N;
Q¹, Q², Q³, Q⁴, Q⁶ and Q⁷ independently represent CH;
R¹ represents phenyl(C¹⁻⁶)alkoxy, phenyl(C₁₋₆)alkoxy(C₁₋₆)alkylene or phenoxy(C₁₋₆)alkylene;
   wherein
   said phenyl and phenyl moiety of said phenoxy are optionally having 1 to 3 substituents selected from the group consisting of halogen, hydroxy, cyano, nitro, amino, N—(C₁₋₆)alkylamino, N,N-di(C₁₋₆) alkylamino, (C₁₋₆)alkyl optionally substituted by mono-, di- or tri-halogen, and (C₁₋₆)alkoxy optionally substituted by mono-, di- or tri-halogen;
R² represents halogen, hydroxy, (C₅₋₇)cycloalkyl, or phenyl optionally having 1 to 3 substituents selected from the group consisting of halogen, hydroxy, amino, N—(C₁₋₆)alkylamino, N,N-di(C₁₋₆)alkylamino and phenyl optionally substituted by halogen, hydroxy or amino;
R³, R⁴ and R⁵ represents hydrogen; and
R⁶ represents carboxy.

Another embodiment of the compounds of formula (I-i) is those wherein:
Q¹, Q², Q³, Q⁴, Q⁵, Q⁶, Q⁷ and Q⁸ independently represent CH or CR⁷;
   wherein
   R⁷ represents halogen or (C₁₋₆)alkyl optionally substituted by mono-, di- or tri-halogen;
R¹ represents phenyl(C₁₋₆)alkoxy, phenyl(C₁₋₆)alkoxy(C₁₋₆)alkylene or phenoxy(C₁₋₆)alkylene;
   wherein
   said phenyl and phenyl moiety of said phenoxy are optionally having 1 to 3 substituents selected from the group consisting of halogen, hydroxy, cyano, nitro, amino, N—(C₁₋₆)alkylamino, N,N-di(C₁₋₆)alkylamino, (C₁₋₆)alkyl optionally substituted by mono-, di- or tri-halogen and (C₁₋₆)alkoxy optionally substituted by mono-, di- or tri-halogen;
R² represents halogen, hydroxy, (C₅₋₇)cycloalkyl, or phenyl optionally having 1 to 3 substituents selected from the group consisting of halogen, hydroxy, amino, N—(C₁₋₆)alkylamino, N,N-di(C₁₋₆)alkylamino and phenyl optionally substituted by halogen, hydroxy or amino;
R³, R⁴ and R⁵ represents hydrogen; and
R⁶ represents carboxy.

Another embodiment of the compounds of formula (I-i) is those wherein:
Q¹, Q², Q³, Q⁴, Q⁵, Q⁶, Q⁷ and Q⁸ independently represent CH or CR⁷;
   wherein
   R⁷ represents fluoro, chloro, bromo, or (C₁₋₆) alkyl optionally substituted by mono-, di- or tri-halogen;
R¹ represents (C₁₋₄)alkoxy optionally substituted by phenyl, cyclohexyl, pyrrolyl or piperidino;
   wherein
   said phenyl is optionally having 1 to 3 substituents selected from the group consisting of fluoro, chloro, bromo,. hydroxy, cyano, nitro, amino, methyl, methoxy, trifluoromethyl, and trifluoromethoxy;
R² represents (C₅₋₇)cycloalkyl, or phenyl;
R³, R⁴ and R⁵ represents hydrogen; and
R⁶ represents carboxy.

In another embodiment, the present invention provides an aryl or heteroaryl amido alkane derivative of the formula (I-ii), its tautomeric or stereoisomeric form, or a salt thereof: 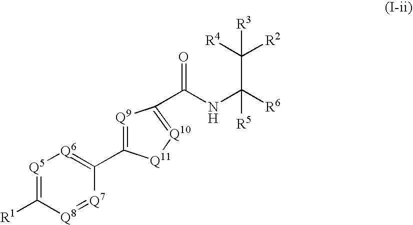
wherein
Q₅, Q⁶, Q⁷, Q⁸, Q⁹ and Q¹⁰ independently represent CH or N;
Q¹¹ represents CH₂, S, NH, or O;
R¹ represents —OR¹¹, —SR¹¹, —SOR¹¹, —SO₂R¹¹, —NR¹²R¹³, —CHR¹⁴R¹⁵,
   aryl substituted (C₁₋₆)alkoxy(C₁₋₆)alkylene, or heteroaryl substituted (C₁₋₆) alkoxy(C₁₋₆) alkylene,
      wherein
   R¹¹ represents (C₁₋₆) alkyl optionally substituted by a 3 to 10 membered saturated or unsaturated ring having 0 to 3 heteroatoms selected from the group consisting of S, O and N, (C₂₋₆)alkenyl optionally substituted by aryl or heteroaryl, or (C₂₋₆)alkynyl optionally substituted by aryl or heteroaryl,
      wherein
      said 3 to 10 membered saturated or unsaturated ring, aryl and heteroaryl are optionally having 1 to 3 substituents selected from the group consisting of halogen, hydroxy, cyano, nitro, amino, N—(C₁₋₆)alkylamino, N,N-di(C₁₋₆) alkylamino, aryl, heteroaryl, (C₁₋₆)alkyl optionally substituted by mono-, di- or tri-halogen, and (C₁₋₆)alkoxy optionally substituted by mono-, di- or tri-halogen,
   R¹² and R¹³ independently represent hydrogen, (C₁₋₆)alkyl optionally substituted by aryl or heteroaryl, (C₂₋₆)alkenyl optionally substituted by aryl or heteroaryl, or (C₂₋₆)alkynyl optionally substituted by aryl or heteroaryl,
      or
   R¹² and R¹³ together form with the nitrogen atom, a 5 to 7 membered saturated heterocyclic ring optionally interrupted by O or NH;
   R¹⁴ and R¹⁵ independently represent hydrogen, aryloxy, heteroaryloxy, (C₁₋₆) alkyl optionally substituted by aryl, heteroaryl, aryloxy, or heteroaryloxy, (C₂₋₆)alkenyl optionally substituted by aryl or heteroaryl, or (C₂₋₆)alkynyl optionally substituted by aryl or heteroaryl,
      wherein
      said aryl and aryl moiety of said aryloxy are optionally having 1 to 3 substituents selected from the group consisting of halogen, hydroxy, cyano, nitro, amino, N—(C₁₋₆)alkylamino, N,N—(C₁₋₆)alkylamino, (C₁₋₆)alkyl optionally substituted by mono-, di- or tri-halogen, and (C₁₋₆)alkoxy optionally substituted by mono-, di- or tri-halogen;
         or
   R¹⁴ and R¹⁵ together form with the CH, a 5 to 7 membered saturated ring. optionally interrupted by NH, or O, or phenyl optionally substituted by hydroxy, halogen or (C₁₋₆) alkyl;
R² represents hydrogen, hydroxy, halogen, cyano, (C₁₋₆)alkoxy, (C₂₋₆)alkenyl, (C₂₋₆)alkynyl, (C₃₋₇)cycloalkyl, amino., N—(C₁₋₆)alkylamino, N,N-di(C₁₋₆)alkylamino; aryl, a 5 or 6 membered heteroaromatic ring containing 1-4 heteroatoms selected from the group of O; N, and S, or (C₁₋₆) alkyl optionally substituted by mono-, di- or tri-halogen,
   wherein
   said aryl and heteroaromatic ring are optionally having 1 to 3 substituents selected from the group consisting of halogen, hydroxy, C₁₋₆ alkyl, C₁₋₆ alkoxy, amino, N—(C₁₋₆)alkylamino, N,N-di(C₁₋₆) alkylamino, phenyl and a 5 or 6 membered heteroaromatic ring containing 1 to 4 heteroatoms selected from the group of O, N, and S,
      wherein
   said phenyl and 5 or 6 membered heteroaromatic ring optionally are having 1 to 3 substituents selected from the group consisting of halogen, hydroxy, C₁₋₆ alkyl, C₁₋₆ alkoxy, amino, N—(C₁₋₆)alkylamino, and N,N-di(C₁₋₆) alkylamino;
R³ represents hydrogen;
R⁴ represents hydrogen;
R⁵ represents hydrogen, hydroxy, cyano, or (C₁₋₆)alkyl optionally substituted by mono-, di- or tri-halogen; and
R⁶ represents carboxy or tetrazolyl.

Another embodiment of the compounds of formula (I-ii) is those wherein:
Q⁵, Q⁶, Q⁷, Q⁸ and Q⁹ represent CH;
Q¹⁰ represents CH or N;
Q¹¹ represents CH₂, NH, or O;
R¹ represents phenyl(C₁₋₆)alkoxy, phenyl(C₁₋₆)alkoxy(C₁₋₆)alkylene or phenoxy(C₁₋₆)alkylene,
   wherein
   said phenyl and phenyl moiety of said phenoxy are optionally having 1 to 3 substituents selected from the group consisting of halogen, hydroxy, cyano, nitro, amino, N—(C₁₋₆)alkylamino, N,N-di(C₁₋₆)alkylamino, (C₁₋₆)alkyl optionally substituted by mono-, di- or tri-halogen, and (C₁₋₆)alkoxy optionally substituted by mono-, di- or tri-halogen;
R² represents halogen, hydroxy, (C₅₋₇)cycloalkyl, or phenyl optionally having 1 to 3 substituents selected from the group consisting of halogen, hydroxy, amino, N—(C₁₋₆)alkylamino, N,N-di(C₁₋₆)alkylamino and phenyl optionally substituted by halogen, hydroxy or amino;
R³ represents hydrogen;
R⁴ represents hydrogen;
R⁵ represents hydrogen; and
R⁶ represents carboxy.

In another embodiment, the present invention provides an aryl or heteroaryl amido alkane derivative of the formula (I-iii), its tautomeric or stereoisomeric form, or a salt thereof: 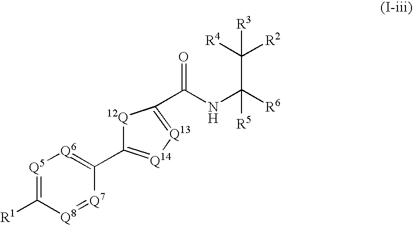
wherein
Q⁵, Q⁶, Q⁷, Q⁸, Q¹³ and Q¹⁴ represent CH;
Q¹² represents CH₂, NH, O, or S;
R¹ represents phenyl(C₁₋₆)alkoxy, phenyl(C₁₋₆)alkoxy(C₁₋₆)alkylene or phenoxy(C₁₋₆)alkylene,
   wherein
   said phenyl and phenyl moiety of said phenoxy are optionally having 1 to 3 substituents selected from the group consisting of halogen, hydroxy, cyano, nitro, amino, N—(C₁₋₆)alkylamino, N,N-di(C₁₋₆)alkylamino, (C₁₋₆)alkyl optionally substituted by mono-, di- or tri-halogen, and (C₁₋₆)alkoxy optionally substituted by mono-, di- or tri-halogen;
R² represents halogen, hydroxy, (C₅₋₇)cycloalkyl, or phenyl optionally having 1 to 3 substituents selected from the group consisting of halogen, hydroxy, ammo, N—(C₁₋₆)alkylamino, N,N-di(C₁₋₆)alkylamino and phenyl optionally substituted by halogen, hydroxy or amino;
R³ represents hydrogen;
R⁴ represents hydrogen;
R⁵ represents hydrogen; and
R⁶ represents carboxy.

More preferably, said aryl or heteroaryl amido alkane derivatives of the formula (I) is selected from the group consisting of:
2-{[5-(4-benzyloxyphenyl)-1H-pyrazole-3-carbonyl]amino}-3-phenylpropionic acid;
2-{[5-(4-benzyloxyphenyl)isoxazole-3-carbonyl]amino}-3-phenylpropionic acid;
2-{[5-(4-benzyloxyphenyl)thiophene-2-carbonyl]amino}-3-phenyl-propionic acid;
2-[(4′-benzyloxybiphenyl-4-carbonyl)amino]-3-phenylpropionic acid;
2-{[6-(4-benzyloxyphenyl)pyridine-3-carbonyl]amino}-3-phenyl-propionic acid;
2-[4-2-benzyloxypyrimidin-5-yl)benzoylamino]-3-phenylpropionic acid;
2-[4-(6-benzyloxypyridin-3-yl)benzoylamino]-3-phenylpropionic acid;
2-[(4′-benzyloxy-3′-fluorobiphenyl-4-carbonyl)amino]-3-phenylpropionic acid;
2-{[4′-(3-methylbenzyloxy)biphenyl-4-carbonyl]amino}-3-phenylpropionic acid;
2-{[4′-(2-Fluoro-benzyloxy)-biphenyl-4-carbonyl]-amino}-3-phenyl-propionic acid;
2-{[4′-(3-Fluoro-benzyloxy) biphenyl-4-carbonyl]-amino}-3-phenyl-propionic acid;
2-{[4′-(4-Fluoro-benzyloxy)-biphenyl-4-carbonyl]-amino}-3-phenyl-propionic acid;
2-{[4′-(3,4-Difluoro-benzyloxy)-biphenyl-4-carbonyl]-amino}-3-phenyl-propionic acid;
2-{[4′-(2,6-Dichloro-benzyloxy)-biphenyl-4-carbonyl]-amino}-3-phenyl-propionic acid;
2-{[4′-(2,3-Dichloro-benzyloxy)-biphenyl-4-carbonyl]-amino}-3-phenyl-propionic acid;
2-{[4′-(3-Methoxy-benzyloxy)-biphenyl-4-carbonyl]-amino}-3-phenyl-propionic acid;
2-{[4′-(2-Chloro-benzyloxy)-biphenyl-4-carbonyl]-amino}-3-phenyl-propionic acid;
2-{[4′-(2,4-Difluoro-benzyloxy)-biphenyl-4-carbonyl]-amino}-3-phenyl-propionic acid;
2-{[4′-(3-Chloro-benzyloxy)-biphenyl-4-carbonyl]-amino}-3-phenyl-propionic acid;
2-{[4′-(3,5-Dimethoxy-benzyloxy)-biphenyl-4-carbonyl]-amino}-3-phenyl-propionic acid;
2-{[4′-(2-Methyl-benzyloxy)-biphenyl-4-carbonyl]-amino}-3-phenyl-propionic acid;
2-{[4′-(3-Nitro-benzyloxy)-biphenyl-4-carbonyl]-amino}-3-phenyl-propionic acid;
2-[(4′-Phenethyloxy-biphenyl-4-carbonyl)-amino]-3-phenyl-propionic acid; and
2-{[4′-(3-Amino-benzyloxy)-biphenyl-4-carbonyl]-amino}-3-phenyl-propionic acid.

Further, the present invention provides a medicament, which includes one of the compounds, described above and optionally pharmaceutically acceptable excipients.

Alkyl per se and “alk” and “alkyl” in alkoxy, alkanoyl, alkylamino, alkylaminocarbonyl, alkylaminosulphonyl, alkylsulphonylamino, alkoxycarbonyl, alkoxyamido and alkanoylamino represent a linear or branched alkyl radical having generally 1 to 6, preferably 1 to 4 and particularly preferably 1 to 3 carbon atoms, representing illustratively and preferably methyl, ethyl, n-propyl, isopropyl, tert-butyl, n-pentyl and n-hexyl.

Alkoxy illustratively and preferably represents methoxy, ethoxy, n-propoxy, isopropoxy, tert-butoxy, n-pentoxy and n-hexoxy.

Alkylamino represents an alkylamino radical having one or two (independently selected) alkyl substituents, illustratively and preferably representing methylamino, ethylamino, n-propylamino, isopropylamino, tert-butylamino, n-pentylamino, n-hexyl-amino, N,N-dimethylamino, N,N-diethylamino, N-ethyl-N-methylamino, N-methyl-N-n-propylamino, N-isopropyl-N-n-propyl-amino, N-t-butyl-N-methylamino, N-ethyl-N-n-pentylamino and N-n-hexyl-N-methylamino.

Aryl per se represents a mono- to tricyclic aromatic carbocyclic radical having generally 6 to 14 carbon atoms, illustratively and preferably representing phenyl, naphthyl and phenanthrenyl.

Heteroaryl represents an aromatic mono- or bicyclic radical having generally 5 to 10 and preferably 5 or 6 ring atoms and up to 5 and preferably up to 4 hetero atoms selected from the group consisting of S, O and N, illustratively and preferably representing thienyl, furyl, pyrrolyl, thiazolyl, oxazolyl, imidazolyl, pyridyl, pyrimidyl, pyridazinyl, indolyl, indazolyl, benzofuranyl, benzothiophenyl, quinolinyl, isoquinolinyl.

5- or 6-membered heteroaromatic rings illustratively and preferably represent tetrazolyl, pyridinyl, pyridazinyl, pyrimidinyl, pyrazinyl, pyrrolyl, pyrazolyl, oxazolyl, isoxazolyl, thiazolyl, isothiazolyl, imidazolyl, oxadiazolyl, thiadiazolyl, 1,2,4-triazine and 1,3,5-triazine.

Halogen represents fluoro, chloro, bromo, or iodo, preferably fluoro and chloro.

### EMBODIMENT OF THE INVENTION

The compound of the formula (I) of the present invention can be, but not limited to be, prepared by combining various known methods. In some embodiments, one or more of the substituents, such as amino group, carboxyl group, and hydroxyl group of the compounds used as starting materials or intermediates are advantageously protected by a protecting group known to those skilled in the art. Examples of the protecting groups are described in “Protective Groups in Organic Synthesis (3rd Edition)” by Greene and Wuts, John Wiley and Sons, New York 1999.

The compound of the formula (I) of the present invention can be, but not limited to be, prepared by the Method [A], [B], [C] or [D] below.
Method [A]

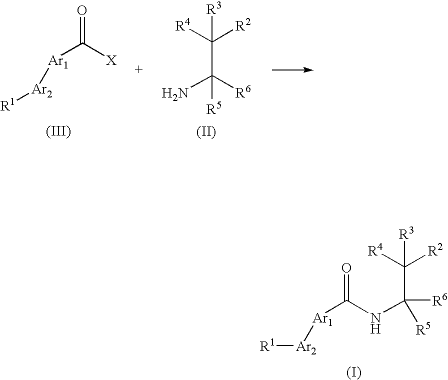

The compound of the formula (I) (wherein Ar₁, Ar₂, R¹, R², R³, R⁴, R⁵ and R⁶ are the same as defined above) can be obtained by the reaction of the compound of the formula (II) (wherein R², R³, R⁴, R⁵ and R⁶ are the same as defined above) with the compound of the formula (III) (wherein Ar₁, Ar₂, and R¹ are the same as defined above and X represents hydroxy or a salt thereof, halogen and the like)

The reaction may be carried out in a solvent including, for instance, halogenated hydrocarbons such as dichloromethane, chloroform and 1,2-dichloroethane; ethers such as diethyl ether, isopropyl ether, dioxane and tetrahydrofuran (THF) and 1,2-dimethoxyethane; aromatic hydrocarbons such as benzene, toluene and xylene; nitrites such as acetonitrile; amides such as N,N-dimethylformamide (DMF), N,N-dimethylacetamide (DMAC) and N-methylpyrrolidone (NMP); urea such as 1,3-dimethyl-2-imidazolidinone (DMI); sulfoxides such as dimethylsulfoxide (DMSO); and others. Optionally, two or more of the solvents selected from the listed above can be mixed and used.

The reaction can be advantageously carried out in the presence of a base including, for instance, organic amines such as pyridine, triethylamine and N,N-diisopropylethylamine, dimethylaniline, diethylaniline, 4-dimethylaminopyridine, and others and also can be advantageously carried out using coupling agent including, for instance, hydroxybenzotriazole, carbodiimides such as N,N-dicyclohexylcarbodiimide and 1-(3-dimethyl-aminopropyl)-3-ethylcarbodiimide; carbonyldiazoles such as 1,1′-carbonyldi(1,3-imiazole)(CDI) and 1,1′-carbonyldi(1,2,4-triazole)(CDT), and the like.

The reaction temperature can be optionally set depending on the compounds to be reacted. The reaction temperature is usually, but not limited to, about 0° C. to 50° C. The reaction may be conducted for, usually, 30 minutes to 48 hours and preferably 1 to 24 hours.

The compound of the formula (II) is commercially available or can be prepared by the use of known techniques.
Method [B]

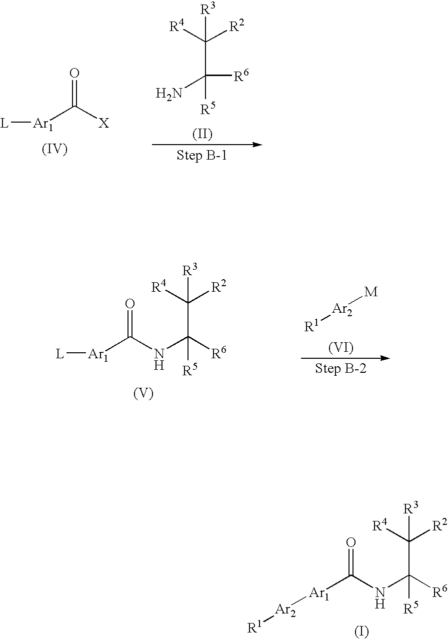

The compound of the formula (I) (wherein Ar₁, Ar₂, R¹, R², R³, R⁴, R⁵ and R⁶ are the same as defined above) can be obtained in two steps;

Step B-1: The compound of the formula (V) (wherein Ar₁, R², R³, R⁴, R⁵ and R⁶ are the same as defined above and L represents a leaving group including, for example, halogen atom such as chlorine, bromine, or iodine atom; and C₁₋₄ alkylsulfonyloxy group, e.g., trifluoromethane-sulfonyloxy, methanesulfonyloxy and the like) can be obtained by the reaction of the compound of the formula (IV) (wherein X, Ar₁ and L are the same as defined above) with the compound of the formula (II) (wherein R², R³, R⁴, R⁵and R⁶ are the same as defined above) in the same manner described in Method [A] for the preparation of the compound of the formula (I).

Step B-2: The compound of the formula (I) (wherein Ar₁, Ar₂, R¹, R², R³, R⁴, R⁵ and R⁶ are the same as defined above) can be obtained by the reaction of the compound of the formula (V) (wherein L, Ar₁, R², R³, R⁴, R⁵ and R⁶ are the same as defined above) with the compound of the formula (VI) (wherein Ar₂ and R¹ are the same as defined above and M represents metal group including, for instance, organoborane group such as boronic acid and di-methoxy boryl; organostannyl group such as tributyl stannyl, and the like) in the presence of a palladium catalyst such as tetrakis(triphenylphosphine)palladium.

The reaction can be advantageously carried out in the presence of a base including, for instance, cesium carbonate, sodium carbonate, potassium carbonate, potassium fluoride, sodium fluoride, and the like.

The reaction may be carried out in a solvent including, for instance, ethers such as diethyl ether, isopropyl ether, dioxane and tetrahydrofuran (THF) and 1,2-dimethoxyethane; aromatic hydrocarbons such as benzene, toluene and xylene; amides such as N,N-dimethylformamide (DMF), N,N-dimethylacetamide and N-methylpyrrolidone; sulfoxides such as dimethylsulfoxide (DMSO); alcohols such as methanol, ethanol, 1-propanol, isopropanol and tert-butanol; water and others. Optionally, two or more of the solvents selected from the listed above can be mixed and used.

The reaction temperature can be optionally set depending on the compounds to be reacted. The reaction temperature is usually, but not limited to, about 0° C. to 120° C. The reaction may be conducted for, usually, 30 minutes to 48 hours and preferably 1 to 24 hours.

The compound of the formula (IV) and (VI) are commercially available or can be prepared by the use of known techniques.
Method [C]

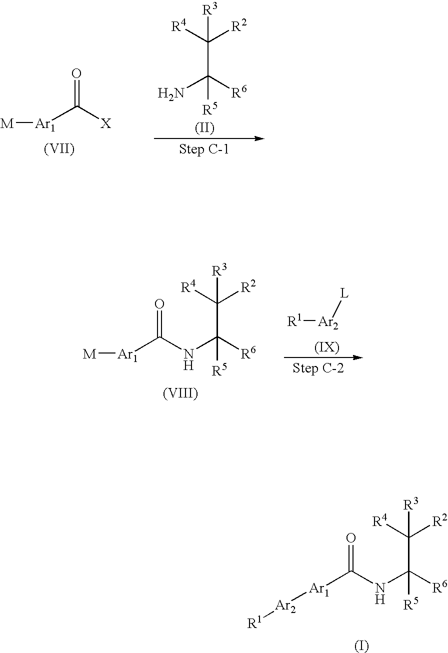

The compound of the formula (I) (wherein Ar₁, Ar₂, R¹, R², R³, R⁴, R⁵ and R⁶ are the same as defined above) can be obtained by in two steps;

Step C-1: The compound of the formula (VIII) (wherein M, Ar₁, R², R³, R⁴, R⁵ and R⁶ are the same as defined above) can be obtained by the reaction of the compound of the formula (VII) (wherein X, Ar₁ and M are the same as defined above) with the compound of the formula (II) (wherein R², R³, R⁴, R⁵and R⁶ are the same as defined above) in the same manner described in Method [A] for the preparation of the compound of the formula (I).

Step C-2: The compound of the formula (1) (wherein Ar₁, Ar₂, R¹, R², R³, R⁴, R⁵ and R⁶ are the same as defined above) can be obtained by the reaction of the compound of the formula (VIII) (wherein M, Ar₁, R², R³, R⁴, R⁵ and R⁶ are the same as defined above) with the compound of the formula (IX) (wherein L, Ar₂ and R¹ are the same as defined above) in the same manner described in Step B-2 of Method [B] for the preparation of the compound of the formula (I).

The compound of the formula (VII) and (IX) are commercially available or can be prepared by the use of known techniques.
Method [D]

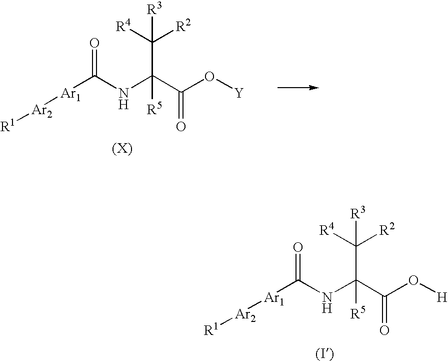

The compound of the formula (I′) (wherein Ar₁, Ar₂, R¹, R², R³, R⁴,and R⁵ are the same as defined above) can be obtained by the hydrolysis of the compound of formula (X) (wherein Ar₁, Ar₂, R¹, R², R³, R⁴,and R⁵ are the same as defined above, and Y represents C₁₋₆ alkyl).

The reaction can be advantageously carried out in the presence of a base including, for instance, alkali metal hydroxide such as sodium hydroxide, lithium hydroxide and potassium hydroxide; and others.
The reaction may be carried out in a solvent including, for instance, halogenated hydrocarbons such as dichloromethane, chloroform and 1,2-dichloroethane; ethers such as diethyl ether, isopropyl ether, dioxane and tetrahydrofuran (THF) and 1,2-dimethoxyethane; aromatic hydrocarbons such as benzene, toluene and xylene; amides such as N,N-dimethylformamide (DMF), N,N-dimethylacetamide and N-methylpyrrolidone; sulfoxides such as dimethylsulfoxide (DMSO); alcohols such as methanol, ethanol, 1-propanol, isopropanol and tert-butanol; water, and others. Optionally, two or more of the solvents selected from the listed above can be mixed and used.

The reaction temperature can be optionally set depending on the compounds to be reacted. The reaction temperature is usually, but not limited to, about 20° C. to 100° C. The reaction may be conducted for, usually, 30 minutes to 48 hours and preferably 1 to 24 hours.

The compound of the formula (X) can be, but not limited to be, prepared by the use of method [A], [B] or [C].

Preparation of the Intermediate

Preparation of the Formula (III′)

Method [E]

The compound of the formula (III′) (wherein Ar₁, Ar₂, and R¹ are the same as defined above and X′ represents hydroxy or salt form thereof) can be, but not limited to be, prepared by the following procedures.

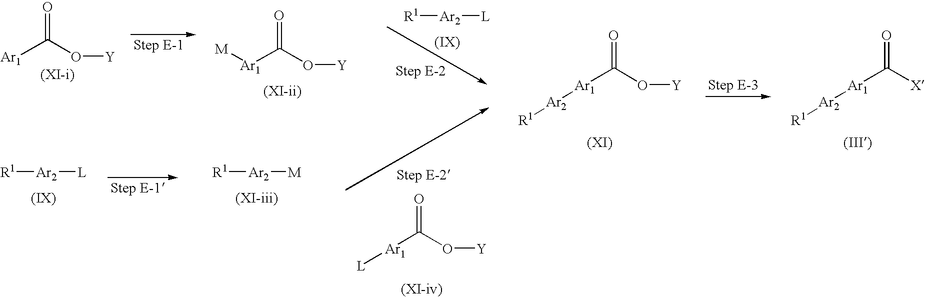

Step E-1: The compound of the formula (X-ii) (wherein Ar₁ is the same as defined above, M represents metal group including, for instance, organoborane group such as boronic acid and di-methoxy boryl; organostannyl group such as tributyl stannyl, and the like, and Y represents C₁₋₆ alkyl) can be obtained by the reaction of the compound of the formula (XI-i) (wherein Ar₁ and Y are the same as defined above) with a suitable organometallic reagent such as organoborane or organotin (e.g., tributyltin chloride, trimethyltin chloride, and the like).

The reaction can be advantageously carried out -in the presence of a base including, for instance, lithium diisopropylamine, lithium hexamethyldisilazide, organic amines such as pyridine, triethylamine and N,N-diisopropylethylamine, dimethylaniline, diethylaniline, 4-dimethylamino-pyridine, and others.
The reaction may be carried out in a solvent including, for instance, ethers such as diethyl ether, isopropyl ether, dioxane and tetrahydrofuran (THF) and 1,2-dimethoxyethane; aromatic hydrocarbons such as benzene, toluene and ethylbenzen; aliphatic hydrocarbons include hexane, heptane and octane; amides such as N,N-dimethylformamide (DMF), N,N-dimethylacetamide and N-methylpyrrolidone; sulfoxides such as dimethylsulfoxide (DMSO); alcohols such as methanol, ethanol, 1-propanol, isopropanol and tert-butanol and others. Optionally, two or more of the solvents selected from the listed above can be mixed and used.

The reaction temperature can be optionally set depending on the compounds to be reacted. The reaction temperature is usually, but not limited to, about −100° C. to 100° C. The reaction may be conducted for, usually, 30 minutes to 48 hours and preferably 1 to 24 hours.

Step E-2: The compound of the formula (XI) (wherein Ar₁, Ar₂, R¹ and Y are the same as defined above) can be obtained by the reaction of the compound of the formula (IX) (wherein Ar₂, R¹ and L are the same as defined) with the compound of the formula (XI-ii) (wherein Ar₁, Y and M are the same as defined above) in the same manner described in Step B-2 of Method [B] for the preparation of the compound of the formula (I).

Step E -1′: The compound of the formula (XI-iii) (wherein Ar₂, R¹ and M are the same as defined above) can be obtained by the reaction of the compound of the formula (IX) (wherein Ar₂, R¹ and L are the same as defined) in the same manner described in Step E-1 of Method [E] for the preparation of the compound of the formula (XI-ii).

Step E-2′: The compound of the formula (XI) (wherein Ar₁, Ar₂, R¹ and Y are the same as defined above) can be obtained by the reaction of the compound of the formula (XI-iii) (wherein Ar₂, R¹ and M are the same as defined) with the compound of the formula (XI-iv) (wherein Ar₁, L and Y are the same as defined) in the same manner described in Step B-2 of Method [B] for the preparation of the compound of the formula (I).

Step E-3: The compound of the formula (III′) (wherein Ar₁, Ar₂, R¹ and X′ are the same as defined above) can be obtained by the reaction of the compound of the formula (XI) (wherein Ar₁, Ar₂, R¹ and Y are the same as defined above) in the same manner described in Method [D] for the preparation of the compound of the formula (I′).

The compound of the formula (XI-i) and (XI-iv) are commercially available or can be prepared by the use of known techniques.

Preparation of the Formula (III″)

Method [F]

The compound of formula (III″) (wherein Ar₂, and R¹ are the same as defined above, Y represents C₁₋₆ alkyl and Q represents O or NH) can be, but not limited to be, prepared in two steps. 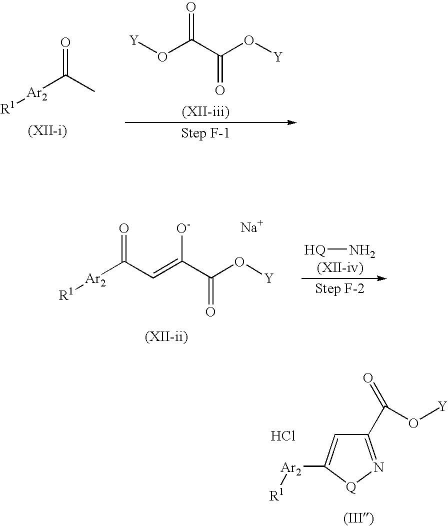
Step F-1: The compound of the formula (XII-ii) (wherein Ar₂, R¹ and Y are the same as defined above) can be obtained by the reaction of the compound of formula (XII-i) (wherein Ar₂ and R¹ are the same as defined above) with the compound of formula (XII-iii) (wherein Y is the same as defined above).
The reaction may be cared out in a solvent including, for instance, halogenated hydrocarbons such as dichloromethane, chloroform and 1,2-dichloroethane; ethers such as diethyl ether, isopropyl ether, dioxane and tetrahydrofuran (THF) and 1,2-dimethoxyethane; aromatic hydrocarbons such as benzene, toluene and xylene; amides such as N,N-dimethylformamide DMF), N,N-dimethylacetamide and N-methylpyrrolidone; sulfoxides such as dimethylsulfoxide (DMSO); and others. Optionally, two or more of the solvents selected from the listed above can be mixed and used.

The reaction can be advantageously carried out in the presence of a base including, for instance, cesium carbonate, sodium carbonate, potassium carbonate, potassium fluoride, sodium fluoride, triethylamine, sodium hydride, and the like.

The reaction temperature can be optionally set depending on the compounds to be reacted. The reaction temperature is usually, but not limited to, about 0° C. to 120° C.

The reaction may be conducted for, usually, 30 minutes to 48 hours and preferably 1 to 10 hours.

Step F-2: The compound of the formula (III″) (wherein Ar₂, R¹, Y and Q are the same as defined above) can be obtained by the reaction of the compound of formula (XII-ii) (wherein Ar₂, R¹, and Y are the same as defined above) with the compound of the formula (XII-iv) (wherein Q is the same as defined above).
The reaction may be carried out in a solvent including, for instance, ethers such as diethyl ether, isopropyl ether, dioxane and tetrahydrofuran (THF) and 1,2-dimethoxyethane; aromatic hydrocarbons such as benzene, toluene and xylene; amides such as N,N-di-methylformamide (DMF), N,N-dimethylacetamide and N-methylpyrrolidone; sulfoxides such as dimethylsulfoxide (DMSO); alcohols such as methanol, ethanol, 1-propanol, isopropanol and tert-butanol and others. Optionally, two or more of the solvents selected from the listed above can be mixed and used.

The reaction temperature can be optionally set depending on the compounds to be reacted. The reaction temperature is usually, but not limited to, about 20° C. to 100° C. The reaction may be conducted for, usually, 30 minutes to 48 hours and preferably 1 to 10 hours.

The compound of the formula (XII-i) (XII-iii), and (XII-iv), are commercially available or can be prepared by the use of known techniques.

When the compound shown by the formula (I) or a salt thereof has an asymmetric carbon in the structure, their optically active compounds and racemic mixtures are also included in the scope of the present invention.

Typical salts of the compound shown by the formula (I) include salts prepared by reaction of the compounds of the present invention with a mineral or organic acid, or an organic or inorganic base. Such salts are known as acid addition and base addition salts, successively.

Acids to form salts include inorganic acids such as, without limitation, sulfuric acid, phosphoric acid, hydrochloric acid, hydrobromic acid, hydriodic acid and the like, and organic acids, such as, without limitation, p-toluenesulfonic acid, methanesulfonic acid, oxalic acid, p-bromophenyl-sulfonic acid, succinic acid, citric acid, benzoic acid, acetic acid, and the like.

Base addition salts include those derived from inorganic bases, such as, without limitation, ammonium hydroxide, alkaline metal hydroxide, alkaline earth metal hydroxides, carbonates, bicarbonates, and the like, and organic bases, such as, without limitation, ethanolamine, triethylamine, tris(hydroxymethyl)aminomethane, and the like. Examples of inorganic bases include, sodium hydroxide, potassium hydroxide, potassium carbonate, sodium carbonate, sodium bicarbonate, potassium bicarbonate, calcium hydroxide, calcium carbonate, and the like.

The compound of the present invention or a salts thereof, depending on its substituents, may be modified to form lower alkylesters or known other esters; and/or hydrates or other solvates. Those esters, hydrates, and solvates are included in the scope of the present invention.

The compound of the present invention may be administered in oral forms, such as, without limitation normal and enteric coated tablets, capsules, pills, powders, granules, elixirs, tinctures, solution, suspensions, syrups, solid and liquid aerosols and emulsions. They may also be administered in parenteral forms, such as, without limitation, intravenous, intraperitoneal, subcutaneous, intramuscular, and the like forms, well known to those of ordinary skill in the pharmaceutical arts. The compounds of the present invention can be administered in intranasal form via topical use of suitable intranasal vehicles, or via transdermal routes, using transdermal delivery systems well known to those of ordinary skilled in the art.

The dosage regimen with the use of the compounds of the present invention is selected by one of ordinary skill in the arts, in view of a variety of factors, including, without limitation, age, weight, sex, and medical condition of the recipient, the severity of the condition to be treated, the route of administration, the level of metabolic and excretory function of the recipient, the dosage form employed, the particular compound and salt thereof employed.

The compounds of the present invention are preferably formulated prior to administration together with one or more pharmaceutically acceptable excipients. Excipients are inert substances such as, without limitation carriers, diluents, flavoring agents, sweeteners, lubricants, solubilizers, suspending agents, binders, tablet disintegrating agents and encapsulating material.

Yet another embodiment of the present invention is pharmaceutical formulation comprising a compound of the invention and one or more pharmaceutically acceptable excipients that are compatible with the other ingredients of the formulation and not deleterious to the recipient thereof. Pharmaceutical formulations of the invention are prepared by combining a therapeutically effective amount of the compounds of the invention together with one or more pharmaceutically acceptable excipients. In making the compositions of the present invention, the active ingredient may be mixed with a diluent, or enclosed within a carrier, which may be in the form of a capsule, sachet, paper, or other container. The carrier may serve as a diluent, which may be solid, semi-solid, or liquid material which acts as a vehicle, or can be in the form of tablets, pills, powders, lozenges, elixirs, suspensions, emulsions, solutions, syrups, aerosols, ointments, containing, for example, up to 10% by weight of the active compound, soft and hard gelatin capsules, suppositories, sterile injectable solutions and sterile packaged powders.

For oral administration, the active ingredient may be combined with an oral, and non-toxic, pharmaceutically-acceptable carrier, such as, without limitation, lactose, starch, sucrose, glucose, sodium carbonate, mannitol, sorbitol, calcium carbonate, calcium phosphate, calcium sulfate, methyl cellulose, and the like; together with, optionally, disintegrating agents, such as, without limitation, maize, starch, methyl cellulose, agar bentonite, xanthan gum, alginic acid, and the like; and optionally, binding agents, for example, without limitation, gelatin, natural sugars, beta-lactose, corn sweeteners, natural and synthetic gums, acacia, tragacanth, sodium alginate, carboxymethylcellulose, polyethylene glycol, waxes, and the like; and, optionally, lubricating agents, for example, without limitation, magnesium stearate, sodium stearate, stearic acid, sodium oleate, sodium benzoate, sodium acetate, sodium chloride, talc, and the like.

In powder forms, the carrier may be a finely divided solid which is in admixture with the finely divided active ingredient. The active ingredient may be mixed with a carrier having binding properties in suitable proportions and compacted in the shape and size desired to produce tablets. The powders and tablets preferably contain from about 1 to about 99 weight percent of the active ingredient which is the novel composition of the present invention. Suitable solid carriers are magnesium carboxymethyl cellulose, low melting waxes, and cocoa butter.

Sterile liquid formulations include suspensions, emulsions, syrups and elixirs. The active ingredient can be dissolved or suspended in a pharmaceutically acceptable carrier, such as sterile water, sterile organic solvent, or a mixture of both sterile water and sterile organic solvent.

The active ingredient can also be dissolved in a suitable organic solvent, for example, aqueous propylene glycol. Other compositions can be made by dispersing the finely divided active ingredient in aqueous starch or sodium carboxymethyl cellulose solution or in suitable oil.

The formulation may be in unit dosage form, which is a physically discrete unit containing a unit dose, suitable for administration in human or other mammals. A unit dosage form can be a capsule or tablets, or a number of capsules or tablets. A “unit dose” is a predetermined quantity of the active compound of the present invention, calculated to produce the desired therapeutic effect, in association with one or more excipients. The quantity of active ingredient in a unit dose may be varied or adjusted from about 0.1 to about 1000 milligrams or more according to the particular treatment involved.

Typical oral dosages of the present invention, when used for the indicated effects, will range from about 0.01 mg/kg/day to about 100 mg/kg/day, preferably from 0.1 mg/kg/day to 30 mg/kg/day, and most preferably from about 0.5 mg/kg/day to about 10 mg/kg/day. In the case of parenteral administration, it has generally proven advantageous to administer quantities of about 0.001 to 100 mg/kg/day, preferably from 0.01 mg/kg/day to 1 mg/kg/day. The compounds of the present invention may be administered in a single daily dose, or the total daily dose may be administered in divided doses, two, three, or more times per day. Where delivery is via transdermal forms, of course, administration is continuous.

### EXAMPLES

The present invention will be described in detail below in the form of examples, but they should by no means be construed as defining the meets and bounds of the present invention.

In the examples below, all quantitative data, if not stated otherwise, relate to percentages by weight.

Melting points are uncorrected. Liquid Chromatography-Mass spectroscopy (LC-MS) data were recorded on a Micromass Platform LC with Shimadzu Phenomenex ODS column (4.6 mm×30 mm) flushing a mixture of acetonitrile-water (9:1 to 1:9) at 1 ml/min of the flow rate. Mass spectra were obtained using electrospray (ES) ionization techniques (micromass Platform LC). TLC was performed on a precoated silica gel plate (Merck silica gel 60 F-254). Silica gel (WAKO-gel C-200 (75-150 μm)) was used for all column chromatography separations. All chemicals were reagent grade and were purchased from Sigma-Aldrich, Wako pure chemical industries, Ltd., Great Britain, Tokyo kasei kogyo Co., Ltd., Japan, Nacalai tesque, Inc., Watanabe Chemical Ind. Ltd., Maybridge plc, Lancaster Synthesis Ltd., Great Britain, Merck KgaA, Germany, Kanto Chemical Co., Ltd. ¹H NMR spectra were recorded using either Bruker-DRX-300 (300 MHz for ¹H) spectrometer or Brucker 500 UltraShieled™ (500 MHz for 1H). Chemical shifts are reported in parts per million (ppm) with tetramethylsilane (TMS) as an internal standard at zero ppm. Coupling constant (J) are given in hertz and the abbreviations s, d, t, q, m, and br refer to singlet, doblet, triplet, quartet, multiplet, and broad, respectively. The mass determinations were carried out by MAT95 (Finnigan MAT).

The effects of the present compounds were examined by the following assays and pharmacological tests.

[Measurement of the [³H]-Iloprost Binding to HEL Cells] (Assay 1)

A human erythloleukemia cell line, HEL 92.1.7, was purchased from American Type Culture Correction and maintained in RPMI-1640 medium (Gibco BRL) supplemented with 10% fetal calf serum (FCS), 2 mM glutamine, 4.5 g/L glucose, 10 mM Hepes, 1 mM sodium pyruvate, 100 U/ml penicillin, and 100 μg/ml streptomycin in a humidified 5% CO₂ atmosphere at 37° C. Cells were collected with centrifugation and washed with binding assay buffer (BAB: 50 mM Tris-HCl, 5 mM MgCl₂ (pH 7.5)). Cells were suspended at the density of 6.25×10⁶ cells/ml in BAB, and one million cells in 160 μl aliquot of cell suspension were put in a well of 96 well plate (Falcon). Then, 20 μl of compound solution, 100 μM of iloprost (for non-specific binding), or buffer alone (total binding), diluted with 1% DMSO in BAB was added. Finally, another 20 μl containing [³H]-iloprost (0.02 μCi, 0.5-1 pmol) in BAB was added and incubated at room temperature for 30 min with a gentle shaking. Cell suspension was then transferred to a well of MultiScreen plate with GF/C glass filters (Millipore) to harvest cells. Cells were washed twice with 200 μl of ice-cold BAB and the plate was kept at 55° C. for 30 min to dry filters. The filter in the well was punched out to a counting tube and 2 ml of Ultima Gold XR Packard) was added. [³H]-radio activity in the filter was measured by a liquid scintillation counter (Beckman, USA).

[Iloprost-Induced cAMP Production Assay in HEL Cells] (Assay 2)

HEL cells were collected with centrifugation and washed with cAMP assay buffer (CAB: Hank's balanced salt solution, 17 mM Hepes, 0.1% bovine serum albumin, 1 mM IBMX, 0.4% DMSO, and 1 mM L-ascorbic acid sodium salt (pH 7.4)). Cells were suspended at the density of 2.5×10⁵ cells/ml in CAB, and twenty thousand cells in 80 μl aliquot of cell suspension were put in a well of 96 well plate (Falcon). Then, 10 μl of compound solution diluted with 1% DMSO in CAB or buffer alone was added. The plate was incubated at 37° C. for 30 min. Then, another 10 μl containing 100 nM iloprost in CAB or buffer alone was added and further incubated at 37° C. for 30 min. cAMP content in the well was measured by, a cAMP ELISA kit (Applied Biosystems, USA).

[Measurement of Rhythmic Bladder Contraction in Anesthetized Rats]

(1) Animals

Female Sprague-Dawley Rats (200˜250 g/Charles River Japan) were Used.

(2) Rhythmic Bladder Contraction in Anesthetized Rats

Rats were anesthetized by intraperitoneal administration of urethane (Sigma) at 1.25 g/kg. The trachea was cannulated with a polyethylene tube (HIBIKI, No.8) to facilitate respiration; and a cannula (BECTON DICKINSON, PE-50) was placed in the left femoral vein for intravenous administration of testing compounds. The abdomen was opened through a midline incision, and after both ureters were cut, a water-filled balloon (about 1 ml capacity) was inserted through the apex of the bladder dome. The balloon was connected to a pressure transducer onto a polygraph. Rhythmic bladder contraction was elicited by raising up intravesical pressure to approximately 15 cm H₂O. After the rhythmic bladder contraction was stable, a testing compound was administered intravenously. Activity was estimated by measuring disappearance time and amplitude of the rhythmic bladder contraction. The effect on amplitude of bladder contractions was expressed as a percent suppression of the amplitude of those after the disappearance was recovered. Experimental values were expressed as the mean±S.E.M. The testing compound-mediated inhibition of the rhythmic bladder contraction was evaluated using Student's t-test A probability level less than 5% was accepted as significant difference.

Results of PGI2 receptor binding/cAMP is shown in Examples and tables of the Examples below. The data corresponds to the compounds as yielded by solid phase synthesis and thus to levels of purity of about 40 to 90%. For practical reasons, the compounds are grouped in three classes of activity as follows:

In vitro activity grade=A≦0.1 μM<B≦1 μM<C

The compounds of the present invention also show excellent selectivity, and strong activity in vivo assays.

Preparing Method of Starting Compounds

[Starting Compound 1A]

3-(4benzyloxyphenyl)-1-methoxycarbonyl-3-oxopropen-1-olate

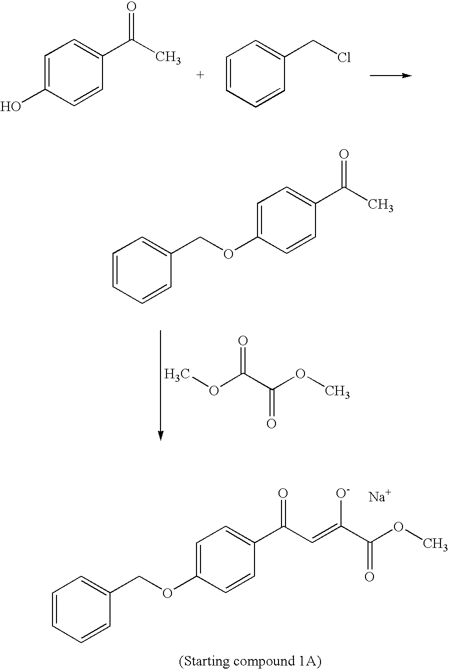

A mixture of 4-hydroxyacetophenone (13.3 g, 97.7 mmol) and benzylchloride (14.8 g, 13.5 ml, 117.2 mmol), sodium iodide (19.0 g, 127.0 mmol) and potassium carbonate (20.2 g, 146.5 mmol) in acetone (80 ml) was stirred under reflux for 5 hours. After the mixture was concentrated under reduced pressure, the residue was diluted with water, and extracted with ethyl acetate. The separated organic phase was washed with 1N HCl, 1N NaOH and brine, dried over sodium sulfate, filtered and concentrated under reduced pressure. The crude product was recrystallized from ethyl acetate to give 1-(4-benzyloxyphenyl)ethanone (15.3 g, 69%) as a colorless solid.

Next, to a suspension of sodium hydride (0.707 g, 60% in oil, 17.7 mmol) in toluene (20 mL) was added oxalic acid dimethyl ester (1.566 g, 13.26 mmol) followed by 1-(4-benzyloxyphenyl)ethanone (2.000 g, 8.839 mmol). The mixture was stirred at 60° C. for 3 hours. After cooled to room temperature, the mixture was diluted with water (30 mL). The resulting precipitate was collected by filtration, washed several times with water and ethyl acetate, dried under reduced pressure to give sodium 3-(4-benzyloxyphenyl)-1-methoxycarbonyl-3-oxopropen-1-olate (3.050 g, quant.) as a pale yellow solid.

### Example 1-1

### 5-(4-Benzyloxyphenyl)-1H-pyrazole-3-carboxylic acid methyl ester

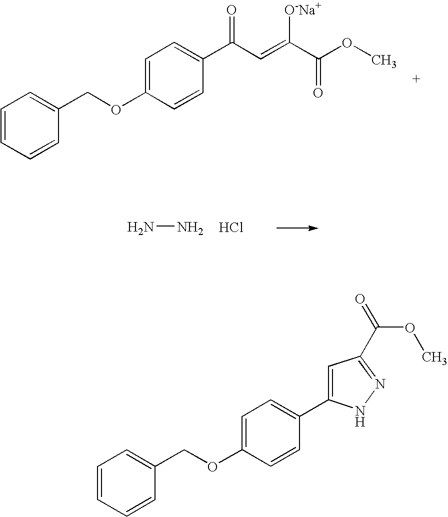

A mixture of sodium 3-(4-benzyloxyphenyl)-1-methoxycarbonyl-3-oxopropen-1-olate (starting compound 1A) (0.500 g, 1.496 mmol), hydrazine monohydrochloride (0.330 g, 4.82 mmol) and methanol (15 mL) was heated at reflux for 1 hour. After cooled to room temperature, the mixture was diluted with water (15 mL). The resulting precipitate was collected by filtration, washed with 50% methanol/water, and dried under reduced pressure to give 5-(4-benzyloxyphenyl)-1H-pyrazole-3-carboxylic acid methyl ester (0.324 g, 70%) as a colorless solid.
Lithium 5-(4-benzyloxyphenyl)-1H-pyrazole-3-carboxylate

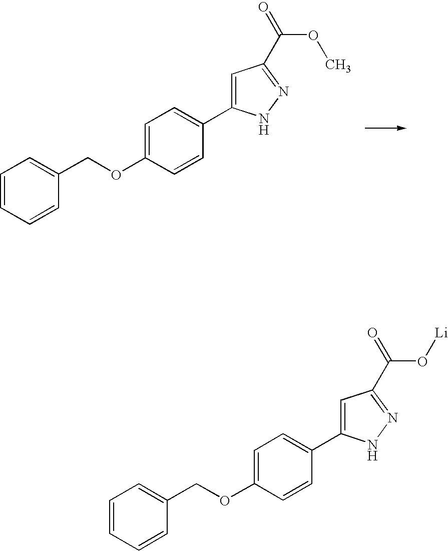

To a solution of 5-(4benzyloxyphenyl)-1H-pyrazole-3-carboxylic acid methyl ester (0.310 g, 1.01 mmol) in methanol (1.5 mL), water (1.5 mL) and THF (3 mL) was added lithium hydroxide monohydrate (0.118 g, 2.81 mmol). After 15 min, the mixture was stirred at 60° C. for 3 hours. After cooled to room temperature, the mixture was diluted with water(10 mL). The resulting precipitate was collected by filtration, washed with ether and water successively, and dried under reduced pressure to give lithium 5-(4-benzyloxyphenyl)-1H-pyrazole-3-carboxylate (0.191 g, 63%) as a colorless solid.
2-{[5-(4-Benzyloxyphenyl)-1H-pyrazole-3-carbonyl]amino}-3-phenylpropionic acid methyl ester

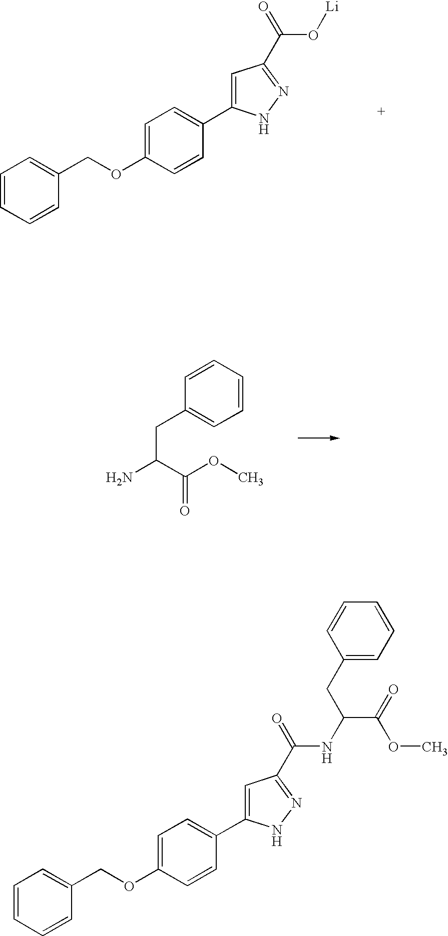

To a mixture of lithium 5-(4-benzyloxyphenyl)-1H-pyrazole-3-carboxylate (0.080 g, 0.266 mmol), phenylalanine methyl ester hydrochloride (0.069 g, 0.320 mmol), 1-hydroxybenzotriazol (0.054 g, 0.400 mmol), triethylamine (0.045 mL, 0.320 mmol) and DMF (3 mL) was added N-(3-dimethylaminopropyl)-N′-ethylcarbodiimide hydrochloride (0.077 g, 0.400 mmol), and the stirring was continued overnight. The mixture was diluted with water (10 mL). The resulting precipitate was collected by filtration, washed with water, and dried under reduced pressure to give 2-{[5-(4-benzyloxyphenyl)-1H-pyrazole-3-carbonyl]amino}-3-phenylpropionic acid methyl ester (0.109 g, 90%) as a colorless solid.
2-{[5-(4-Benzyloxyphenyl)-1H-pyrazole-3-carbonyl]amino}-3-phenylpropionic acid

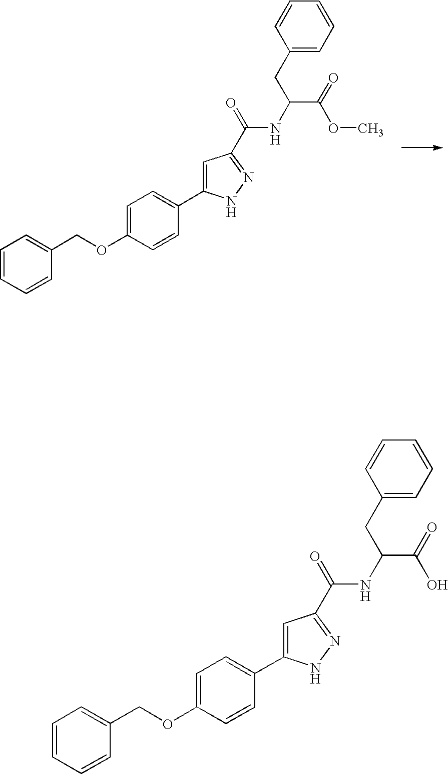

To a solution of 2-{[5-(4-benzyloxyphenyl)-1H-pyrazole-3-carbonyl]amino}-3-phenylpropionic acid methyl ester (0.084 g, 0.184 mmol) in methanol (1 mL), water (1 mL) and THF (2 mL) was added lithium hydroxide monohydrate (0.022 g, 0.524 mmol), and the stirring was continued for 2 hours. The reaction mixture was diluted with water (10 mL) and washed with ether. The separated aqueous phase was acidified to pH 2 by adding 1N HCl. The mixture was partitioned between ethyl acetate and water. The separated organic phase was washed with brine, dried over sodium sulfate, filtered and concentrated under reduced pressure. The residue was recrystallized from ethanol and diisopropyl ether to give 2-{[5-(4-benzyloxyphenyl)-1H-pyrazole-3-carbonyl]amino}-3-phenylpropionic acid (0.060 g, 77%) as a colorless solid.

Melting point: 196-200° C.

Molecular weight: 441;49

Mass spectrometry: 442 (M+H)⁺

In vitro activity grade: A

¹H-NMR (500 MHz, DMSO-d6): δ 3.13 (1H, br), 3.19 (1H, dd, J=4.9, 13.7 Hz), 4.65 (1H, dd, J=8.2, 13.7 Hz), 5.15 (2H, s), 6.96 (1H, br), 7.09 (1H, d, J=8.7 Hz), 7.18 (1H, ddd, J=4.4, 8.7, 8.8 Hz), 7.26 (4H, d, J=4.4 Hz), 7.34 (1H, t, J=7.3 Hz), 7.40 (2H, t, J=7.3 Hz), 7.46 (2H, d, J=7.3 Hz), 7.70 (2H; d, J=8.8 Hz), 7.98 (1H, br), 12.94 (1H, br), 13.47 (1H, br).

### Example 1-2

### 5-(4-Benzyloxyphenyl)isoxazole-3-carboxylic acid methyl ester

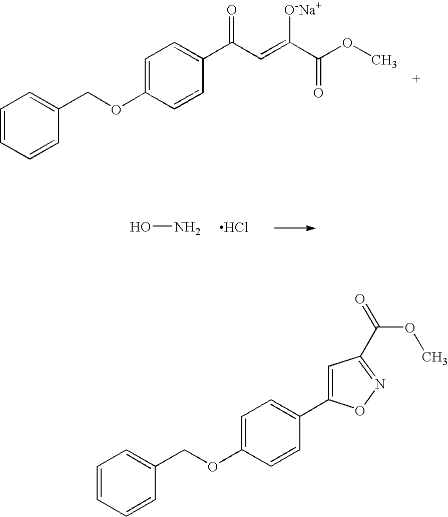

A mixture of sodium 3-(4-benzyloxyphenyl)-1-methoxycarbonyl-3oxopropen-1-olate (starting compound 1A) (0.500 g, 1.496 mmol), hydroxyammonium chloride (0.520 g, 7.478 mmol) and methanol (15 mL) was stirred at reflux for 5 hours. After the mixture was allowed to cool to r.t., a needle crystal was formed. The mixture was diluted with water (ca. 3 mL), and set aside for further 1 hour. The crystal was collected by filtration, washed with methanol and water, and dried in vacuo to give 5-(4-benzyloxyphenyl)isoxazole-3-carboxylic acid methyl ester (0.090 g, 20%) as a white needle.
5-(4-Benzyloxyphenyl)isoxazole-3-carboxylic acid

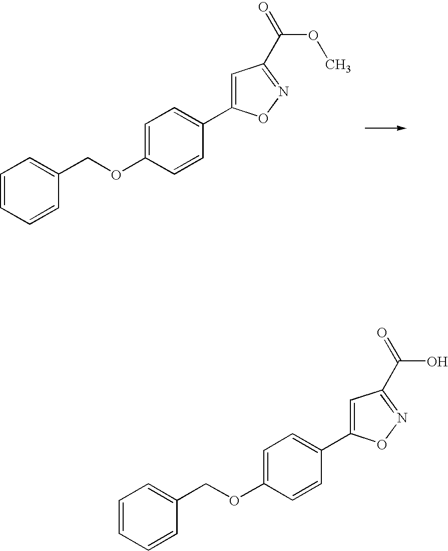

To a suspension of 5-(4-benzyloxyphenyl)isoxazole-3-carboxylic acid methyl ester (0.080 g, 0.26 mmol) in methanol (1 mL), water (1 mL) and THF (2 mL) was added lithium hydroxide monohydrate (0.019 g, 0.45 mmol), and the stirring was continued for 1.5 hours. The mixture was diluted with water, washed with ether, and acidified to pH 1 by adding 1N HCl (0.5 mL). The mixture was partitioned between ethyl acetate and water. The separated organic phase was washed with brine, dried over sodium sulfate, filtered and concentrated under reduced pressure to give 5-(4-benzyloxyphenyl)isoxazole-3-carboxylic acid (0.076 g, quant.) as a white solid.
2-{[5-(4-Benzyloxyphenyl)isoxazole-3-carbonyl]amino}-3-phenylpropionic acid methyl ester

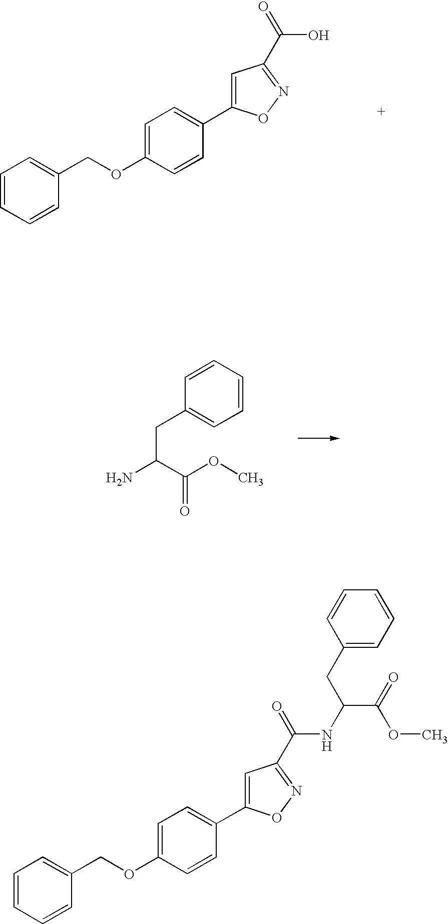

To a mixture of 5-(4-benzyloxyphenyl)isoxazole-3-carboxylic acid (0.070 g, 0.24 mmol), phenylalanine methyl ester hydrochloride (0.061 g, 0.28 mmol), 1-hydroxybenzotriazole (0.048 g, 0.36 mmol), triethylamine (0.040 mL, 0.28 mmol) and DMF (3 mL) was added N-(3-dimethylaminopropyl)-N′-ethylcarbodiimide hydrochloride (0.068 g, 0.36 mmol), and the mixture was stirred at room temperature overnight, and then diluted with water. The resultant precipitate was collected by filtration, washed with water and dried under reduced pressure. The crude product was purified by column chromatography on silica-gel (hexane:ethyl acetate, 3:1) to give 2-{[5-(4-benzyloxyphenyl)isoxazole-3-carbonyl]amino}-3-phenylpropionic acid methyl ester (0.100 g, 92%) as a white solid.
2-{[5-(4-Benzyloxyphenyl)isoxazole-3-carbonyl]amino}-3-phenyl-propionic acid

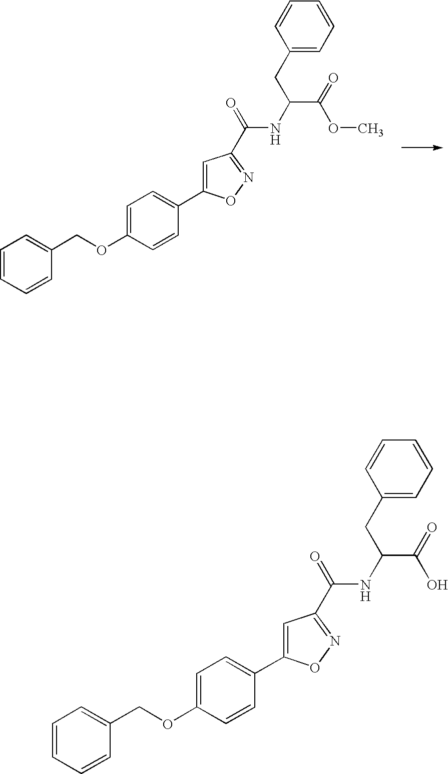

To a suspension of 2-{[5-(4-benzyloxyphenyl)isoxazole-3-carbonyl]amino}-3-phenylpropionic acid methyl ester (0.095 g, 0.21 mmol) in methanol (1.5 mL), water (1.5 mL) and THF (3 mL) was added lithiumhydroxide monohydrate (0.019, 0.45 mmol), and the mixture was stirred at room temperature for 2 hours. The mixture was acidified with 1N HCl (0.5 mL). The resultant crystal was collected by filtration, washed with water, and dried under reduced pressure to give 2-{[5-(4-benzyloxyphenyl)isoxazole-3-carbonyl]amino}-3-phenylpropionic acid (0.084 g, 91%) as a white solid.

Melting point: 172-173° C.

Molecular weight: 442.47

Mass spectrometry: 443 (M+H)⁺

In vitro activity grade: A

¹H-NMR (500 MHz, DMSO-d6): δ 3.12 (1H, dd, J=9.9, 14.0 Hz), 3.20 (1H, dd, J=4.6; 14.0 Hz), 4.62-4.67 (1H, m), 5.19 (2H, s), 7.15-7.21 (4H, m), 7.25-7.28 (4H, m), 7.34 (1H, t, J=7.3 Hz), 7.41 (2H, t, J=7.0 Hz), 7.46 (2H, d, J=7.0 Hz), 7.85 (2H, d, J=8.8 Hz), 8.85 (1H, d, J=8.2 Hz), 12.93 (1H, br).

### Example 2-1

### 5-Tributylstannanylthiophene-2-carboxylic acid methyl ester

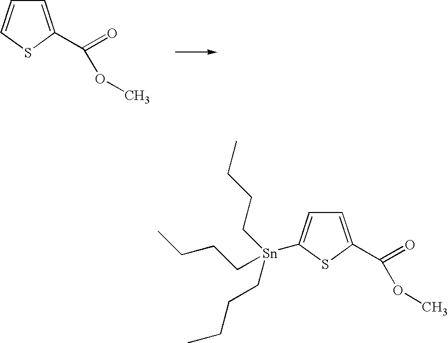

To a stirred solution of methyl thiophene-2-carboxylate (10.00 g, 70.34 mmol) in THF (100 mL) at −78° C. under an argon atmosphere was added dropwise a 2M solution of lithium diisopropylamine in heptanel/THF/ethylbenzene (42.0 mL, 84.0 mmol). After 1 hour, tributyltin chloride (21.0 mL, 77.4 mmol) was added dropwise. After being stirred at −78° C. for further 2 hours, the mixture was allowed to warm to room temperature, and the stirring was continued overnight. The resulting mixture was quenched by 1N HCl, and extracted with ethyl acetate. The organic phase was washed twice with water, dried over sodium sulfate, filtered, and concentrated under reduced pressure. The residue was purified by column chromatography on silica-gel (hexane:ethyl acetate, 30:1) to give 5-tributylstannanylthiophene-2-carboxylic acid methyl ester (8.40 g, 28%) as a slightly yellow oil.
5-(4-Benzyloxyphenyl)thiophene-2-carboxylic acid methyl ester

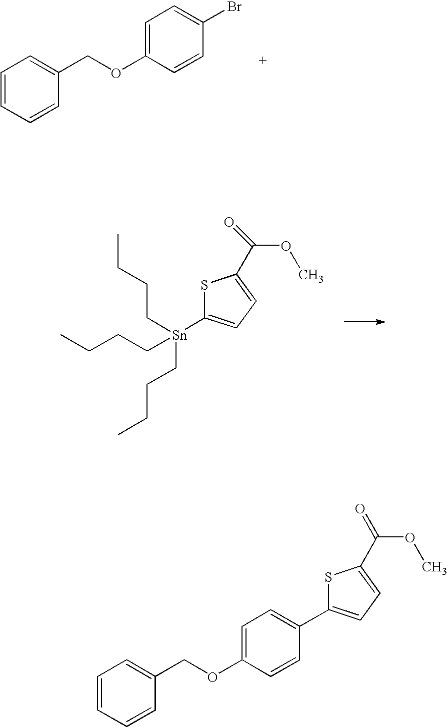

To a mixture of 4benzyloxybromobenzene (0.500 g, 1.90 mmol), 5-tributylstannanylthiophene-2-carboxylic acid methyl ester (starting compound 2A) (0.819 g, 1.90 mmol) and DMF (10 mL) under an argon atmosphere was added dichlorobis(triphenylphosphine)palladium (0.027 g, 0.038 mmol). The mixture was stirred at 100° C. overnight. After cooled to room temperature, the mixture was partitioned between ethyl acetate and saturated aqueous sodium bicarbonate solution. The separated organic phase was washed with water and brine, dried over sodium sulfate, filtered, and concentrated under reduced pressure. The residue was purified by column chromatography on silica gel (hexane:ethyl acetate, 15:1) to give 5-(4-benzyloxyphenyl)thiophene-2-carboxylic acid methyl ester (0.188 g, 31%) as a white solid.
5-(4-Benzyloxyphenyl)thiophene-2-carboxylic acid

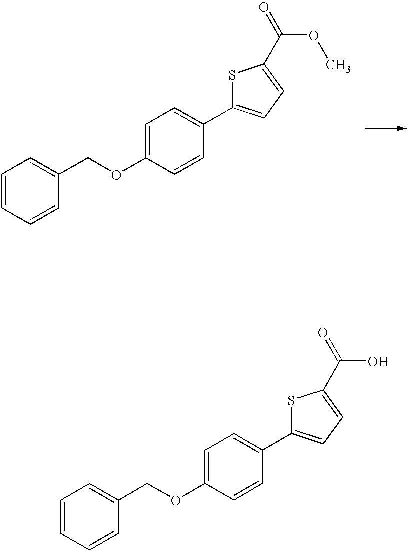

To a suspension of 5-(4-benzyloxyphenyl)thiophene-2-carboxylic acid methyl ester (0.125 g, 0.385 mmol) in methanol (2 mL), water (2 mL) and THF (2 mL) was added lithium hydroxide monohydrate (0.028 g, 0.67 mmol), and the mixture was stirred at 60° C. for 4 hours. After cooled to room temperature, the mixture was diluted with water and washed with ether. The separated aqueous phase was acidified with 1N HCl (1 mL). The resultant precipitate was collected by filtration, washed with water, and dried under reduced pressure to give 5-(4-benzyloxyphenyl)thiophene-2-carboxylic acid (0.089 g, 74%) as a pale yellow solid.
2-{[5-(4-Benzyloxyphenyl)thiophene-2-carbonyl]amino}-3-phenyl-propionic acid methyl ester

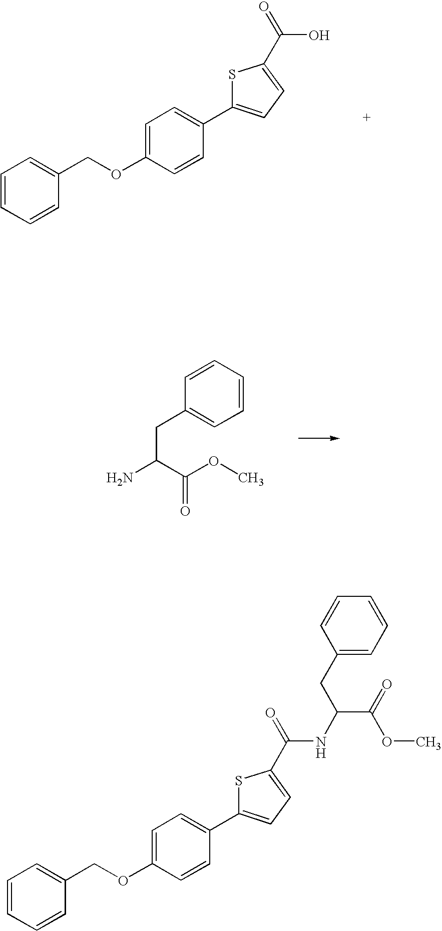

To a mixture of 5-(4-benzyloxyphenyl)thiophene-2-carboxylic acid (0.085 g, 0.27 mmol), phenylalanine methyl ester hydrochloride (0.089 g, 0.41 mmol), 1-hydroxybenzotriazole (0.074 g, 0.55 mmol), triethylamine (0.057 mL, 0.41 mmol) and DMF (3 mL) was added N-(3-dimethylaminopropyl)-N′-ethylcarbodiimide hydrochloride (0.079 g, 0.41 mmol), and the mixture was stirred at room temperature overnight. The mixture was diluted with water (ca. 3 mL). The resultant crystal was collected by filtration, washed with water and dried under reduced pressure. The crude product was purified by column chromatography on silica gel (hexane:ethyl acetate, 2:1) to give 2-{[5-(4-benzyloxyphenyl)thiophene-2-carbonyl]amino}-3-phenyl-propionic acid methyl ester (0.114 g, 88%) as a white solid
2-{[5-(4-Benzyloxyphenyl)thiophene-2-carbonyl]amino}-3-phenyl-propionic acid

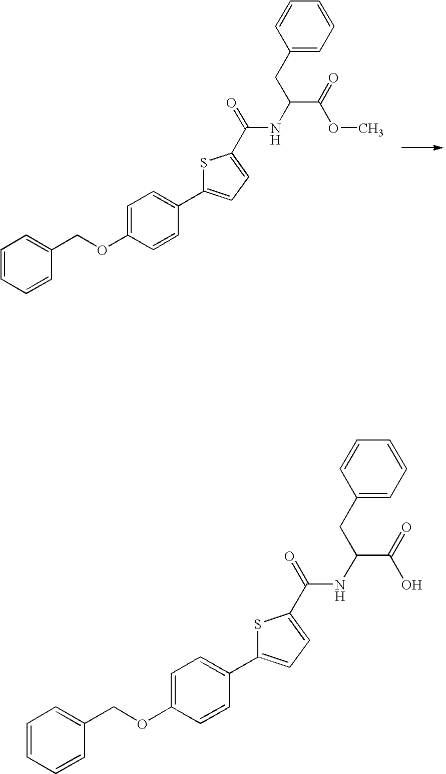

To a suspension of 2-{[5-(4-benzyloxyphenyl)thiophene-2-carbonyl]amino}-3-phenyl-propionic acid methyl ester (0.085 g, 0.18 mmol) in methanol (1 mL), water (1 mL) and THF (1 mL) was added lithium hydroxide monohydrate (0.013 g, 0.31 mmol), and the mixture was stirred at room temperature for 1 hour. The, mixture was diluted with water, and washed with ether. The separated aqueous phase was acidified with 1N HCl (0.5 mL). The resultant crystal was collected by filtration, washed with water, and dried under reduced pressure to give 2-{[5-(4-benzyloxyphenyl)thiophene-2-carbonyl]amino}-3-phenyl-propionic acid (0.074 g, 90%) as a white solid.

Melting point: 215-216° C.

Molecular weight: 457.55

Mass spectrometry: 458 (M+H⁺

In vitro activity grade: A

¹H-NMR (500 MHz, DMSO-d6): δ 3.04 (1H, dd, J=10.7, 13.9 Hz), 3.18 (1H, dd, J=4.4, 13.9 Hz), 4.57 (1H, ddd, J=2.2, 4.4, 8.2 Hz), 5.15 (2H, s), 7.07 (2H, d, J=8.8 Hz), 7.18 (1H, dd, J=6.9, 7.3 Hz), 7.25-7.35 (5H, m), 7.38-7.41 (3H, m), 7.45 (2H, d, J=7.3 Hz), 7.61 (2H, d, J=8.8 Hz), 7.77 (1H, d, J=3.8 Hz), 8.69 (1H, d, J=8.2 Hz), 12.81 (1H, br).

### Example 3-1

### 4′-Benzyloxy-biphenyl-4-carboxylic acid benzyl ester

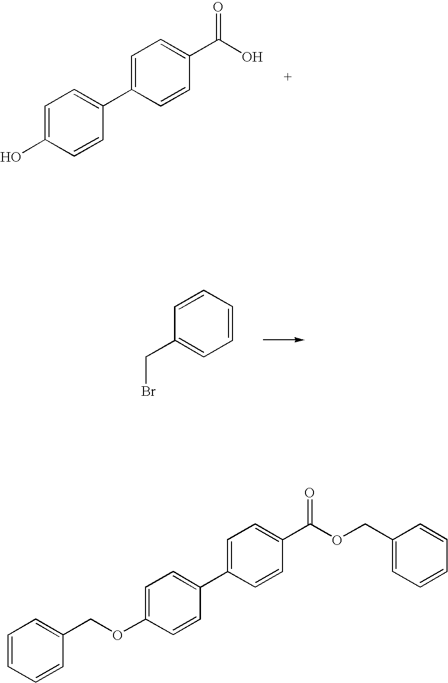

To a mixture of 4′-hydroxybiphenyl-4-carboxylic acid (1.000 g, 4.668 mmol), benzyl bromide (1.680 g, 9.822 mmol), sodium iodide (0.140 mg, 0.934 mmol) and DMF (5 mL) was added potassium carbonate (1.420 g, 10.27 mmol), and the mixture was stirred at 80° C. overnight. After cooled to room temperature, the mixture was diluted with ethyl acetate. The organic phase was washed with saturated aqueous ammonium chloride solution and brine, dried over magnesium sulfate, filtered and concentrated under reduced pressure to give 4′-benzyloxybiphenyl-4-carboxylic acid benzyl ester (1.130 g, 61%) as a white powder.
4′-Benzyloxybiphenyl-4-carboxylic acid

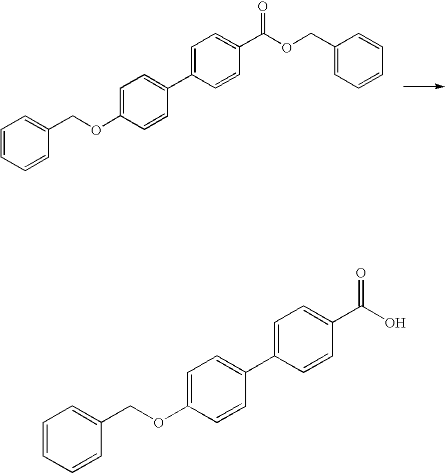

To a mixture of 4′-benzyloxybiphenyl-4-carboxylic acid benzyl ester (0.500 g, 1.27 mmol) and tetrahydrofuran was added 1N sodium hydroxide aqueous solution (5.00 mL, 5.00 mmol), and the mixture was stirred at reflux overnight. After cooled to room temperature, the precipitate was collected by filtration. The solid was suspended in water (10 mL) and acidified by 1N HCl aqueous solution (10.00 mL, 10.00 mmol). The suspension was filtered and washed with water. The collected solid was dried under reduced pressure to give 4′-benzyloxybiphenyl-4-carboxylic acid (0.251 g, 65%) as a white solid.
2-[(4′-Benzyloxybiphenyl-4-carbonyl)amino]-3-phenylpropionic acid methyl ester

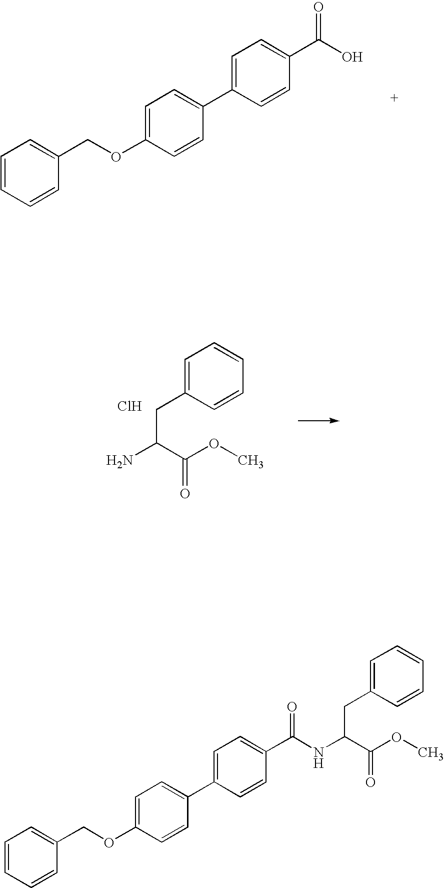

To a mixture of 4′-benzyloxybiphenyl-4-carboxylic acid (0.312 g, 1.03 mmol), phenylalanine methyl ester hydrochloride (0.184 mg, 1.03 mmol), 1-ethyl-3-(3-dimethylaminopropyl)carbodiimide hydrochloride (0.196 g, 1.03 mmol), 1-hydroxybenzotriazole (0.139 g, 1.03 mmol), DMF (5.0 mL) and dichloromethane(5.0 mL) was added triethylamine (0.104 g, 1.03 mmol), and the mixture was stirred at room temperature overnight. The mixture was partitionated between ethyl acetae and water. The separated organic phase was washed with saturated aqueous ammonium chloride solution and brine, dried over sodium sulfate, filtered and concentrated under reduced pressure. The crude product was purified by recrystallization from ethyl acetate and hexane to give 2-[(4′-benzyloxybiphenyl-4-carbonyl)amino]-3-phenylpropionic acid methyl ester (0.344 g, 72%) as a white solid.
2-[(4′-Benzyloxy-biphenyl-4-carbonyl)-amino]-3-phenyl-propionic acid

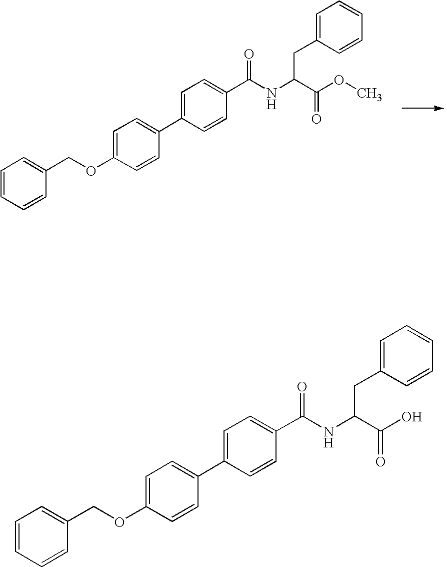

To a solution of 2-[(4′-benzyloxybiphenyl-4-carbonyl)amino]-3-phenylpropionic acid methyl ester (0.344 g, 0.739 mmol) in ethanol (5.0 mL), water (2.5 mL) and tetrahydrofuran (5.0 mL) was added lithium hydroxide (0.053 g, 2.2 mmol), and the mixture was stirred at room temperature for 2 hours. The precipitate was collected by filtration and washed with water. The solid was suspended in 1N HCl aqueous solution (2.00 mL, 2.00 mmol) and filtered on a filter. The solid was washed with water and dried under reduced pressure to give 2-[(4′-benzyloxybiphenyl-4-carbonyl)amino]-3-phenylpropionic acid (0.271 g, 81%) as a white solid.

Melting point: 302° C.

Molecular weight: 451.52

Mass spectrometry: 452 (M+H)⁺

In vitro activity grade: A

¹H-NMR (500 MHz, DMSO-d6): δ 3.09 (1H, dd, J=7.7, 13.4 Hz), 3.22 (1H, dd, J=4.7, 13.2 Hz), 4.37 (1H, m), 5.16 (2H, s), 7.10-7.13 (3H, m), 7.18-7.24 (4H, m), 7.32-7.35 (1H, m), 7.39-7.42 (2H, m), 7.47 (2H, d, J=7.3 Hz), 7.65-7.69 (4H, m), 7.78 (2H, d, J=8.2 Hz), 8.14 (1H, br s).

### Example 4-1

### 2-[(6-Chloropyridine-3-carbonyl)amino]-3-phenylpropionic acid methyl ester

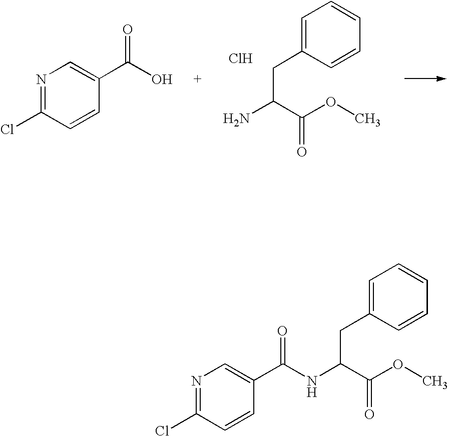

To a mixture of phenylalanine methyl ester hydrochloride (0.300 g, 1.39 mmol), 6-chloronicotinic acid (0.241 g, 1.53 mmol), 1-ethyl-3-(3-dimethylaminopropyl)carbodiimide hydrochloride (0.319 g, 1.67 mmol), 1-hydroxybenzotriazole (0.226 g, 1.67 mmol) and dichloromethane (10.0 mL) was added triethylamine (0.211 g, 2.09 mmol), and the mixture was stirred at room temperature overnight The mixture was concentrated under reduced pressure, and the reside was partitionated between ethyl acetate and water. The 'separated organic phase was washed with saturated aqueous ammonium chloride solution and brine, dried over sodium sulfate, filtered and concentrated under reduced pressure. The crude product was purified by column chromatography on silica-gel (chloroform:ethanol, 40:1) to give 2-[(6-chloropyridine-3-carbonyl)amino]-3-phenylpropionic acid methyl ester (0.468 g, quant.) as a pale yellow oil.
2-{[6-(4-benzyloxyphenyl)pyridine-3-carbonyl]amino}-3-phenylpropionic acid methyl ester

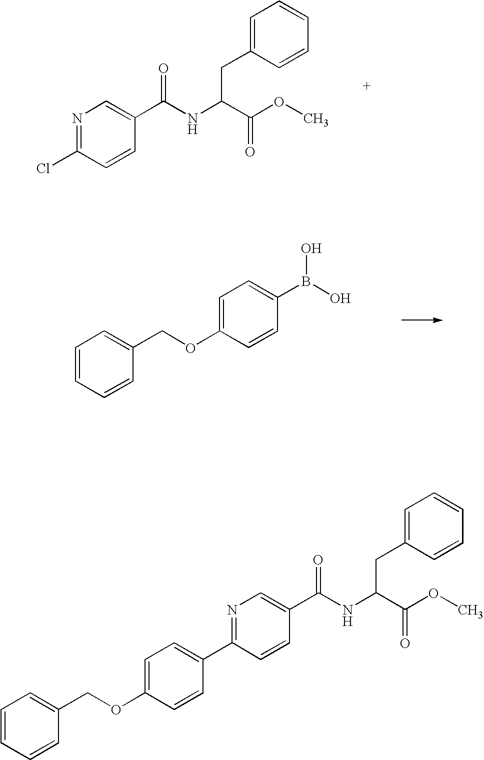

To a mixture of 2-[(6-chloropyridine-3-carbonyl)amino]-3-phenyl-propionic acid methyl ester (0.101 g, 0.317 mmol), 4-(benzyloxy)phenylboronic acid (0.079 g, 0.349 mmol) and 1,2-dimethoxyethane (2.0 mL) was added 1.9 N aqueous sodium carbonate solution (0.500 mL, 0.950 mmol) followed by tetrakis(triphenylphosphine)palladium (0.055 g, 0.048 mmol). The mixture was stirred at 90° C. for 4 hours. After cooled to room temperature, the mixture was partitioned between ethyl acetate and water. The separated organic phase was washed with brine, dried over sodium sulfate, filtered and concentrated under reduced pressure. The crude product was purified by preparative TLC (chloroform:ethanol, 40:1) to give 2-{[6-(4-benzyloxy-phenyl)pyridine-3-carbonyl]amino}-3-phenylpropionic acid methyl ester (0.0065 g, 4%) as a white solid.
2-{[6-(4-benzyloxy-phenyl)-pyridine-3-carbonyl]-amino}-3-phenyl-propionic acid

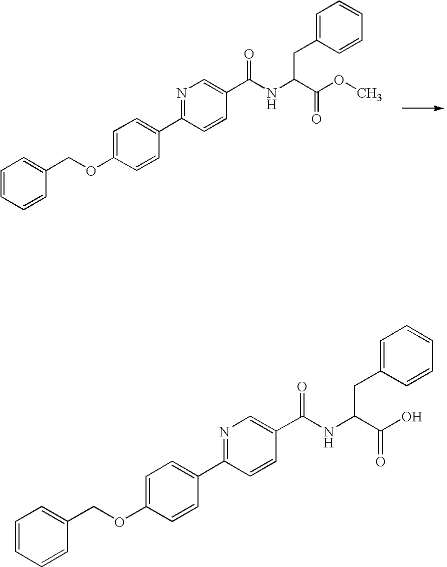

To a solution of 2-{[6-(4-Benzyloxyphenyl)pyridine-3-carbonyl]amino}-3-phenylpropionic acid methyl ester (0.0065 g, 0.014 mmol) in ethanol (0.250 mL), water (0.500 mL) and tetrahydrofuran (0.500 mL) was added 1N aqueous lithium hydroxide solution (0.160 mL, 0.160 mmol), and the mixture was stirred at room temperature for 3 hours. The mixture was acidified by 1 N aqueous HCl solution. The precipitate was collected by filtration and washed with water. The solid was dried under reduced pressure. The solid was suspended in ethyl acetate, filtered by filtration, and dried under reduced pressure to give 2-{[6-(4-benzyloxyphenyl)pyridine-3-carbonyl]amino}-3-phenyl-propionic acid (0.0023 mg, 37%) as a white solid.

Melting point: 240° C.

Molecular weight: 452.51

Mass spectrometry: 453 (M+H)⁺

In vitro activity grade: A

¹H-NMR (500 MHz, MeOD-d4): δ 3.10-3.14 (1H, m), 3.31-3.38 (1H, m), 4.82-4.90 (1H, m), 5.16 (2H, s), 7.10-7.12 (2H, m), 7.18-7.22 (1H, m), 7.28-7.33 (5H, m), 7.36-7.39 (2H, m), 7.45 (2H, d, J=7.6 Hz), 7.85 (1H, d, J=8.5 Hz), 7.97-7.99 (2H, m), 8.12 (1H, dd, J=2.3, 8.5 Hz), 8.67 (1H, s).

### Example 5-1

### 2-Benzyloxy-5-bromo-pyrimidine

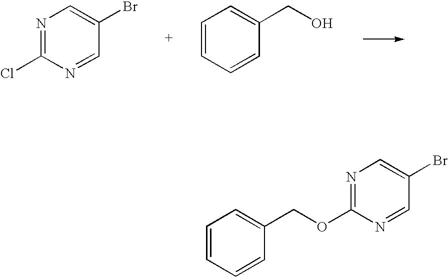

To a mixture of benzyl alcohol (0.123 g, 1.14 mmol) and tetrahedrofuran (12.0 mL) was added sodium hydride (60% oil suspension, 0.050 g, 1.2 mmol) portionwise. After 10 min, 5-bromo-2-chloropyrimidine (0.200 g, 1.034 mmol) was added, and the mixture was stirred at room temperature overnight. The mixture was concentrated under reduced pressure. The residue was partitionated between ethyl acetate and water. The separated organic phase was washed with brine, dried over sodium sulfate, filtered and concentrated under reduced pressure to give 2-benzyloxy-5-bromopyrimidine (0.269 g, 98%) as a white solid.

### 4-[N-(1-methoxycarbonyl-2-phenylethyl)carbamoyl]phenylboronic acid

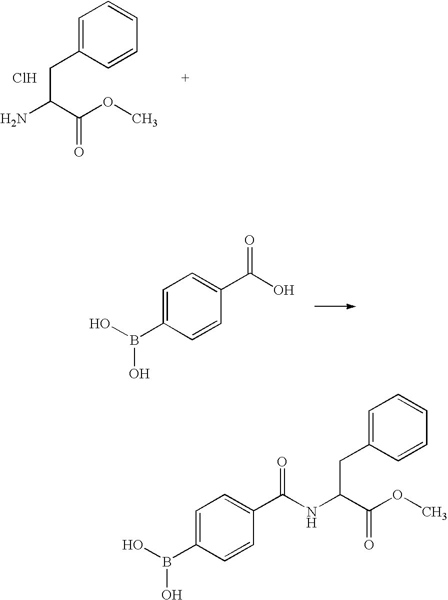

To a mixture of phenylalanine methyl ester hydrochloride (1.090 g, 5.053 mmol), 4-carboxyphenylbronic acid (0.840 g, 5.05 mmol), 1-ethyl-3-(3-dimethylaminopropyl)carbodiimide hydrochloride (0.970 g, 5.05 mmol), 1-hydroxybenzotriazole (0.680 g, 5.05 mmol) and dichloro-methane (20.0 ml) was added triethylamine (0.560 g, 5.53 mmol), and the mixture was stirred at room temperature overnight. The mixture was concentrated under reduced pressure, and the reside was partitionated between ethyl acetate and water. The separated organic phase was washed with saturated aqueous ammonium chloride solution and brine, dried over sodium sulfate, filtered and concentrated under reduced pressure. The crude product was purified by recrystalization from ethyl acetate: hexane twice to give 4[N-(1-methoxycarbonyl-2-phenylethyl)carbamoyl]phenyl-boronic acid (0.710 g, 43%) as a white solid.

### 2-[4-(2-Benzyloxy-pyrimidin-5-yl)-benzoylamino]-3-phenyl-propionic acid methyl ester

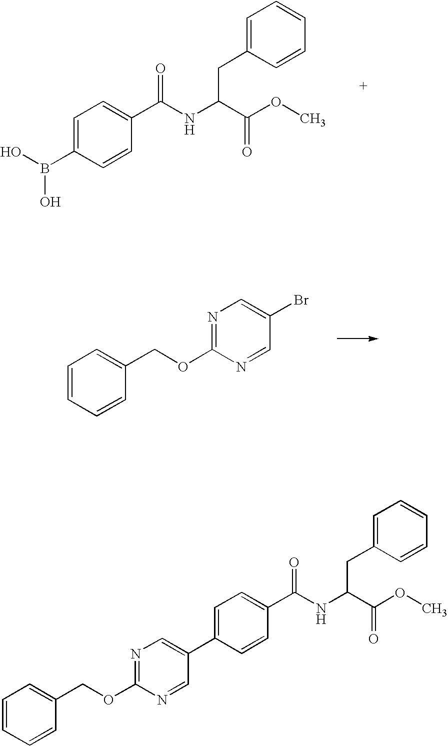

To a mixture of 4-[N-(1-methoxycarbonyl-2-phenylethyl)carbamoyl]phenylboronic acid (0.100 g, 0.306 mmol), 2-benzyloxy-5-bromopyrimidine (0.081 g, 0.31 mmol) and 1,2-dimethoxyethane (2 mL) was added 1.8 N sodium carbonate aqueous solution (0.500 mL, 0.917 mmol) followed by tetrakis(triphenylphosphine)palladium (0.036 g, 0.031 mmol). The mixture was stirred at 90° C. for 1 hour. After cooled to room temperature, the mixture was partitioned between ethyl acetate and water. The separated organic phase was washed with brine, dried over sodium sulfate, filtered and concentrated under reduced pressure. The crude product was suspended in methanol, collected by filtration, washed with methanol, and dried under reduced pressure to give 2-[6(2-benzyloxypyrimidin-5-yl)benzoylamino]-3-phenylpropionic acid methyl ester (0.039 g, 27%) as a white solid.

### 2-[4-(2-Benzyloxypyrimidin-5-yl)benzoylamino]-3-phenylpropionic acid

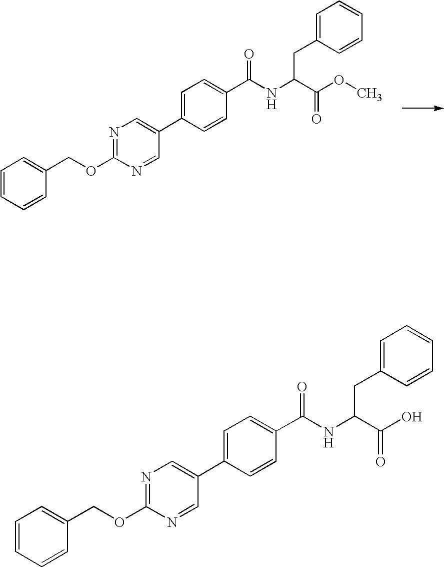

To a solution of 2-[4-(2-benzyloxypyrimidin-5-yl)benzoylamino]-3-phenylpropionic acid methyl ester (0.039 g, 0.082 mmol) in ethanol (0.500 mL), water (1.00 mL) and tetrahydrofuran (1.00 mL) was added 1N aqueous lithium hydroxide solution (0.250 mL, 0.250 mmol), and the mixture was stirred at room temperature for 2 hours. The mixture was partitionated between ethyl acetate and 1 N aqueous HCl solution. The separated organic phase was washed with brine, dried over sodium sulfate, filtered and concentrated under reduced pressure. The residue was purified by re-crystalization from ethyl acetate and hexane to give 2-[4-(2-benzyloxypyrimidin-5-yl)benzoyl-amino]-3-phenylpropionic acid (0.012g, 33%) as a white solid.

Melting point: 184° C.

Molecular weight: 453.5

Mass spectrometry: 454 (M+H)⁺

In vitro activity grade: A

¹H-NMR (500 MHz, DMSO-d6): δ 3.06-3.11 (1H, m), 3.21 (1H, dd, J=4.2, 13.6 Hz), 4.62-4.67 (1H, m), 5.47 (2H, s), 7.17-7.20 (1H, m), 7.27 (2H, t, J=7.3 Hz), 7.32-7.36 (3H, m), 7.41 (2H, t, J=7.3 Hz), 7.48 (2H, d, J=7.6 Hz), 7.84 (2H, d, J=8.2 Hz), 7.91 (2H, d, J=7.9 Hz), 8.76 (1H, d, J=8.2 Hz), 9.02 (2H, s)

### Example 6-1

### 2-Benzyloxy-5-bromopyridine

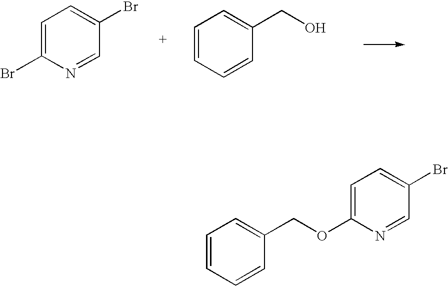

A mixture of 2,5-dibromopyridine (20.0 g, 84.4 mmol), dibenzo-18-crown-6 (1.5 g, 4.2 mmol), benzyl alcohol (11.9 g, 11.4 mL, 109.8 mmol) and KOH (11.4 g, 202.6 mmol) in toluene (200 mL) was refluxed with Dean-Stark for 1.5 hours. After removal of solvent under reduced pressure, the residue was diluted with water, and extracted with chloroform. The separated organic phase was dried over magnesium sulfate, filtered and concentrated under reduced pressure. The crude oil was purified by column chromatography on silica-gel, (hexane:ethyl acetate, 98:2) followed by recrystallization from hexane, to give 2-benzyloxy-5-bromopyridine (20.6 g, 92%) as a colorless solid.

### 2-Benzyloxy-5-tributylstannanylpyridine

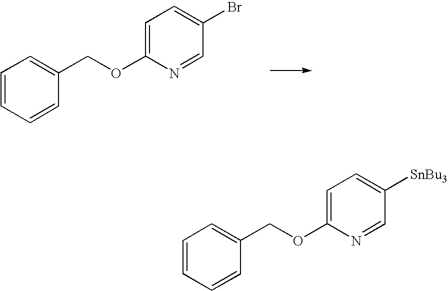

To a cold (−78° C.) solution of 2-benzyloxy-5-bromopyridine (10.0 g, 37.9 mmol) in ether (200 mL) was added n-butyllithium (1.56 M in n-hexane, 29.1 mL, 45.4 mmol). After 30 min, Bu3SnCl was added and the reaction mixture was stirred at −78° C. for further 1 hour and quenched with aqueous potassium fluoride solution. The solution was extracted with ether. The separated organic phase was washed with brine, dried over sodium sulfate, filtered and concentrated under reduced pressure. The residue was purified by column chromatography on silica-gel (hexane:ethyl acetate, 98:2) to give 2-benzyloxy-5-tributylstannanylpyridin (15.4 g, 86%) as a colorless oil.

### 4-(6-Benzyloxypyridin-3-yl)benzoic acid methyl ester

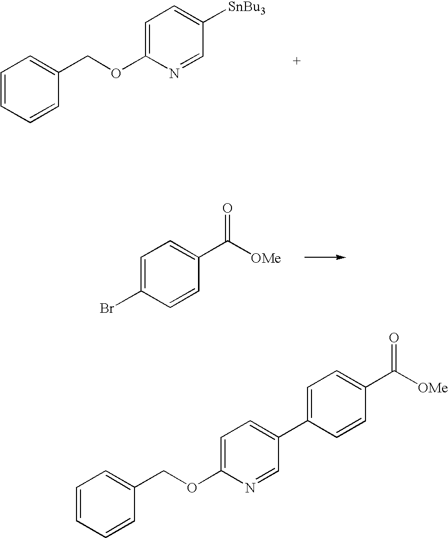

A mixture of methyl 4-bromobenzoate (0.5 g, 2.3 mmol), 2-benzyloxy-5-tributylstannanylpyridine (1.0 g, 2.1 mmol) and tetrakis(triphenylphosphine)palladium (0.15 g, 0.13 mmol) in DMF (10 mL) was heated at 100° C. overnight. After cooled to room temperature, the reaction mixture was quenched with aqueous potassium fluoride solution and stirred for 3 hours at room temperature. The resulting precipitates were removed by filtration and the filtrate was extracted with ethyl acetate. The separated organic layer was washed with brine, dried over sodium sulfate, filtered and concentrated under reduced pressure. The crude product was recrystallized from hexane to give 4-(6-benzyloxypyridin-3-yl)benzoic acid methyl ester (76 mg, 11%) as a colorless solid.

### 4-(6-Benzyloxypyridin-3-yl)benzoic acid

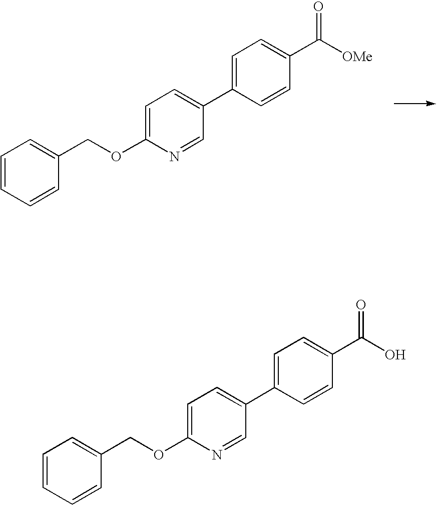

To a solution of 4-(6-benzyloxypyridin-3-yl)benzoic acid methyl ester (0.075 g, 0.23 mmol) in methanol (2 mL) was added 1M NaOH aqueous solution (0.5 mL) at room temperature and the reaction mixture was heated at 90° C. for 1 hour. The solution was diluted with water, acidified by 1N HCl solution, and extracted with ethyl acetate. The separated organic phase was washed with brine, dried over magnesium sulfate, filtered and concentrated under reduced pressure. The crude product was recrystallized from iso-propanol to give 4-(6-benzyloxypyridin-3-yl)benzoic acid (0.048 g, 68%) as a colorless solid.

### 2-[4-(6-Benzyloxypyridin-3-yl)benzoylamino]-3-phenylpropionic acid methyl ester

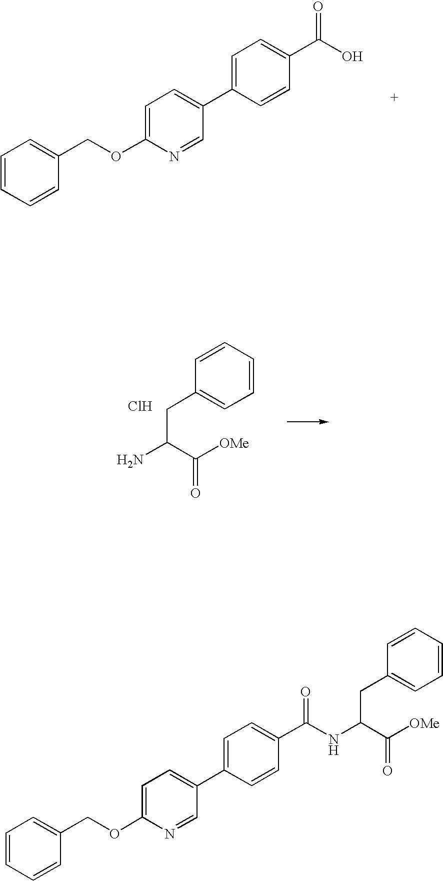

To a solution of 4-(6-benzyloxypyridin-3-yl)benzoic acid (0.045 g, 0.15 mmol), phenylalanine methyl ester hydrochloride (0.038 g, 0.18 mmol) and triethylamine (0.051 mL, 0.37 mmol) in DMF (2 mL) was added benzotriazole-1-yl-oxy-tris(pyrrolidine)phosphonium hexafluorophosphate (0.092 g, 0.18 mmol), and the stirring was continued overnight. The mixture was diluted with water and extracted with ethyl acetate. The separated organic layer was washed with saturated sodium carbonate solution and brine, dried over sodium sulfate, filtered and concentrated under reduced pressure. The residue was purified by column chromatography on silica-gel (hexane: ethyl acetate, 80:20) to give 2-[4-(6-benzyloxypyridin-3-yl)benzoylamino]-3-phenyl-propionic acid methyl ester (0.064 g, 93%) as a colorless oil.

Sodium 2-[4-(6-benzyloxypyridin-3-yl)benzoylamino]-3-phenylpropionate

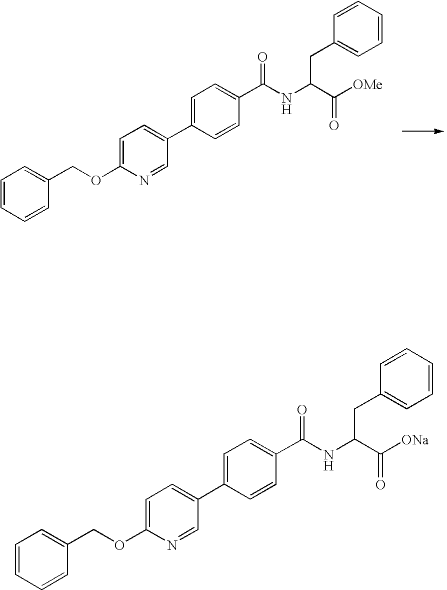

A mixture of 2-[4-(6-benzyloxypyridin-3-yl)benzoylamino]-3-phenylpropionic acid methyl ester (0.062 g, 0.13 mmol) and 1M NaOH aqueous solution (2 mL) in methanol (5 mL) was stirred at room temperature overnight. The resulting precipitates were collected by filtration, washed with ether, and recrystallized from methanol to give sodium 2-[4-(6-benzyloxypyridin-3-yl)benzoylamino]-3-phenylpropionate (0.053 g, 84%) as a colorless solid.

Melting point: 240-242° C.

Molecular weight: 474.49

Mass spectrometry: 453 (M−Na⁺+H)⁺

In vitro activity grade: A

¹H-NMR (500 MHz, DMSOd6): δ 3.08 (1H, dd, J=5.4, 13.0 Hz), 3.21 (1H, dd, J=5.0, 13.0 Hz), 4.10 (1H, q, J=5.4 Hz), 5.41 (2H, s), 6.99 (1H, d, J=8.2 Hz), 7.08 (1H, m), 7.14 (4H, m), 7.33 (1H, t, J=7.3 Hz), 7.39 (2H, t, J=7.3 Hz), 7.47 (2H, d, J=7.3 Hz), 7.74 (4H, s), 7.79 (1H, d, J=6.0 Hz), 8.08 (1H, dd, J=2.5, 8.5 Hz), 8.54 (1H, d, J=2.5 Hz)

### Example 7-1

### 2-(4-Iodobenzoylamino)-3-phenylpropionic acid methyl ester

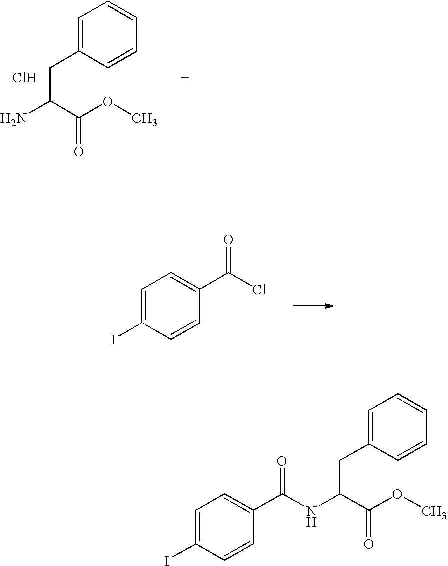

To a mixture of phenylalanine methyl ester hydrochloride (1.000 g, 4.636 mmol), 4-iodobenzyl-chloride (1.300 g, 4.879 mmol) and DMF (25.0 mL) was added N,N-diisopropylethylamnine (0.900 g, 6.96 mmol), and the mixture was stirred at room temperature overnight. The mixture was diluted with ethyl acetate. The organic phase was washed with brine, dried over sodium sulfate, filtered and concentrated under reduced pressure. The crude product was purified by recrystallization from ethyl acetate and hexane twice to give 2-(4-iodobenzoylamino)-3-phenylpropionic acid methyl ester (1.190 g, 63%) as a white solid.

### 2-[(4′-Benzyloxy-3′-fluorobiphenyl-4-carbonyl)amino]-3-phenylpropionic acid methyl ester

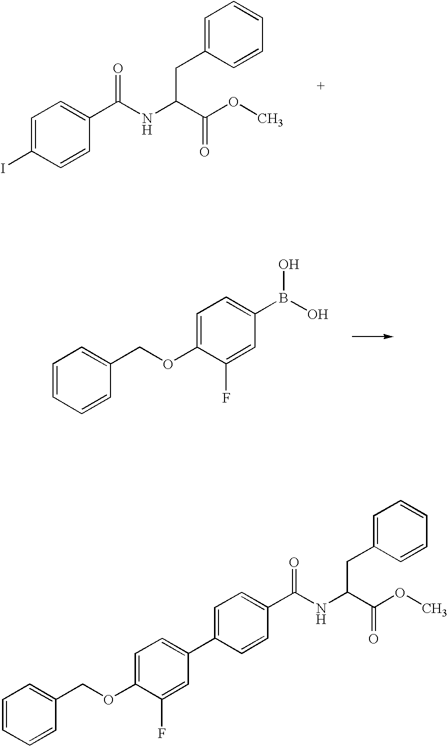

To a mixture of 2-(4-iodobenzoylamino)-3-phenylpropionic acid methyl ester (0.070 g, 0.17 mmol), 4-(benzyloxy)-3-fluorophenylboronic acid (0.042 g, 0.17 mmol) and 1,2-dimethoxyethane (1.0 mL) was added 2.1 N sodium carbonate aqueous solution (0.250 mL, 0.513 mmol) followed by tetrakis(triphenylphosphine)palladium (0.020 g, 0.017 mmol). The mixture was stirred at 90° C. overnight. After cooled to room temperature, the mixture was partitioned between ethyl acetate and water. The separated organic phase was washed with brine, dried over sodium sulfate, filtered and concentrated under reduced pressure. The crude product was purified by preparative TLC (chloroform: ethanol, 60:1) to give 2-[(4′-benzyloxy-3′-fluorobiphenyl-4-carbonyl)amino]-3-phenylpropionic acid methyl ester (0.045 g, 54%) as a white solid.

### 2-[(4′-Benzyloxy-3′-fluorobiphenyl-4-carbonyl)amino]-3-phenylpropionic acid

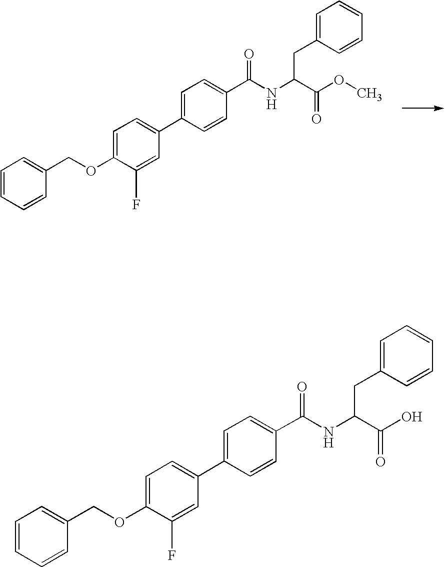

To a solution of 2-[(4′-benzyloxy-3′-fluorobiphenyl-4-carbonyl)amino]-3-phenylpropionic acid methyl ester (0.045 g, 0.092 mmol) in ethanol (0.50 mL), water (1.0 mL) and tetrahydrofuran (1.0 mL) was added 1N lithium hydroxide aqueous solution (0.280 mL, 0.280 mmol), and the mixture was stirred at room temperature for 3 hours. The mixture was acidified by 1 N HCl aqueous solution. The precipitate was collected by filtration and washed with water. The solid was purified by recrystallization from ethyl acetate and hexane to give 2-[(4′-benzyloxy-3′-fluorobiphenyl-4-carbonyl)amino]-3-phenylpropionic acid (0.025 mg, 57%) as a white solid.

Melting point: 215° C.

Molecular weight: 469.51

Mass spectrometry: 470 (M+H)⁺

In vitro activity grade: A

¹H-NMR (500 MHz, DMSO-d6): δ 3.09 (1H, dd, J=11.0, 13.7 Hz), 3.19 (1H, dd, J=4.4, 13.9 Hz), 4.60-4.65 (1H, m), 5.25 (2H, s), 7.17-7.19 (1H, m), 7.25-7.28 (2H, m), 7.32-7.37 (4H, m), 7.41-7.44 (2H, m), 7.48 (2H, d, J=7.3 Hz), 7.52-7.54 (1H, m), 7.67 (1H, dd, J=2.2, 12.6 Hz), 7.76 (2H, d, J=8.5 Hz), 7.86 (2H, d, J=8.2 Hz), 8.75 (1H, d, J=8.2 Hz), 12.80 (1H, s)

### Example 8-1

### 2-[(4′-Hydroxybiphenyl-4-carbonyl)amino]-3-phenylpropionic acid methyl ester

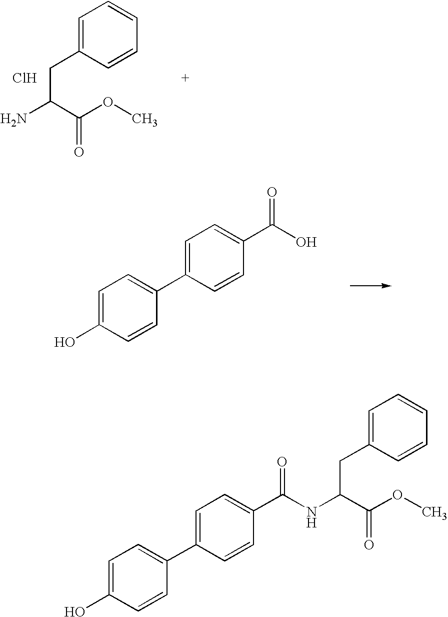

To a mixture of phenylalanine methyl ester hydrochloride (1.980 g, 9.180 mmol), 4′-hydroxy-biphenyl-4-carboxylic acid (2.560 g, 11.95 mmol), 1-ethyl-3-(3-dimethylaminopropyl)carbodiimide hydrochloride (2.280 g, 11.93 mmol), 1-hydroxybenzotriazole (1.610 g, 11.91 mmol) and dichloromethane (50.0 mL) was added triethylamine (1.210 g, 11.96 mmol), and the mixture was stirred at room temperature overnight. The mixture was concentrated under reduced pressure, and the residue was partitionated between ethyl acetate and water. The separated organic phase was washed with aqueous saturated ammonium chloride solution and brine, dried over sodium sulfate, filtered and concentrated under reduced pressure. The crude product was purified by recrystallization from ethyl acetate and hexane twice to give 2-[(4′-hydroxybiphenyl-4 carbonyl)amino]-3-phenylpropionic acid methyl ester (3.090 g, 90%) as a white solid.

### 2-{[4′-(3-Methylbenzyloxy)biphenyl-4-carbonyl]amino}-3-phenylpropionic acid

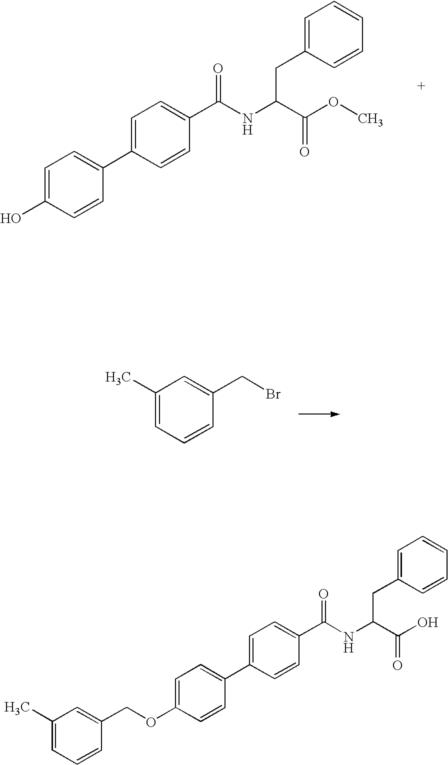

To a mixture of 2-[(4′-hydroxybiphenyl-4carbonyl)amino]-3-phenylpropionic acid methyl ester (0.030 g, 0.080 mmol), 3-methylbenzyl bromide (0.015 g, 0.080 mmol) and DMF (1.2 mL) was added 2-tert-butylimino-2-diethylamino-1,3-dimethyl-perhydro-1,3,2-iazaphosphorine on polystyrene (0.110 g, 0.240 mmol), and the mixture was stirred at 80° C. overnight; After cooled to room temperature, the mixture was concentrated under reduced pressure. The residue was purified by preparative TLC (chloroform: ethanol, 40:1) to give 2-{[4′-(3-methylbenzyloxy)biphenyl-4-carbonyl]amino}-3-phenylpropionic acid methyl ester as a white solid.

The solid was dissolved in ethanol (0.250 mL), water (0.500 mL) and tetrahydroflran (0.500 mL), 1N lithium hydroxide aqueous solution (0.160 mL, 0.160 mmol) was added to the mixture, which was then stirred at room temperature for 3 hours. The mixture was acidified by 1 N HCl aqueous solution. The resultant precipitate was collected by filtration, washed with water, and dried under reduced pressure to give 2-{[4′-(3-methylbenzyloxy)biphenyl-4-carbonyl]amino}-3-phenyl-propionic acid (0.005g, 13%) as a white solid.

Melting point: 204° C.

Molecular weight: 465.5

Mass spectrometry: 466 (M+H)⁺

In vitro activity grade: A

¹H-NMR (500 MHz, DMSO-d6): δ 2.32 (3H, s), 3.09 (1H, dd, J=10.7, 13.9 Hz), 3.20 (1H, dd, J=4.4, 13.9 Hz), 4.61-4.66 (1H, m), 5.12 (2H, s), 7.10-7.12 (2H, m), 7.13-7.18 (2H, m), 7.25-7.29 (5H, m), 7.32 (2H, d, J=7.3 Hz), 7.66-7.68 (2H, m), 7.70 (2H, d, J=8.5 Hz), 7.86 (2H, d, J=8.5 Hz), 8.29 (1H, d, J=8.2 Hz), 12.74 (1H, s)

| Ex | | Mol | | | In |
|---|---|---|---|---|---|
| No | Structure | weight | MASS | MP | vitro |
| 8-2 | 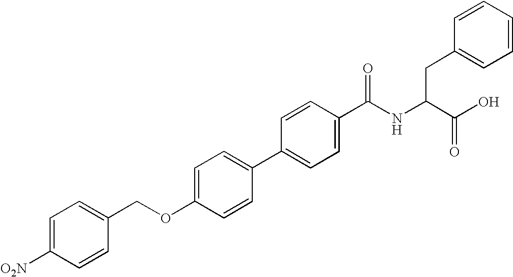 | 496.52 | 497 | 210 | B |
| 8-3 | 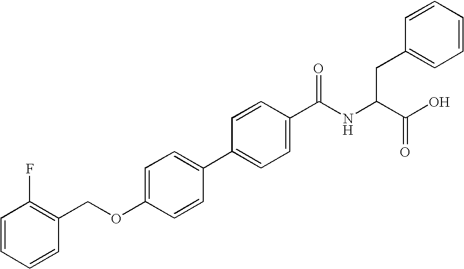 | 469.52 | 470 | 226 | A |
| 8-4 | 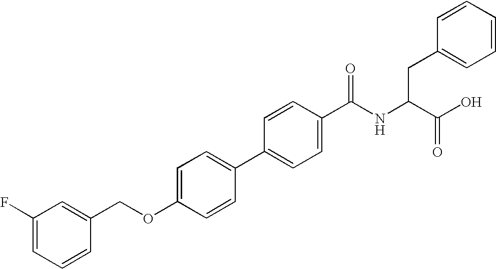 | 469.52 | 470 | 224 | A |
| 8-5 | 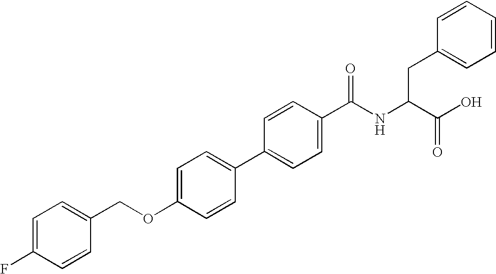 | 469.52 | 470 | 229 | A |
| 8-6 | 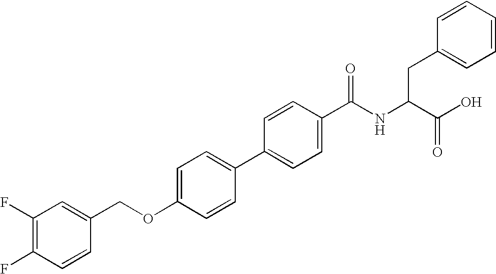 | 487.51 | 488 | 206 | A |
| 8-7 | 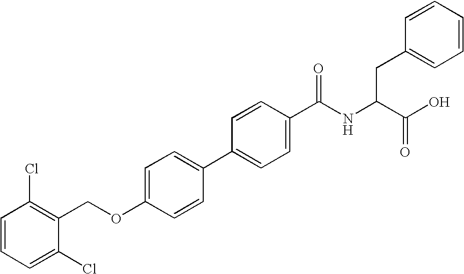 | 520.42 | 520 | 190 | A |
| 8-8 | 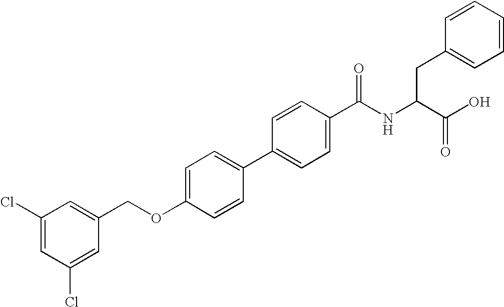 | 520.42 | 520 | 166 | C |
| 8-9 | 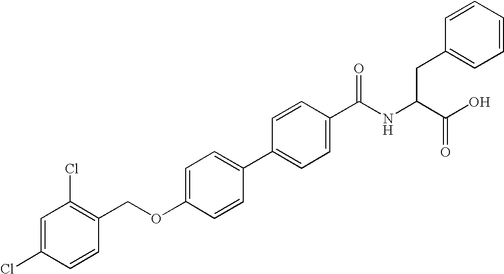 | 520.42 | 520 | 186 | C |
| 8-10 | 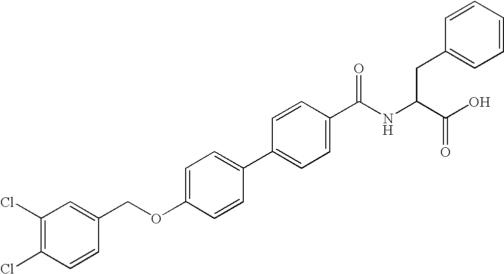 | 520.42 | 520 | 207 | B |
| 8-11 | 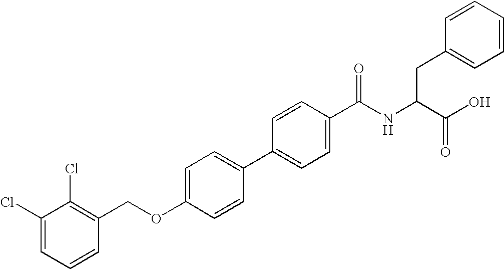 | 520.42 | 520 | 169 | A |
| 8-12 | 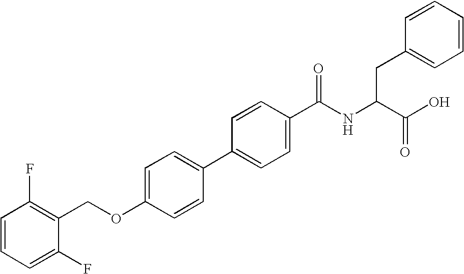 | 487.51 | 488 | 210 | B |
| 8-13 | 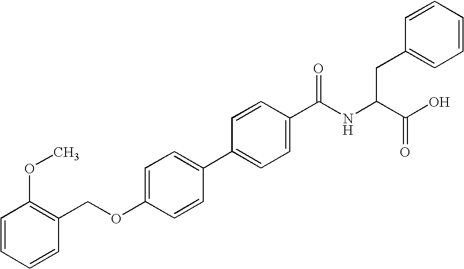 | 481.55 | 482 | 152 | B |
| 8-14 | 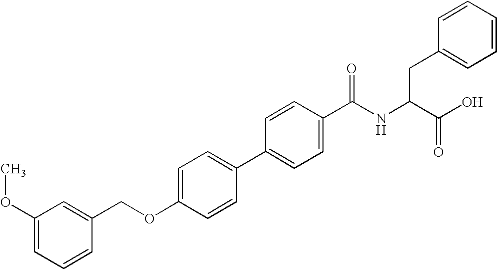 | 481.55 | 482 | 142 | A |
| 8-15 | 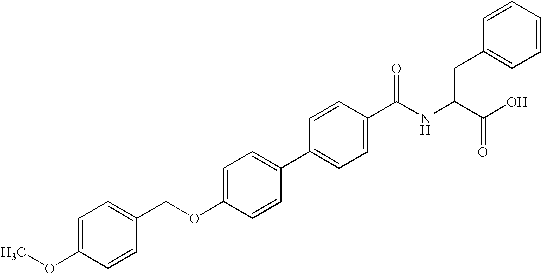 | 481.55 | 482 | 228 | B |
| 8-16 | 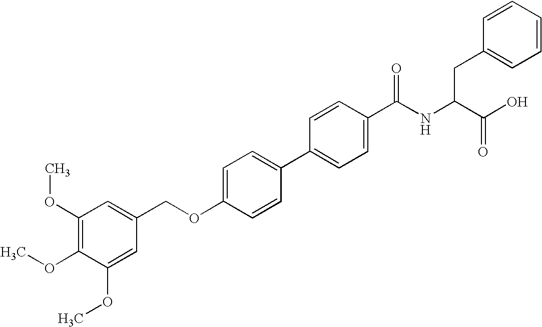 | 541.61 | 542 | 140 | C |
| 8-17 | 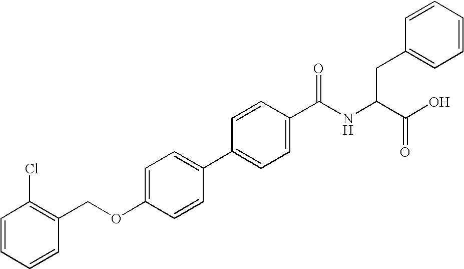 | 485.97 | 486 | 171 | A |
| 8-18 | 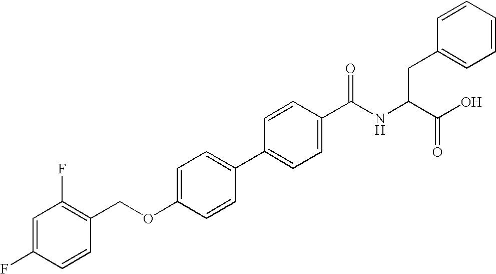 | 487.51 | 488 | 189 | A |
| 8-19 | 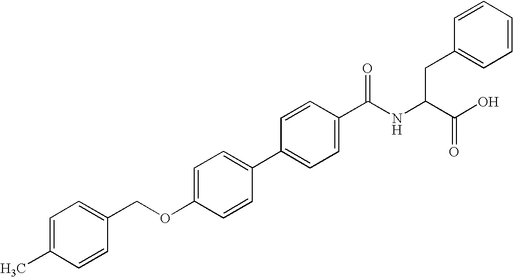 | 465.55 | 466 | 237 | B |
| 8-20 | 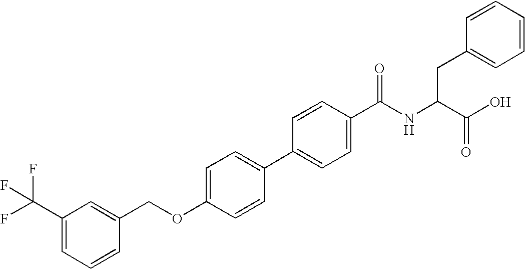 | 519.53 | 520 | 184 | B |
| 8-21 | 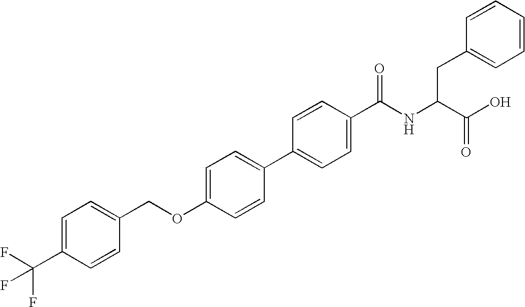 | 519.53 | 520 | 230 | C |
| 8-22 | 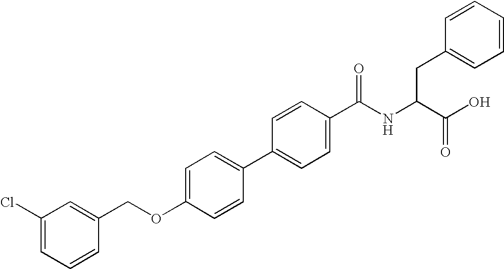 | 485.97 | 484 | 206 | A |
| 8-23 | 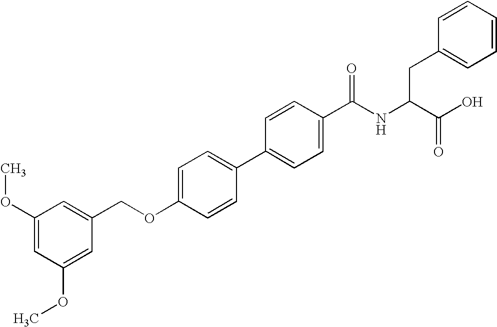 | 511.58 | 512 | 155 | A |
| 8-24 | 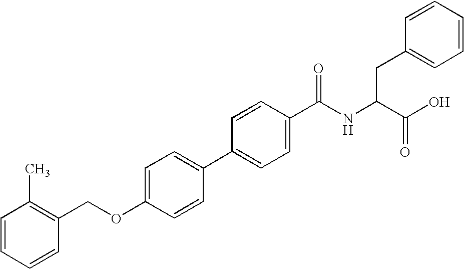 | 465.55 | 466 | 206 | A |
| 8-25 | 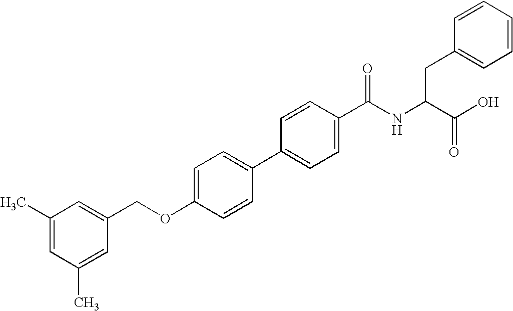 | 479.58 | 480 | 180 | B |
| 8-26 | 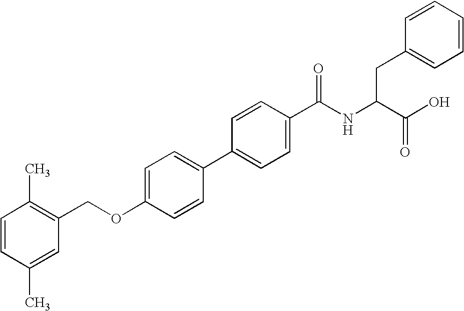 | 479.58 | 480 | 194 | B |
| 8-27 | 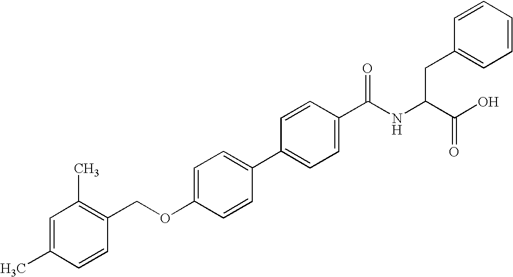 | 479.58 | 480 | 178 | B |
| 8-28 | 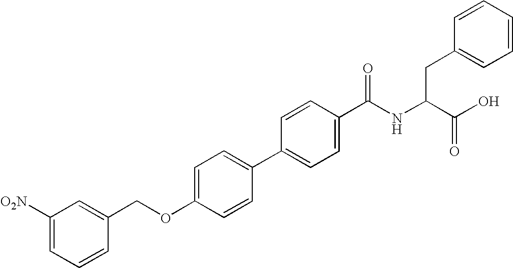 | 496.52 | 495 | 189 | A |
| 8-29 | 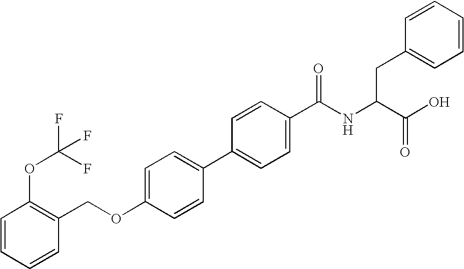 | 535.52 | 534 | 185 | C |
| 8-30 | 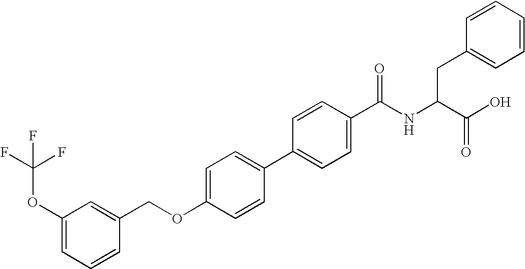 | 535.52 | 536 | 162 | C |
| 8-31 | 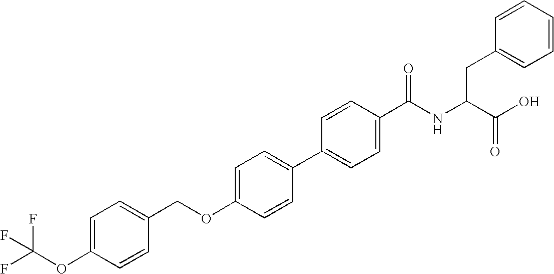 | 535.52 | 534 | 208 | C |
| 8-32 | 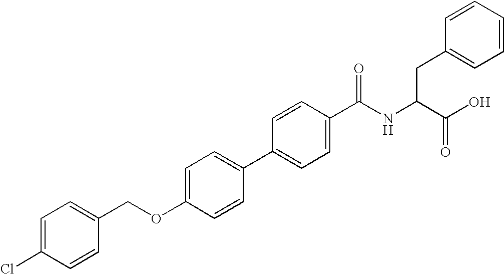 | 485.97 | 485 | 233 | B |
| 8-33 | 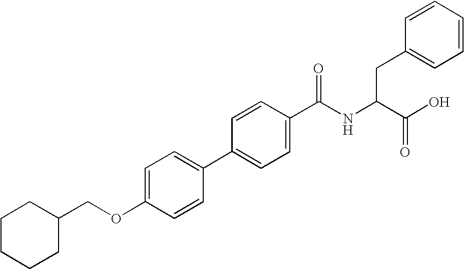 | 457.57 | 458 | 199 | B |
| 8-34 | 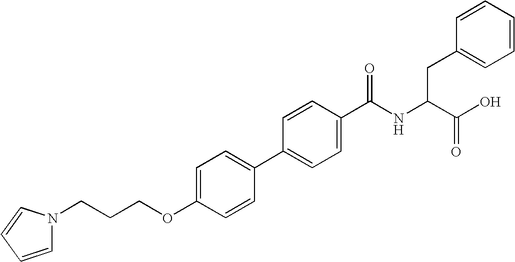 | 468.56 | 467 | 218 | B |
| 8-35 | 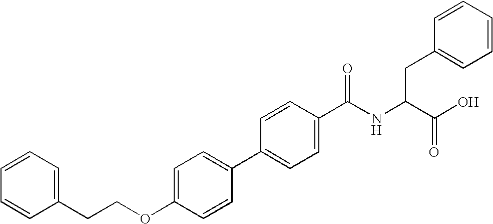 | 465.55 | 466 | 187 | A |
| 8-36 | 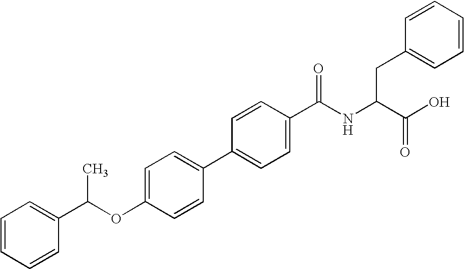 | 465.55 | 466 | 189 | B |
| 8-37 | 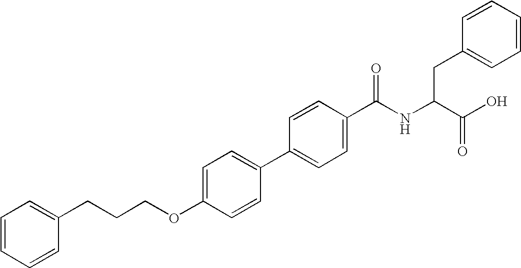 | 479.58 | 480 | 208 | B |
| 8-38 | 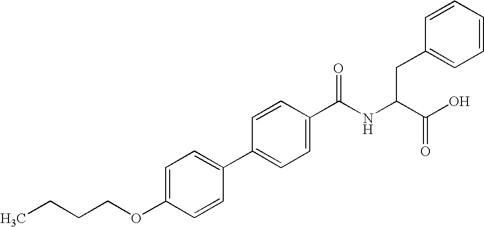 | 417.51 | 418 | 197 | B |
| 8-39 | 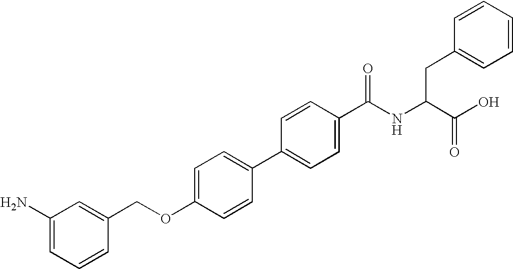 | 466.54 | 467 | 228 | A |
| 8-40 | 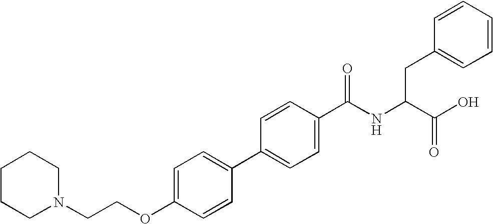 | 472.59 | 473 | 155 | C |

According to the similar synthetic procedure of Example 8-1, compounds shown in Table 8 were prepared.

1. An aryl or heteroaryl amido alkane derivative of the formula (I), its tautomeric or stereoisomeric form, or a salt thereof: 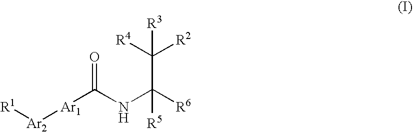
wherein
Ar₁ and Ar₂ independently represent phenyl, or a 5 or 6 membered heteroaromatic ring containing 1 to 4 heteroatoms selected from the group consisting of O, N, and S,
wherein
said phenyl and 5 or 6 membered heteroaromatic ring optionally bear 1 to 3 substituents selected from the group consisting of halogen and (C₁₋₆)alkyl optionally substituted by hydroxy, or mono-, di- or tri-halogen;
R¹ represents —O¹¹, —SR¹¹, —SOR¹¹, —SO₂R¹¹, —NR¹²R¹³, —CHR¹⁴R¹⁵, halogen, hydroxy, cyano, nitro, amino, N—(C₁₋₆)alkylamino, N,N-di(C₁₋₆)alkylamino, aryl, heteroaryl,
(C₁₋₆)alkyl substituted by hydroxy, or mono-, di- or tri-halogen,
(C₁₋₆)alkoxy substituted by hydroxy, or mono-, di- or tri-halogen,
aryl substituted (C₁₋₆)alkoxy(C₁₋₆)alkylene, or
heteroaryl substituted (C₁₋₆) alkoxy(C₁₋₆)alkylene,
wherein
R¹¹ represents
(C₁₋₆)alkyl optionally substituted by a 3 to 10 membered saturated or unsaturated ring having 0 to 3 heteroatoms selected from the group consisting of S, O and N,
(C₂₋₆)alkenyl optionally substituted by aryl or heteroaryl, or
(C₂₋₆)alkynyl optionally substituted by aryl or heteroaryl,
wherein
said 3 to 10 membered saturated or unsaturated ring having 0 to 3 heteroatoms, aryl and heteroaryl optionally bear 1 to 3 substituents selected from the group consisting of halogen, hydroxy, cyano, nitro, amino, N—(C₁₋₆)alkylamino, N,N-di(C₁₋₆)alkylamino, aryl, heteroaryl, (C₁₋₆)alkyl optionally substituted by hydroxyl or mono-, di- or tri-halogen, and (C₁₋₆)alkoxy optionally substituted by mono-, di- or tri-halogen;
R¹² and R¹³ independently represent hydrogen, (C₁₋₆)alkyl optionally substituted by aryl or heteroaryl,
or
R¹² and R¹³ together form with the nitrogen atom, a 5 to 7 membered saturated heterocyclic ring optionally interrupted by O or NH;
R¹⁴ and R¹⁵ independently represent hydrogen, aryl, aryloxy, heteroaryloxy,
(C₁₋₆)alkyl optionally substituted by aryl, heteroaryl, aryloxy, or hetero-aryloxy,
(C₂₋₆)alkenyl optionally substituted by aryl or heteroaryl, or
(C₂₋₆)alkynyl optionally substituted by aryl or heteroaryl,
or
R¹⁴ and R¹⁵ together form, with the CH, a 3-8 membered saturated ring optionally interrupted by NH, or O, or phenyl optionally substituted by hydroxy, halogen or (C₁₋₆)alkyl;
R² represents hydrogen, hydroxy, halogen, cyano, (C₁₋₆)alkoxy, (C₂₋₆)alkenyl, (C₂₋₆)alkynyl, (C₃₋₇)cycloalkyl, amino, N—(C₁₋₆)alkylamino, N,N-di(C₁₋₆)alkylamino, aryl, (C₁₋₆)alkyl optionally substituted by mono-, di- or tri-halogen, or a 5 or 6 membered heteroaromatic ring containing 1 to 4 heteroatoms selected from the group consisting of O, N, and S,
wherein
said aryl and 5 or 6 membered heteroaromatic ring optionally bear 1 to 3 substituents selected from the group consisting of halogen, hydroxy, (C₁₋₆)alkyl, (C₁₋₆)alkoxy, amino, N—(C₁₋₆)alkylamino, N,N-di(C₁₋₆)alkylamino, phenyl and a 5 or 6 membered heteroaromatic ring containing 1 to 4 heteroatoms selected from the group consisting of O, N, and S,
wherein
said phenyl and 5 or 6 membered heteroaromatic ring optionally bear 1 to 3 substituents selected from the group consisting of halogen, hydroxy, (C₁₋₆)alkyl, (C₁₋₆)alkoxy, amino, N—(C₁₋₆)alkylamino, and N,N-di(C₁₋₆)alkylamino;
R³ represents hydrogen, hydroxy, halogen, cyano, (C₁₋₆)alkoxy, (C₂₋₆)alkenyl, (C₂₋₆)alkynyl, (C₃₋₇)cycloalkyl, amino, N—(C₁₋₆)alkylamino, N,N-di(C₁₋₆)alkylamino, phenyl, a 5 or 6 membered heteroaromatic ring containing 1 to 4 heteroatoms selected from the group of O, N, and S, or (C₁₋₆)alkyl optionally substituted by hydroxy or mono-, di- or tri-halogen,
wherein
said phenyl and 5 or 6 membered heteroaromatic ring optionally bear 1 to 3 substituents selected from the group consisting of halogen, hydroxy, (C₁₋₆)alkyl, (C₁₋₆)alkoxy, amino, N—(C₁₋₆)alkylamino, N,N-di(C₁₋₆)alkylamino, phenyl and a 5 or 6 membered heteroaromatic ring containing 1 to 4 heteroatoms selected from the group consisting of O, N, and S,
wherein
said phenyl and 5 or 6 membered heteroaromatic ring optionally bear 1 to 3 substituents selected from the group consisting of halogen, hydroxy, (C₁₋₆)alkyl, (C₁₋₆)alkoxy, amino, N—(C₁₋₆)alkylamino, and N,N-di(C₁₋₆)alkylamino;
R⁴ represents hydrogen, hydroxy, halogen, cyano, (C₁₋₆)alkoxy, (C₂₋₆)alkenyl, (C₂₋₆)alkynyl, (C₃₋₇)cycloalkyl, amino, N—(C₁₋₆)alkylamino, N,N-di(C₁₋₆)alkylamino, phenyl, a 5 or 6 membered heteroaromatic ring containing 1 to 4 heteroatoms selected from the group consisting of O, N, and S, or (C₁₋₆)alkyl optionally substituted by hydroxy or mono-, di- or tri-halogen,
wherein
said phenyl and 5 or 6 membered heteroaromatic ring optionally bear 1 to 3 substituents selected from the group consisting of halogen, hydroxy, (C₁₋₆)alkyl, (C₁₋₆)alkoxy, amino, N—(C₁₋₆)alkylamino, N,N-di(C₁₋₆)alkylamino, phenyl and a 5 or 6 membered heteroaromatic ring containing 1 to 4 heteroatoms selected from the group consisting of O, N, and S,
wherein
said phenyl and 5 or 6 membered heteroaromatic ring optionally bear 1 to 3 substituents selected from the group consisting of halogen, hydroxy, (C₁₋₆)alkyl, (C₁₋₆)alkoxy, amino, N—(C₁₋₆)alkylamino, and N,N-di(C₁₋₆) alkylamino;
R⁵ represents hydrogen, halogen, cyano, or (C₁₋₆)alkyl optionally substituted by hydroxy or mono-, di- or tri-halogen; and
R⁶ represents carboxy or tetrazolyl.

2. The aryl or heteroaryl amido alkane derivative of the formula (I), its tautomeric or stereoisomeric form, or a salt thereof as claimed in claim 1,
wherein
Ar₁ and Ar₂ independently represent phenyl, pyridyl, pyrimidinyl, thienyl, oxazolyl, isoxazolyl, pyrrolyl, imidazolyl, or pyrazolyl
R¹ represents —OR¹¹, —SR¹¹, —SOR¹¹, —SO₂R¹¹, —NR¹²R¹³, —CHR¹⁴R¹⁵, halogen, hydroxy, cyano, nitro, (C₁₋₆)alkoxy, amino, N—(C₁₋₆) alkylamino, N,N-di(C₁₋₆)alkylamino, aryl, heteroaryl, (C₁₋₆)alkyl optionally substituted by hydroxy or mono-, di- or tri-halogen, aryl substituted (C₁₋₆)alkoxy(C₁₋₆)alkylene, or heteroaryl substituted (C₁₋₆)alkoxy(C₁₋₆)alkylene;
wherein
R¹¹ represents (C₁₋₆)alkyl optionally substituted by a 3 to 10 membered saturated or unsaturated ring having 0 to 3 heteroatoms selected from the group consisting of S, O and N, (C₂₋₆)alkenyl optionally substituted by aryl or heteroaryl, or (C₂₋₆)alkynyl optionally substituted by aryl or heteroaryl,
wherein
said a 3 to 10 membered saturated or unsaturated ring having 0 to 3 heteroatoms, aryl and heteroaryl optionally bear 1 to 3 substituents selected from the group consisting of halogen, hydroxy, cyano, nitro, amino, N—(C₁₋₆)alkylamino, N,N-di(C₁₋₆)alkylamino, aryl, heteroaryl, (C₁₋₆)alkyl optionally substituted by hydroxy, or mono-, di- or tri-halogen, and (C₁₋₆)alkoxy optionally substituted by mono-, di- or tri- halogen;
R¹² and R¹³ independently represent hydrogen, or (C₁₋₆)alkyl optionally substituted by aryl or heteroaryl,
or
R¹² and R¹³ together form, with the nitrogen atom, a 5 to 7 membered saturated heterocyclic ring optionally interrupted by O or NH,
R¹⁴ and R¹⁵ independently represent hydrogen, aryloxy, heteroaryloxy,
(C₁₋₆)alkyl optionally substituted by aryl, heteroaryl, aryloxy, or heteroaryloxy,
(C₂₋₆)alkenyl optionally substituted by aryl or heteroaryl, or
(C₂₋₆)alkynyl optionally substituted by aryl or heteroaryl,
or
R¹⁴ and R¹⁵ together form, with the CH, a 3 to 8 membered saturated ring optionally interrupted by NH, or O, or phenyl optionally substituted by hydroxy, halogen or (C₁₋₆)alkyl;
R² represents hydrogen, hydroxy, halogen, cyano, (C₁₋₆)alkoxy, (C₂₋₆)alkenyl, (C₂₋₆)alkynyl, (C₃₋₇)cycloalkyl, amino, N—(C₁₋₆)alkylamino, N,N-di(C₁₋₆)alkylamino, aryl, (C₁₋₆)alkyl optionally substituted by hydroxy or mono-, di- or tri-halogen, or a 5 or 6 membered heteroaromatic ring containing 1 to 4 heteroatoms selected from the group consisting of O, N, and S,
wherein
said aryl and 5 or 6 membered heteroaromatic ring optionally bear 1 to 3 substituents selected from the group consisting of halogen, hydroxy, (C₁₋₆)alkyl, (C₁₋₆)alkoxy, amino, N—(C₁₋₆)alkylamino, N,N-di(C₁₋₆)alkylamino, phenyl and a 5 or 6 membered heteroaromatic ring containing 1 to 4 heteroatoms selected from the group consisting of O, N, and S,
wherein
said phenyl and 5 or 6 membered heteroaromatic ring optionally bear 1 to 3 substituents selected from the group consisting of halogen, hydroxy, (C₁₋₆)alkyl, (C₁₋₆ )alkoxy, amino, N—(C₁₋₆)alkylamino, and N,N-di(C₁₋₆)alkylamino;
R³ represents hydrogen, hydroxy, halogen, cyano, (C₁₋₆)alkoxy, (C₂₋₆)alkenyl, (C₂₋₆)alkynyl, (C₃₋₇)cycloalkyl, amino, N—(C₁₋₆)alkylamino, N,N-di(C₁₋₆)alkylamino, phenyl, a 5 or 6 membered heteroaromatic ring containing 1 to 4 heteroatoms selected from the group consisting of O, N, and S, or (C₁₋₆)alkyl optionally substituted by hydroxy or mono-, di- or tri-halogen,
wherein
phenyl and 5 or 6 membered heteroaromatic ring optionally bear 1 to 3 substituents selected from the group consisting of halogen, hydroxy, (C₁₋₆)alkyl, (C₁₋₆)alkoxy, amino, N—(C₁₋₆)alkylamino, N,N-di(C₁₋₆)alkylamino, phenyl and a 5 or 6 membered heteroaromatic ring containing 1 to 4 heteroatoms selected from the group consisting of O, N, and S,
wherein
said phenyl and 5 or 6 membered heteroaromatic ring optionally bear 1 to 3 substituents selected from the group consisting of halogen, hydroxy, (C₁₋₆)alkyl, (C₁₋₆)alkoxy, amino, N—(C₁₋₆)alkylamino, and N,N-di(C₁₋₆) alkylamino;
R⁴ represents hydrogen, hydroxy, halogen, cyano, (C₁₋₆)alkoxy, (C₂₋₆)alkenyl, (C₂₋₆)alkynyl, (C₃₋₇)cycloalkyl, amino, N—(C₁₋₆)alkylamino, N,N-di(C₁₋₆)alkylamino, phenyl, a 5 or 6 membered heteroaromatic ring containing 1 to 4 heteroatoms selected from the group consisting of O, N, and S, or (C₁₋₆)alkyl optionally substituted by hydroxy or mono-, di- or tri-halogen,
wherein
said phenyl and 5 or 6 membered heteroaromatic ring optionally bear 1 to 3 substituents selected from the group consisting of halogen, hydroxy, (C₁₋₆)alkyl, (C₁₋₆)alkoxy, amino, N—(C₁₋₆)alkylamino, N,N-di(C₁₋₆)alkylamino, phenyl and a 5 or 6 membered heteroaromatic ring containing 1 to 4 heteroatoms selected from the group consisting of O, N, and S,
wherein
said phenyl and 5 or 6 membered heteroaromatic ring optionally bear 1 to 3 substituents selected from the group consisting of halogen, hydroxy, (C₁₋₆)alkyl, (C₁₋₆)alkoxy, amino, N—(C₁₋₆)alkylamino, and N,N-di(C₁₋₆) alkylamino;
R⁵ represents hydrogen, halogen, cyano or (C₁₋₆)alkyl optionally substituted by mono-, di- or tri-halogen; and
R⁶ represents carboxy or tetrazolyl.

3. The aryl or heteroaryl amido alkane derivative of claim 1, represented by the formula (I-i), its tautomeric or stereoisomeric form, or a salt thereof: 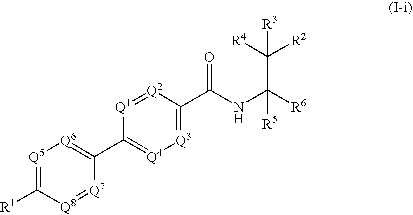
wherein
Q ¹, Q², Q³, Q⁴, Q⁵, Q⁶, Q⁷and Q⁸ independently represent CH, CR⁷or N;
wherein
R⁷ represents halogen or (C₁₋₆)alkyl optionally substituted by mono-, di- or tri-halogen;
R¹ represents —OR¹¹, —SR¹¹, —SOR¹¹, —SO₂R¹¹, —NR¹²R¹³, —CHR¹⁴R¹⁵,
aryl substituted (C₁₋₆)alkoxy(C₁₋₆)alkylene, or
heteroaryl substituted (C₁₋₆)alkoxy(C₁₋₆)alkylene,
wherein
R¹¹ represents (C₁₋₆)alkyl optionally substituted by a 5 to 10 membered saturated or unsaturated ring having 0 to 3 heteroatoms selected from the group consisting of S, O and N, (C₂₋₆)alkenyl optionally substituted by aryl or heteroaryl, or (C₂₋₆)alkynyl optionally substituted by aryl or heteroaryl,
wherein
said 5 to 10 membered saturated or unsaturated ring, aryl and heteroaryl optionally bear 1 to 3 substituents selected from the group consisting of halogen, hydroxy, cyano, nitro, amino, N—(C₁₋₆)-alkylamino, N,N-di(C₁₋₆)alkylamino, aryl, heteroaryl, (C₁₋₆)alkyl optionally substituted by mono-, di- or tri-halogen, and (C₁₋₆)alkoxy optionally substituted by mono-, di- or tri-halogen;
R¹² and R¹³ independently represent hydrogen, (C₁₋₆)alkyl optionally substituted by aryl or heteroaryl, (C₂₋₆)alkenyl optionally substituted by aryl or heteroaryl, or (C₂₋₆)alkynyl optionally substituted by aryl or heteroaryl,
or
R¹² and R¹³ together form with the nitrogen atom, a 5 to 7 membered saturated heterocyclic ring optionally interrupted by O or NH;
R¹⁴ and R¹⁵ independently represent hydrogen, aryloxy, heteroaryloxy, (C₁₋₆)alkyl optionally substituted by aryl, heteroaryl, aryloxy, or heteroaryloxy,
(C₂₋₆)alkenyl optionally substituted by aryl or heteroaryl, (C₂₋₆)alkynyl optionally substituted by aryl or heteroaryl,
or
R¹⁴ and R¹⁵ together form with the CH, a 5 to 7 membered saturated ring optionally interrupted by NH, O or phenyl optionally substituted by hydroxy, halogen or (C₁₋₆)alkyl;
R² represents hydrogen, hydroxy, halogen, cyano, (C₁₋₆)alkoxy, (C₂₋₆)alkenyl, (C₂₋₆)alkynyl, (C₃₋₇)cycloalkyl, amino, N—(C₁₋₆)alkylamino, N,N-di(C₁₋₆)alkylamino, phenyl, (C₁₋₆)alkyl optionally substituted by mono-, di- or tri-halogen, or a 5 or 6 membered heteroaromatic ring containing 1 to 4 heteroatoms selected from the group consisting of O, N, and S,
wherein
said phenyl and 5 or 6 membered heteroaromatic ring optionally bear 1 to 3 substituents selected from the group consisting of halogen, hydroxy, (C₁₋₆)alkyl, (C₁₋₆)alkoxy, amino, N—(C₁₋₆)alkylamino, N,N-di(C₁₋₆)alkylamino, phenyl and a 5 or 6 membered heteroaromatic ring containing 1 to 4 heteroatoms selected from the group consisting of O, N, and S,
wherein
said phenyl and heteroaromatic ring optionally bear 1 to 3 substituents selected from the group consisting of halogen, hydroxy, (C₁₋₆)alkyl, (C₁₋₆)alkoxy, amino, N—(C₁₋₆)alkylamino, and N,N-di(C₁₋₆)alkylamino;
R³ represents hydrogen, hydroxy, halogen, cyano, (C₁₋₆)alkoxy, (C₂₋₆)alkenyl, (C₂₋₆)alkynyl, (C₃₋₇)cycloalkyl, amino, N—(C₁₋₆)alkylamino, N,N-di(C₁₋₆)alkylamino, phenyl, a 5 or 6 membered heteroaromatic ring containing 1 to 4 heteroatoms selected from the group consisting of O, N, and S, or (C₁₋₆)alkyl optionally substituted by mono-, di- or tri-halogen,
wherein
said phenyl and 5 or 6 membered heteroaromatic ring are optionally bear 1 to 3 substituents selected from the group consisting of halogen, hydroxy, (C₁₋₆)alkyl, (C₁₋₆)alkoxy, amino, N—(C₁₋₆)alkylamino, N,N-di(C₁₋₆)alkylamino, phenyl and a 5 or 6 membered heteroaromatic ring containing 1 to 4 heteroatoms selected from the group consisting of O, N, and S,
wherein
said phenyl and 5 or 6 membered heteroaromatic ring optionally bear 1 to 3 substituents selected from the group consisting of halogen, hydroxy, (C₁₋₆)alkyl, (C₁₋₆)alkoxy, amino, N—(C₁₋₆)alkylamino, and N,N-di(C₁₋₆)alkylamino;
R⁴ represents hydrogen, hydroxy, halogen, cyano, (C₁₋₆)alkoxy, (C₂₋₆)alkenyl, (C₂₋₆)alkynyl, (C₃₋₇)cycloalkyl, amino, N—(C₁₋₆)alkylamino, N,N-di(C₁₋₆)alkylamino, phenyl, (C₁₋₆)alkyl optionally substituted by mono-, di- or tri-halogen, or a 5 or 6 membered heteroaromatic ring containing 1 to 4 heteroatoms selected from the group consisting of O, N, and S,
wherein
said phenyl and 5 or 6 membered heteroaromatic ring optionally bear 1 to 3 substituents selected from the group consisting of halogen, hydroxy, (C₁₋₆)alkyl, (C₁₋₆)alkoxy, amino, N—(C₁₋₆)alkylamino, N,N-di(C₁₋₆)alkylamino, phenyl and a 5 or 6 membered heteroaromatic ring containing 1 to 4 heteroatoms selected from the group consisting of O, N, and S,
wherein
said phenyl and 5 or 6 membered heteroaromatic ring optionally bear 1 to 3 substituents selected from the group consisting of halogen, hydroxy, (C₁₋₆)alkyl, (C₁₋₆)alkoxy, amino, N—(C₁₋₆)alkylamino, and N,N-di(C₁₋₆)alkylamino;
R⁵ represents hydrogen, halogen, cyano, or (C₁₋₆)alkyl optionally substituted by mono-, di- or tri-halogen; and
R⁶ represents carboxy or tetrazolyl.

4. The aryl or heteroaryl amido alkane derivative of the formula (I-i), its tautomeric or stereoisomeric form, or a salt thereof as claimed in claim 3,
wherein
Q¹ represents N;
Q², Q³, Q⁴, Q⁵, Q⁶, Q⁷ and Q⁸ independently represent CH or CR⁷;
wherein
R⁷ represents halogen or (C₁₋₆)alkyl optionally substituted by mono-, di- or tri-halogen;
R represents —OR¹¹,
wherein
R¹¹ represents (C₁₋₆)alkyl optionally substituted by a 5 to 7 membered saturated or unsaturated ring having 0 to 3 heteroatoms selected from the group consisting of S, O and N,
wherein
said 5 to 7 membered saturated or unsaturated ring optionally bears 1 to 3 substituents selected from the group consisting of halogen, hydroxy, cyano, nitro, amino, N—(C₁₋₆)alkylamino, N,N-di(C₁₋₆)alkylamino, aryl, heteroaryl, (C₁₋₆)alkyl optionally substituted by -mono-, di- or tri- halogen, and (C₁₋₆)alkoxy optionally substituted by mono-, di- or tri-halogen;
R³ represents hydrogen;
R⁴ represents hydrogen;
R⁵ represents hydrogen; and
R⁶ represents carboxy.

5. The aryl or heteroaryl amido alkane derivative of the formula (I-i), its tautomeric or stereoisomeric form, or a salt thereof as claimed in claim 3,
wherein
Q² represents N;
Q¹, Q³, Q⁴, Q⁵, Q⁶, Q⁷ and Q⁸ independently represent CH or CR⁷;
wherein
R⁷ represents halogen or (C₁₋₆)alkyl optionally substituted by mono-, di- or tri-halogen;
R¹ represents —OR¹¹,
wherein
R¹¹ represents (C₁₋₆)alkyl optionally substituted by a 5 to 10 membered saturated or unsaturated ring having 0 to 3 heteroatoms selected from the group consisting of S, O and N,
wherein
said 5 to 10 membered saturated or unsaturated ring optionally bears 1 to 3 substituents selected from the group consisting of halogen, hydroxy, cyano, nitro, amino, N—(C₁₋₆)alkylamino, N,N-di(C₁₋₆)alkylamino, aryl, heteroaryl, (C₁₋₆)alkyl optionally substituted by mono-, di- or tri-halogen and (C₁₋₆)alkoxy optionally substituted by mono-, di- or tri-halogen;
R³ represents hydrogen;
R⁴ represents hydrogen;
R⁵ represents hydrogen; and
R⁶ represents carboxy.

6. The aryl or heteroaryl amido alkane derivative of the formula (I-i), its tautomeric or stereoisomeric form, or a salt thereof as claimed in claim 3,
wherein
Q⁵ and Q⁸ independently represent CH or N;
Q¹, Q², Q³, Q⁴, Q⁶ and Q⁷ independently represent CH or CR⁷;
wherein
R⁷ represents halogen or (C₁₋₆)alkyl optionally substituted by mono-, di- or tri-halogen;
R¹ represents —OR¹¹,
wherein
R¹¹ represents (C₁₋₆)alkyl optionally substituted by a 5 to 10 membered saturated or unsaturated ring having 0 to 3 heteroatoms selected from the group consisting of S, O and N,
wherein
said 5 to 10 membered saturated or unsaturated ring optionally bears 1 to 3 substituents selected from the group consisting of halogen, hydroxy, cyano, nitro, amino, N—(C₁₋₆)alkylamino, N,N-di(C₁₋₆)alkylamino, aryl, heteroaryl, (C₁₋₆)alkyl optionally substituted by mono-, di- or tri-halogen and (C₁₋₆)alkoxy optionally substituted by mono-, di- or tri-halogen;
R³ represents hydrogen;
R⁴ represents hydrogen;
R⁵ represents hydrogen; and
R⁶ represents carboxy.

7. The aryl or heteroaryl amino alkane derivative of the formula (I-i), its tautomeric or stereoisomeric form, or a salt thereof as claimed in claim 3,
wherein
Q¹ represents N;
Q², Q³, Q⁴, Q⁵, Q⁶, Q⁷ and Q⁸ independently represent CH;
R¹ represents aryl(C₁₋₆)alkoxy, aryl(C₁₋₆)alkoxy(C₁₋₆)alkylene or aryloxy (C₁₋₆)alkylene,
wherein
said aryl and aryl moieties of said aryloxy optionally bear 1 to 3 substituents selected from the group consisting of halogen, hydroxy, cyano, nitro, amino, N—(C₁₋₆)alkylamino, N,N-di(C₁₋₆)alkylamino, (C₁₋₆)alkyl optionally substituted by mono-, di- or tri-halogen, and (C₁₋₆)alkoxy optionally substituted by mono-, di- or tri-halogen;
R² represents halogen, hydroxy, (C₃₋₇)cycloalkyl, or phenyl optionally bearing 1 to 3 substituents selected from the group consisting of halogen, hydroxy, amino, N—(C₁₋₆)alkylamino, N,N-di(C₁₋₆)alkylamino, and phenyl optionally substituted by halogen, hydroxy or amino;
R³ represents hydrogen;
R⁴ represents hydrogen;
R⁵ represents hydrogen; and
R⁶ represents carboxy.

8. The aryl or heteroaryl amido alkane derivative of the formula (I-i), its tautomeric or stereoisomeric form, or a salt as claimed in claim 3,
wherein
Q² represent N;
Q¹, Q³, Q⁴,Q⁵, Q⁶, Q⁷ and Q⁸ independently represent CH;
R¹ represents aryl(C₁₋₆)alkoxy, aryl(C₁₋₆)alkoxy(C₁₋₆)alkylene or aryloxy(C₁₋₆)alkyl;
wherein
said aryl and aryl moiety of said aryloxy are optionally having optionally bear 1 to 3 substituents selected from the group consisting of halogen, hydroxy, cyano, nitro, amino, N—(C₁₋₆)alkylamino, N,N-di(C₁₋₆)alkylamino, (C₁₋₆)alkyl optionally substituted by mono-, di- or tri-halogen, and (C₁₋₆)alkoxy optionally substituted by mono-, di- or tri-halogen;
R² represents halogen, hydroxy, (C₃₋₇)cycloalkyl, or phenyl optionally bearing 1 to 3 substituents selected from the group consisting of halogen, hydroxy, amino, N—(C₁₋₆)alkylamino, N,N-di(C₁₋₆)alkylamino and phenyl optionally substituted by halogen, hydroxy or amino;
R³ represents hydrogen;
R⁴ represents hydrogen;
R⁵ represents hydrogen; and
R⁶ represents carboxy.

9. The aryl or heteroaryl amido alkane derivative of the formula (I-i), its tautomeric or stereoisomeric form, or a salt thereof as claimed in claim 3,
wherein
Q⁵ and Q⁸ independently represent CH or N;
Q¹, Q², Q³, Q⁴, Q⁶ and Q⁷ independently represent CH;
R¹ represents aryl(C₁₋₆)alkoxy, aryl(C₁₋₆)alkoxy(C₁₋₆)alkylene or aryloxy(C₁₋₆)alkyl;
wherein
said aryl and aryl moiety of said aryloxy optionally bear 1 to 3 substituents selected from the group consisting of halogen, hydroxy, cyano, nitro, amino, N—(C₁₋₆)alkylamino, N,N-di(C₁₋₆)alkylamino, (C₁₋₆)alkyl optionally substituted by mono-, di- or tri-halogen, and (C₁₋₆)alkoxy optionally substituted by mono-, di- or tri-halogen;
R² represents halogen, hydroxy, (C₃₋₇)cycloalkyl, or phenyl optionally bearing 1 to 3 substituents selected from the group consisting of halogen, hydroxy, amino, N—(C₁₋₆)alkylamino, N,N-di(C₁₋₆)alkylamino and phenyl optionally substituted by halogen, hydroxy or amino;
R³ represents hydrogen;
R⁴ represents hydrogen;
R⁵ represents hydrogen; and
R⁶ represents carboxy.

10. The aryl or heteroaryl amido alkane derivative of the formula (I-i), its tautomeric or stereoisomeric form, or a salt thereof as claimed in claim 3,
wherein
Q¹, Q², Q³, Q⁴, Q⁵, Q⁶, Q⁷ and Q⁸ independently represent CH or CR⁷;
wherein
R⁷ represents halogen or (C₁₋₆) alkyl optionally substituted by mono-, di- or tri-halogen;
R¹ represents aryl(C₁₋₆)alkoxy, heteroaryl(C₁₋₆)alkoxy, (C₅₋₇)cycloalkyl(C₁₋₆)alkoxy, aryl(C₁₋₆)alkoxy(C₁₋₆)alkylene or aryloxy(C₁₋₆)alkylene;
wherein
said aryl, heteroaryl, (C₅₋₇)cycloalkyl, and aryl moiety of said aryloxy optionally bear 1 to 3 substituents selected from the group consisting of halogen, hydroxy, cyano, nitro, amino, N—(C₁₋₆)alkylamino, N,N-di(C₁₋₆)alkylamino, (C₁₋₆)alkyl optionally substituted by mono-, di- or tri-halogen, and (C₁₋₆)alkoxy optionally substituted by mono-, di- or tri-halogen;
R² represents halogen, hydroxy, (C₅₋₇)cycloalkyl, or phenyl optionally having bearing 1 to 3 substituents selected from the group consisting of halogen, hydroxy, amino, N—(C₁₋₆)alkylamino, N,N-di(C₁₋₆)alkylamino, and phenyl optionally substituted by halogen, hydroxy or amino;
R³ represents hydrogen;
R⁴ represents hydrogen;
R⁵ represents hydrogen; and
R⁶ represents carboxy.

11. The aryl or heteroaryl amino alkane derivative of the formula (I-i), its tautomeric or stereoisomeric form, or a salt thereof as claimed in claim 3,
wherein
Q¹ represent N;
Q², Q³, Q⁴, Q⁵, Q⁶, Q⁷ and Q⁸ independently represent CH;
R¹ represents phenyl(C₁₋₆)alkoxy, phenyl(C₁₋₆)alkoxy(C₁₋₆)alkylene or phenoxy(C₁₋₆)alkylene,
wherein
said phenyl and phenyl moiety of said phenoxy optionally bear 1 to 3 substituents selected from the group consisting of halogen, hydroxy, cyano, nitro, amino, N—(C₁₋₆)alkylamino, N,N-di(C₁₋₆)alkylamino, (C₁₋₆)alkyl optionally substituted by mono-, di- or tri-halogen and (C₁₋₆)alkoxy optionally substituted by mono-, di- or tri-halogen;
R² represents halogen, hydroxy, (C₅₋₇)cycloalkyl, or phenyl optionally bearing 1 to 3 substituents selected from the group consisting of halogen, hydroxy, amino, N—(C₁₋₆)alkylamino, N,N-di(C₁₋₆)alkylamino, and phenyl optionally substituted by halogen, hydroxy or amino;
R³ represents hydrogen;
R⁴ represents hydrogen;
R⁵ represents hydrogen; and
R⁶ represents carboxy.

12. The aryl or heteroaryl amido alkane derivative of the formula (I-i), its tautomeric or stereoisomeric form, or a salt as claimed in claim 3,
wherein
Q² represent N;
Q¹, Q³, Q⁴ Q⁵, Q⁶, Q⁷ and Q⁸ independently represent CH;
R¹ represents phenyl(C₁₋₆)alkoxy, phenyl(C₁₋₆)alkoxy(C₁₋₆)alkylene or phenoxy(C₁₋₆)alkylene;
wherein
said phenyl and phenyl moiety of said phenoxy optionally bear 1 to 3 substituents selected from the group consisting of halogen, hydroxy, cyano, nitro, amino, N—(C₁₋₆)alkylamino, N,N-di(C₁₋₆)alkylamino, (C₁₋₆)alkyl optionally substituted by mono-, di- or tri-halogen, and (C₁₋₆)alkoxy optionally substituted by mono-, di- or tri-halogen;
R² represents halogen, hydroxy, (C₅₋₇)cycloalkyl, or phenyl optionally bearing 1 to 3 substituents selected from the group consisting of halogen, hydroxy, amino, N—(C₁₋₆)alkylamino, N,N-di(C₁₋₆)alkylamino, and phenyl optionally substituted by halogen, hydroxy or amino;
R³ represents hydrogen;
R⁴ represents hydrogen;
R⁵ represents hydrogen; and
R⁶ represents carboxy.

13. The aryl or heteroaryl amido alkane derivative of the formula (I-i), its tautomeric or stereoisomeric form, or a salt thereof as claimed in claim 3,
wherein
Q⁵ and Q⁸ independently represent CH or N;
Q¹, Q², Q³, Q⁴, Q⁶ and Q⁷ independently represent CH;
R¹ represents phenyl(C₁₋₆)alkoxy, phenyl(C₁₋₆)alkoxy(C₁₋₆)alkylene or phenoxy (C₁₋₆)alkylene;
wherein
said phenyl and phenyl moiety of said phenoxy optionally bear 1 to 3 substituents selected from the group consisting of halogen, hydroxy, cyano, nitro, amino, N—(C₁₋₆)alkylamino, N,N-di(C₁₋₆)alkylamino, (C₁₋₆)alkyl optionally substituted by mono-, di- or tri-halogen, and (C₁₋₆)alkoxy optionally substituted by mono-, di- or tri-halogen;
R² represents halogen, hydroxy, (C₅₋₇)cycloalkyl, or phenyl optionally bearing 1 to 3 substituents selected from the group consisting of halogen, hydroxy, amino, N—(C₁₋₆)alkylamino, N,N-di(C₁₋₆)alkylamino, and phenyl optionally substituted by halogen, hydroxy or amino;
R³ represents hydrogen;
R⁴ represents hydrogen;
R⁵ represents hydrogen; and
R⁶ represents carboxy.

14. The aryl or heteroaryl amido alkane derivative of the formula (I-i), its tautomeric or stereoisomeric form, or a salt thereof as claimed in claim 3,
wherein
Q¹, Q², Q³, Q⁴, Q⁵, Q⁶, Q⁷ and Q⁸ independently represent CH or CR⁷;
wherein
R⁷ represents halogen or (C₁₋₆)alkyl optionally substituted by mono-, di- or tri-halogen;
R¹ represents phenyl(C₁₋₆)alkoxy, phenyl(C₁₋₆)alkoxy(C₁₋₆)alkylene or phenoxy(C₁₋₆)alkylene;
wherein
said phenyl and phenyl moiety of said phenoxy optionally bear 1 to 3 substituents selected from the group consisting of halogen, hydroxy, cyano, nitro, amino, N-(C₁₋₆)alkylamino, N,N-di(C₁₋₆)alkylamino, (C₁₋₆)alkyl optionally substituted by mono-, di- or tri-halogen and (C₁₋₆)alkoxy optionally substituted by mono-, di- or tri-halogen;
R² represents halogen, hydroxy, (C₅₋₇)cycloalkyl, or phenyl optionally bearing 1 to 3 substituents selected from the group consisting of halogen, hydroxy, amino, N-(C₁₋₆)alkylamino, N,N-di(C₁₋₆)alkylamino, and phenyl optionally substituted by halogen, hydroxy or amino;
R³ represents hydrogen;
R⁴ represents hydrogen;
R⁵ represents hydrogen; and
R⁶ represents carboxy.

15. The aryl or heteroaryl amido alkane derivative of the formula (I-i), its tautomeric or stereoisomeric-form, or a salt thereof as claimed in claim 3,
wherein
Q¹, Q², Q³, Q⁴, Q⁵, Q⁶, Q⁷ and Q⁸ independently represent CH or CR⁷;
wherein
R⁷ represents fluoro, chloro, bromo, or (C₁₋₆)alkyl optionally substituted by mono-, di- or tri-halogen;
R¹ represents (C₁₋₄)alkoxy optionally substituted by phenyl, cyclohexyl, pyrrolyl or piperidino;
wherein
said phenyl optionally bears 1 to 3 substituents selected from the group consisting of fluoro, chloro, bromo, hydroxy, cyano, nitro, amino, methyl, methoxy, trifluoromethyl, and trifluoromethoxy;
R² represents (C₅₋₇)cycloalkyl, or phenyl;
R³ represents hydrogen;
R⁴ represents hydrogen;
R⁵ represents hydrogen; and
R⁶ represents carboxy.

16. The aryl or heteroaryl amido alkane derivative of claim 1, represented by the formula (I-ii), its tautomeric or stereoisomeric form, or a salt thereof: 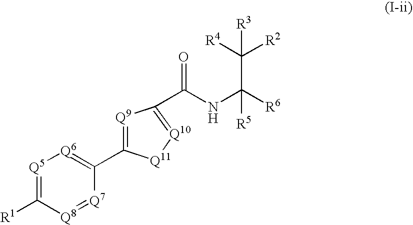
wherein
Q⁵, Q⁶, Q⁷, Q⁸, Q⁹ and Q¹⁰, independently represent CH or N;
Q¹¹ represents CH₂, S, NH, or O;
R¹ represents —OR¹¹, —SR¹¹, —SOR¹¹, —SO₂R¹¹, —NR ¹²R¹³, —CHR¹⁴R¹⁵, aryl substituted (C₁₋₆)alkoxy(C₁₋₆)alkylene, or heteroaryl substituted by(C₁₋₆)alkoxy (C₁₋₆)alkylene,
wherein
R¹¹ represents (C₁₋₆)alkyl optionally substituted by a 3 to 10 membered saturated or unsaturated ring having 0 to 3 heteroatoms selected from the group consisting of S, O and N, (C₂₋₆)alkenyl optionally substituted by aryl or heteroaryl, or (C₂₋₆)alkynyl optionally substituted by aryl or heteroaryl,
wherein
said 3 to 10 membered saturated or unsaturated ring, aryl and heteroaryl optionally bear 1 to 3 substituents selected from the group consisting of halogen, hydroxy, cyano, nitro, amino, N—(C₁₋₆)alkylamino, N,N-di(C₁₋₆)alkylamino, aryl, heteroaryl,
(C₁₋₆)alkyl optionally substituted by mono-, di- or tri-halogen, and
(C₁₋₆)alkoxy optionally substituted by mono-, di- or tri-halogen,
R¹²and R¹³ independently represent hydrogen, (C₁₋₆)alkyl optionally substituted by aryl or heteroaryl, (C₂₋₆)alkenyl optionally substituted by aryl or heteroaryl, or (C₂₋₆)alkynyl optionally substituted by aryl or heteroaryl,
or
R¹² and R¹³ together form with the nitrogen atom, a 5 to 7 membered saturated heterocyclic ring optionally interrupted by O or NH;
R¹⁴ and R¹⁵ independently represent hydrogen, aryloxy, heteroaryloxy, (C₁₋₆) alkyl optionally substituted by aryl, heteroaryl, aryloxy, or heteroaryloxy, (C₂₋₆)alkenyl optionally substituted by aryl or heteroaryl, (C₂₋₆)alkynyl optionally substituted by aryl or heteroaryl,
wherein
said aryl and aryl moiety of said aryloxy optionally bear 1 to 3 substituents selected from the group consisting of halogen, hydroxy, cyano, nitro, amino, N—(C₁₋₆)alkylamino, N,N-di(C₁₋₆)alkylamino, (C₁₋₆)alkyl optionally substituted by mono-, di- or tri-halogen, and (C₁₋₆)alkoxy optionally substituted by mono-, di- or tri-halogen;
or
R¹⁴ and R¹⁵ together form with the CH, a 5 to 7 membered saturated ring optionally interrupted by NH, or O, or phenyl optionally substituted by hydroxy, halogen or (C₁₋₆)alkyl;
R² represents hydrogen, hydroxy, halogen, cyano, (C₁₋₆)alkoxy, (C₂₋₆)alkenyl, (C₂₋₆)alkynyl, (C₃₋₇)cycloalkyl, amino, N—(C₁₋₆)alkylamino, N,N-di(C₁₋₆)alkylamino, aryl, a 5 or 6 membered heteroaromatic ring containing 1 to 4 heteroatoms selected from the group of O, N, and S, or (C₁₋₆) alkyl optionally substituted by mono-, di- or tri-halogen,
wherein
said aryl and a 5 or 6 membered heteroaromatic ring optionally bear 1 to 3 substituents selected from the group consisting of halogen, hydroxy, (C₁₋₆)alkyl, (C₁₋₆)alkoxy, amino, N-(C₁₋₆)alkylamino, N,N-di(C₁₋₆)alkylamino, phenyl and a 5 or 6 membered heteroaromatic ring containing 1 to 4 heteroatoms selected from the group of O, N, and S,
wherein
said phenyl and heteroaromatic ring optionally bear 1 to 3 substituents selected from the group consisting of halogen, hydroxy, (C₁₋₆)alkyl, (C₁₋₆)alkoxy, amino, N—(C₁₋₆)alkylamino, and N,N-di(C₁₋₆)alkylamino;
R³ represents hydrogen;
R⁴ represents hydrogen;
R⁵ represents hydrogen, hydroxy, cyano, or (C₁₋₆)alkyl optionally substituted by mono-, di- or tri-halogen; and
R⁶ represents carboxy or tetrazolyl.

17. The aryl or heteroaryl amido alkane derivative of the formula (I-ii), its tautomeric or stereoisomeric form, or a salt thereof as claimed in claim 16,
wherein
Q⁵, Q⁶, Q⁷, Q⁸ and Q⁹ represent CH;
Q¹⁰ represents CH or N;
Q¹¹ represents CH₂, NH, or O;
R¹ represents phenyl(C₁₋₆)alkoxy, phenyl(C₁₋₆)alkoxy(C₁₋₆)alkylene or phenoxy(C₁₋₆)alkylene,
wherein
said phenyl and phenyl moiety of said phenoxy optionally bear 1 to 3 substituents selected from the group consisting of halogen, hydroxy, cyano, nitro, amino, N—(C₁₋₆)alkylamino, N,N-di(C₁₋₆)alkylamino, (C₁₋₆)alkyl optionally substituted by mono-, di- or tri-halogen, and (C₁₋₆)alkoxy optionally substituted by mono-, di- or tri-halogen;
R² represents halogen, hydroxy, (C₅₋₇)cycloalkyl, or phenyl optionally bearing 1 to 3 substituents selected from the group consisting of by halogen, hydroxy, amino, N—(C₁₋₆)alkylamino, N,N-di(C₁₋₆)alkylamino, and phenyl optionally substituted by halogen, hydroxy or amino;
R³ represents hydrogen;
R⁴ represents hydrogen;
R⁵ represents hydrogen; and
R⁶ represents carboxy.

18. The aryl or heteroaryl amido alkane derivative of claim 1, represented by the formula (I-iii), its tautomeric or stereoisomeric form, or a salt thereof: 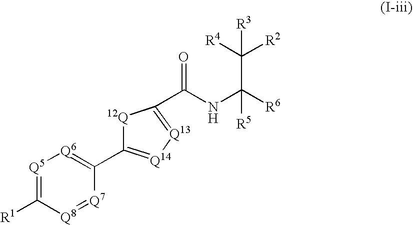
wherein
Q⁵, Q⁶, Q⁷, Q⁸, Q¹³ and Q¹⁴ represent CH;
Q¹² represents CH₂, NH, O, or S;
R¹ represents phenyl(C₁₋₆)alkoxy, phenyl(C₁₋₆)alkoxy(C₁₋₆)alkylene or phenoxy(C₁₋₆)alkylene,
wherein
said phenyl and phenyl moiety of said phenoxy optionally bear 1 to 3 substituents selected from the group consisting of halogen, hydroxy, cyano, nitro, amino, N—(C₁₋₆)alkylamino, N,N-di(C₁₋₆)alkylamino, (C₁₋₆)alkyl optionally substituted by mono-, di- or tri-halogen, and (C₁₋₆)alkoxy optionally substituted by mono-, di- or tri-halogen;
R² represents halogen, hydroxy, (C₅₋₇)cycloalkyl, or phenyl optionally having bearing 1 to 3 substituents selected from the group consisting of halogen, hydroxy, amino, N—(C₁₋₆)alkylamino, N,N-di(C₁₋₆)alkylamino and phenyl optionally substituted by halogen, hydroxy or amino;
R³ represents hydrogen;
R⁴ represents hydrogen;
R⁵ represents hydrogen; and
R⁶ represents carboxy.

19. The aryl or heteroaryl amino alkane derivative, its tautomeric or stereo-isomeric form, or a salt thereof as claimed in claim 1, wherein said derivative is selected from the group consisting of the following compounds:
2-{[5-(4-benzyloxyphenyl)-1H-pyrazole-3-carbonyl]amino}-3-phenylpropionic acid;
2-{[5-(4-benzyloxyphenyl)isoxazole-3-carbonyl]amino}-3-phenylpropionic acid;
2-{[5-(4-benzyloxyphenyl)thiophene-2-carbonyl]amino}-3-phenyl-propionic acid;
2-[(4′-benzyloxybiphenyl-4-carbonyl)amino]-3-phenylpropionic acid;
2-{[6-(4-benzyloxyphenyl)pyridine-3-carbonyl]amino}-3-phenyl-propionic acid;
2-[4-(2-benzyloxypyrimidin-5-yl)benzoylamino]-3-phenylpropionic acid;
2-[4-(6-benzyloxypyridin-3-yl)benzoylamino]-3-phenylpropionic acid;
2-[(4′-benzyloxy-3′-fluorobiphenyl-4-carbonyl)amino]-3-phenylpropionic acid;
2-{[4′-(3-methylbenzyloxy)biphenyl-4-carbonyl]amino}-3-phenylpropionic acid;
2-{[4′-(2-Fluoro-benzyloxy)-biphenyl-4-carbonyl]-amino}-3-phenyl-propionic acid;
2-{[4′-(3-Fluoro-benzyloxy)-biphenyl-4-carbonyl]-amino}-3-phenyl-propionic acid;
2-{[4′-(4-Fluoro-benzyloxy)-biphenyl-4-carbonyl]-amino}-3-phenyl-propionic acid;
2-{[4′-(3,4-Difluoro-benzyloxy)-biphenyl-4-carbonyl]-amino}-3-phenyl-propionic acid;
2-{[4′-(2,6-Dichloro-benzyloxy)-biphenyl-4-carbonyl]-amino}-3-phenyl-propionic acid;
2-{[4′-(2,3-Dichloro-benzyloxy)-biphenyl-4-carbonyl]-amino}-3-phenyl-propionic acid;
2-{[4′-(3-Methoxy-benzyloxy)-biphenyl-4-carbonyl]-amino}-3-phenyl-propionic acid;
2-{[4′-(2-Chloro-benzyloxy)-biphenyl-4-carbonyl]-amino}-3-phenyl-propionic acid;
2-{[4′-(2,4-Difluoro-benzyloxy)-biphenyl-4-carbonyl]-amino} -3-phenyl-propionic acid;
2-{[4′-(3-Chloro-benzyloxy)-biphenyl-4-carbonyl]-amino}-3-phenyl-propionic acid;
2-{[4′-(3,5-Dimethoxy-benzyloxy)-biphenyl-4-carbonyl]-amino}-3-phenyl-propionic acid;
2-{[4′-(2-Methyl-benzyloxy)-biphenyl-4-carbonyl]-amino}-3-phenyl-propionic acid;
2-{[4′-(3-Nitro-benzyloxy)-biphenyl-4-carbonyl]-amino }-3-phenyl-propionic acid;
2-[(4′-Phenethyloxy-biphenyl-4-carbonyl)-amino]-3-phenyl-propionic acid; and
2-{[4′-(3-Amino-benzyloxy)-biphenyl-4-carbonyl]-amino}-3-phenyl-propionic acid.

20. A pharmaceutical composition comprising the compound, its tautomeric or stereoisomeric form, or a physiologically acceptable salt thereof as claimed in claim 1 as an active ingredient, plus a pharmaceutically acceptable carrier.

21. The pharmaceutical composition as claimed in claim 20, further comprising one or more pharmaceutically acceptable excipients.

22. The pharmaceutical composition as claimed in claim 20, wherein the compound, its tautomeric or stereoisomeric form, or a physiologically acceptable salt thereof is a PGI2 antagonist.

23. A method for prophylaxis and/or treatment of a urological disorder or disease comprising administering to a patient an effective amount of a compound according to claim 1 or a tautomeric or stereoisomeric form or physiologically acceptable salt thereof.

24. A method for the prophylaxis and/or treatment of pain comprising administering to a patient an effective amount of a compound according to claim 1 or a tautomeric or stereoisomeric form or physiologically acceptable salt thereof.

25. A method for the prophylaxis and/or treatment of hypotension comprising administering to a patient an effective amount of a compound according to claim 1 or a tautomeric or stereoisomeric form or physiologically acceptable salt thereof.

26. A method for the prophylaxis and/or treatment of hemophilia and hemorrhage comprising administering to a patient an effective amount of a compound according to claim 1 or a tautomeric or stereoisomeric form or physiologically acceptable salt thereof.

27. A method for the prophylaxis and/or treatment of inflammation comprising administering to a patient an effective amount of a compound according to claim 1 or a tautomeric or stereoisomeric form or physiologically acceptable salt thereof.

28. (canceled)

29. A method for controlling urological disorders in humans and animals comprising administration to a human or animal in need thereof a PGI2-antagonisticly effective amount of at least one compound according to claim 1.
